SEQUENCE LISTING (133 sequences)

SEQ ID NO: 1 - protein, 47 aa, synthetic construct
  REGION 1..47: note = Description of sequence: Kind: Leucine Zippers , Name: RR12EE345L zipper
LEIRAAFLRQRNTALRTEVAELEQEVQRLENEVSQYETRYGPLGGGK

SEQ ID NO: 2 - protein, 47 aa, synthetic construct
  REGION 1..47: note = Description of sequence: Kind: Leucine Zippers , Name: 2N two N terminal mutations
LEIEAAFLRQENTALRTEVAELEQEVQRLENEVSQYETRYGPLGGGK

SEQ ID NO: 3 - protein, 47 aa, synthetic construct
  REGION 1..47: note = Description of sequence: Kind: Leucine Zippers , Name: EE12RR345L zipper
LEIEAAFLERENTALETRVAELRQRVQRLRNRVSQYRTRYGPLGGGK

SEQ ID NO: 4 - protein, 47 aa, synthetic construct
  REGION 1..47: note = Description of sequence: Kind: Leucine Zippers , Name: 3N three N terminal mutations
LEIEAAFLRQENTALRTRVAELEQEVQRLENEVSQYETRYGPLGGGK

SEQ ID NO: 5 - protein, 47 aa, synthetic construct
  REGION 1..47: note = Description of sequence: Kind: Leucine Zippers , Name: 3C three C terminal mutations
LEIRAAFLRQRNTALRTRVAELEQRVQRLENRVSQYETRYGPLGGGK

SEQ ID NO: 6 - protein, 47 aa, synthetic construct
  REGION 1..47: note = Description of sequence: Kind: Leucine Zippers , Name: 1N single N terminal mutation
LEIEAAFLRQRNTALRTEVAELEQEVQRLENEVSQYETRYGPLGGGK

SEQ ID NO: 7 - protein, 47 aa, synthetic construct
  REGION 1..47: note = Description of sequence: Kind: Leucine Zippers , Name: 1M single mutation in middle of zipper
LEIRAAFLRQRNTALRTRVAELEQEVQRLENEVSQYETRYGPLGGGK

SEQ ID NO: 8 - protein, 47 aa, synthetic construct
  REGION 1..47: note = Description of sequence: Kind: Leucine Zippers , Name: 2M two mutations in middle of zipper
LEIRAAFLRQRNTALRTRVAELEQRVQRLENEVSQYETRYGPLGGGK

SEQ ID NO: 9 - protein, 38 aa, synthetic construct
  REGION 1..38: note = Description of sequence: Kind: Leucine Zippers , Name: synZip-9 zipper
GSQKVESLKQKIEELKQRKAQLKNDIANLEKEIAYAET

SEQ ID NO: 10 - protein, 44 aa, synthetic construct
  REGION 1..44: note = Description of sequence: Kind: Leucine Zippers , Name: Fos zipper
GSELTDTLQAETDQLEDEKSALQTEIANLLKEKEKLEFILAAHR

SEQ ID NO: 11 - protein, 44 aa, synthetic construct
  REGION 1..44: note = Description of sequence: Kind: Leucine Zippers , Name: Jun zipper
ERISRLEEKVKTLKSQNTELASTASLLREQVAQLKQKVLSHVLE

SEQ ID NO: 12 - protein, 20 aa, synthetic construct
  REGION 1..20: note = Description of sequence: Kind: Signal peptides, Name: mouse Ig kappa signal peptide
METDTLLLWVLLLWVPGSTG

SEQ ID NO: 13 - protein, 27 aa, synthetic construct
  REGION 1..27: note = Description of sequence: Kind: Signal peptides, Name: mouse CD8 signal peptide
MASPLTRFLSLNLLLLGESIILGSGEA

SEQ ID NO: 14 - protein, 20 aa, synthetic construct
  REGION 1..20: note = Description of sequence: Kind: Signal peptides, Name: mouse IL-2 signal peptide
MYSMQLASCVTLTLVLLVNS

SEQ ID NO: 15 - protein, 21 aa, synthetic construct
  REGION 1..21: note = Description of sequence: Kind: Signal peptides, Name: human CD8 signal peptide
MALPVTALLLPLALLLHAARP

SEQ ID NO: 16 - protein, 21 aa, synthetic construct
  REGION 1..21: note = Description of sequence: Kind: Signal peptides, Name: mouse Fas signal peptide
MLWIWAVLPLVLAGSQLRVHT

SEQ ID NO: 17 - protein, 6 aa, synthetic construct
  REGION 1..6: note = Description of sequence: Kind: Linkers, Hinge, spacers, extracellular domains, Name: Linker1
GSGRSG

SEQ ID NO: 18 - protein, 9 aa, synthetic construct
  REGION 1..9: note = Description of sequence: Kind: Linkers, Hinge, spacers, extracellular domains, Name: Linker2
GGGGSGGGS

SEQ ID NO: 19 - protein, 17 aa, synthetic construct
  REGION 1..17: note = Description of sequence: Kind: Linkers, Hinge, spacers, extracellular domains, Name: Linker3
GGGGSGGGGSGGGGSTS

SEQ ID NO: 20 - protein, 12 aa, synthetic construct
  REGION 1..12: note = Description of sequence: Kind: Linkers, Hinge, spacers, extracellular domains, Name: Linker4
GPPVASGSGRSG

SEQ ID NO: 21 - protein, 12 aa, synthetic construct
  REGION 1..12: note = Description of sequence: Kind: Linkers, Hinge, spacers, extracellular domains, Name: Linker5
GGGGSGGGSGSL

SEQ ID NO: 22 - protein, 37 aa, synthetic construct
  REGION 1..37: note = Description of sequence: Kind: Linkers, Hinge, spacers, extracellular domains, Name: Linker6
GGGGSGGGSELPTQGTFSNVSTNVSGGGGSGGGSRSG

SEQ ID NO: 23 - protein, 12 aa, synthetic construct
  REGION 1..12: note = Description of sequence: Kind: Linkers, Hinge, spacers, extracellular domains, Name: Linker7
TSGGGGSGGGSG

SEQ ID NO: 24 - protein, 14 aa, synthetic construct
  REGION 1..14: note = Description of sequence: Kind: Linkers, Hinge, spacers, extracellular domains, Name: Linker8
TSGGGGSGGGSGTG

SEQ ID NO: 25 - protein, 12 aa, synthetic construct
  REGION 1..12: note = Description of sequence: Kind: Linkers, Hinge, spacers, extracellular domains, Name: Linker9
GGGGSGGGSGTG

SEQ ID NO: 26 - protein, 11 aa, synthetic construct
  REGION 1..11: note = Description of sequence: Kind: Linkers, Hinge, spacers, extracellular domains, Name: Linker10
STGGGGSGGGS

SEQ ID NO: 27 - protein, 11 aa, synthetic construct
  REGION 1..11: note = Description of sequence: Kind: Linkers, Hinge, spacers, extracellular domains, Name: Linker11
GSGGGGSGGGS

SEQ ID NO: 28 - protein, 10 aa, synthetic construct
  REGION 1..10: note = Description of sequence: Kind: Linkers, Hinge, spacers, extracellular domains, Name: Linker12
GTSRAKRGSG

SEQ ID NO: 29 - protein, 8 aa, synthetic construct
  REGION 1..8: note = Description of sequence: Kind: Linkers, Hinge, spacers, extracellular domains, Name: Linker13
GGGGSGTG

SEQ ID NO: 30 - protein, 9 aa, synthetic construct
  REGION 1..9: note = Description of sequence: Kind: Linkers, Hinge, spacers, extracellular domains, Name: Linker14
GTSRAKRGS

SEQ ID NO: 31 - protein, 9 aa, synthetic construct
  REGION 1..9: note = Description of sequence: Kind: Linkers, Hinge, spacers, extracellular domains, Name: Linker15
ALGGSGGGS

SEQ ID NO: 32 - protein, 7 aa, synthetic construct
  REGION 1..7: note = Description of sequence: Kind: Linkers, Hinge, spacers, extracellular domains, Name: Linker16
GGGGSTS

SEQ ID NO: 33 - protein, 7 aa, synthetic construct
  REGION 1..7: note = Description of sequence: Kind: Linkers, Hinge, spacers, extracellular domains, Name: Linker17
SGGGGSD

SEQ ID NO: 34 - protein, 6 aa, synthetic construct
  REGION 1..6: note = Description of sequence: Kind: Linkers, Hinge, spacers, extracellular domains, Name: Linker18
GSSGSG

SEQ ID NO: 35 - protein, 6 aa, synthetic construct
  REGION 1..6: note = Description of sequence: Kind: Linkers, Hinge, spacers, extracellular domains, Name: Linker19
GSGGTR

SEQ ID NO: 36 - protein, 5 aa, synthetic construct
  REGION 1..5: note = Description of sequence: Kind: Linkers, Hinge, spacers, extracellular domains, Name: Linker20
GSGTR

SEQ ID NO: 37 - protein, 5 aa, synthetic construct
  REGION 1..5: note = Description of sequence: Kind: Linkers, Hinge, spacers, extracellular domains, Name: Linker21
TSGSG

SEQ ID NO: 38 - protein, 5 aa, synthetic construct
  REGION 1..5: note = Description of sequence: Kind: Linkers, Hinge, spacers, extracellular domains, Name: Linker22
GSGGS

SEQ ID NO: 39 - protein, 18 aa, synthetic construct
  REGION 1..18: note = Description of sequence: Kind: Linkers, Hinge, spacers, extracellular domains, Name: Linker 23 (interchain linker)
GSTGGGGSGGGGSGGGGS

SEQ ID NO: 40 - protein, 19 aa, synthetic construct
  REGION 1..19: note = Description of sequence: Kind: Linkers, Hinge, spacers, extracellular domains, Name: Linker 24
GGGGSGGGGSGGGGSGGGS

SEQ ID NO: 41 - protein, 15 aa, synthetic construct
  REGION 1..15: note = Description of sequence: Kind: Linkers, Hinge, spacers, extracellular domains, Name: Linker 25
GGGGSGGGGSGGGGS

SEQ ID NO: 42 - protein, 143 aa, synthetic construct
  REGION 1..143: note = Description of sequence: Kind: Linkers, Hinge, spacers, extracellular domains, Name: mouse Thy1.1 spacer and GPI transfer signal
QKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 43 - protein, 111 aa, synthetic construct
  REGION 1..111: note = Description of sequence: Kind: Linkers, Hinge, spacers, extracellular domains, Name: Thy1.1 spacer
QKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKC

SEQ ID NO: 44 - protein, 312 aa, synthetic construct
  REGION 1..312: note = Description of sequence: Kind: Linkers, Hinge, spacers, extracellular domains, Name: human EGFRt extracellular domain
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 45 - protein, 44 aa, synthetic construct
  REGION 1..44: note = Description of sequence: Kind: Linkers, Hinge, spacers, extracellular domains, Name: mouse CD8 hinge spacer
STTTKPVLRTPSPVHPTGTSQPQRPEDCRPRGSVKGTGLDFACD

SEQ ID NO: 46 - protein, 44 aa, synthetic construct
  REGION 1..44: note = Description of sequence: Kind: Linkers, Hinge, spacers, extracellular domains, Name: mouse CD8 C to S hinge spacer
STTTKPVLRTPSPVHPTGTSQPQRPEDSRPRGSVKGTGLDFASD

SEQ ID NO: 47 - protein, 36 aa, synthetic construct
  REGION 1..36: note = Description of sequence: Kind: Linkers, Hinge, spacers, extracellular domains, Name: mouse CD28 hinge spacer
IEFMYPPPYLDNERSNGTIIHIKEKHLCHTQSSPKL

SEQ ID NO: 48 - protein, 9 aa, synthetic construct
  REGION 1..9: note = Description of sequence: Kind: Linkers, Hinge, spacers, extracellular domains, Name: blocking spacer, CD28
CHTQSSPKL

SEQ ID NO: 49 - protein, 13 aa, synthetic construct
  REGION 1..13: note = Description of sequence: Kind: Linkers, Hinge, spacers, extracellular domains, Name: blocking spacer, CD28
VPRDCGCKPCICT

SEQ ID NO: 50 - protein, 12 aa, synthetic construct
  REGION 1..12: note = Description of sequence: Kind: Linkers, Hinge, spacers, extracellular domains, Name: blocking spacer, CD28
CPSPLFPGPSKP

SEQ ID NO: 51 - protein, 14 aa, synthetic construct
  REGION 1..14: note = Description of sequence: Kind: Linkers, Hinge, spacers, extracellular domains, Name: blocking spacer, CD28
EPKSCDKTHTCPPC

SEQ ID NO: 52 - protein, 145 aa, synthetic construct
  REGION 1..145: note = Description of sequence: Kind: Linkers, Hinge, spacers, extracellular domains, Name: mouse PD-1 extracellular domain
LEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGM

SEQ ID NO: 53 - protein, 25 aa, synthetic construct
  REGION 1..25: note = Description of sequence: Kind: Linkers, Hinge, spacers, extracellular domains, Name: mouse PD-1 hinge
TERILETSTRYPSPSPKPEGRFQGM

SEQ ID NO: 54 - protein, 213 aa, synthetic construct
  REGION 1..213: note = Description of sequence: Kind: Linkers, Hinge, spacers, extracellular domains, Name: mouse CD200R1 extracellular domain
TDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHWEQNNVSDVSCIVSHLTGNQSLSIELSRGGNQSLRP

SEQ ID NO: 55 - protein, 148 aa, synthetic construct
  REGION 1..148: note = Description of sequence: Kind: Linkers, Hinge, spacers, extracellular domains, Name: mouse Fas extracellular domain
QGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNR

SEQ ID NO: 56 - protein, 27 aa, synthetic construct
  REGION 1..27: note = Description of sequence: Kind: Transmembrane domains, Name: mouse CD28
FWALVVVAGVLFCYGLLVTVALCVIWT

SEQ ID NO: 57 - protein, 27 aa, synthetic construct
  REGION 1..27: note = Description of sequence: Kind: Transmembrane domains, Name: mouse CD28
FWALVVVAGVLFCYGLLVTVALCVIWT

SEQ ID NO: 58 - protein, 25 aa, synthetic construct
  REGION 1..25: note = Description of sequence: Kind: Transmembrane domains, Name: mouse CD8
IYIWAPLAGICVALLLSLIITLICY

SEQ ID NO: 59 - protein, 21 aa, synthetic construct
  REGION 1..21: note = Description of sequence: Kind: Transmembrane domains, Name: mouse CD8
IYIWAPLAGTCGVLLLSLVIT

SEQ ID NO: 60 - protein, 23 aa, synthetic construct
  REGION 1..23: note = Description of sequence: Kind: Transmembrane domains, Name: mouse CD4
VFLACVLGGSFGFLGFLGLCILC

SEQ ID NO: 61 - protein, 25 aa, synthetic construct
  REGION 1..25: note = Description of sequence: Kind: Transmembrane domains, Name: mouse OX40
AFAVLLGLGLGLLAPLTVLLALYLL

SEQ ID NO: 62 - protein, 21 aa, synthetic construct
  REGION 1..21: note = Description of sequence: Kind: Transmembrane domains, Name: mouse CD27
IFVTFSSMFLIFVLGAILFFH

SEQ ID NO: 63 - protein, 17 aa, synthetic construct
  REGION 1..17: note = Description of sequence: Kind: Transmembrane domains, Name: mouse Fas
LWLLTILVLLIPLVFIY

SEQ ID NO: 64 - protein, 29 aa, synthetic construct
  REGION 1..29: note = Description of sequence: Kind: Cytoplasmic (intracellular) domains, Name: mouse CD3z delta all Y to F
RAKFSRSAETAANLQDTFDALHMQTLAPR

SEQ ID NO: 65 - protein, 29 aa, synthetic construct
  REGION 1..29: note = Description of sequence: Kind: Cytoplasmic (intracellular) domains, Name: mouse CD3z delta residual Y
RAKFSRSAETAANLQDTYDALHMQTLAPR

SEQ ID NO: 66 - protein, 113 aa, synthetic construct
  REGION 1..113: note = Description of sequence: Kind: Cytoplasmic (intracellular) domains, Name: mouse CD3 zeta wild type
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 67 - protein, 113 aa, synthetic construct
  REGION 1..113: note = Description of sequence: Kind: Cytoplasmic (intracellular) domains, Name: mouse CD3 1XX
RAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPR

SEQ ID NO: 68 - protein, 41 aa, synthetic construct
  REGION 1..41: note = Description of sequence: Kind: Cytoplasmic (intracellular) domains, Name: mouse CD28
NSRRNRLLQSDYMNMTPRRPGLTRKPYQPYAPARDFAAYRP

SEQ ID NO: 69 - protein, 42 aa, synthetic construct
  REGION 1..42: note = Description of sequence: Kind: Cytoplasmic (intracellular) domains, Name: human 4-1BB
KRGRKKLLYIFKQPFMRPVQTTQEEDGCSCRFPEEEEGGCEL

SEQ ID NO: 70 - protein, 36 aa, synthetic construct
  REGION 1..36: note = Description of sequence: Kind: Cytoplasmic (intracellular) domains, Name: mouse OX40
RKAWRLPNTPKPCWGNSFRTPIQEEHTDAHFTLAKI

SEQ ID NO: 71 - protein, 47 aa, synthetic construct
  REGION 1..47: note = Description of sequence: Kind: Cytoplasmic (intracellular) domains, Name: mouse CD27
QRRNHGPNEDRQAVPEEPCPYSCPREEEGSAIPIQEDYRKPEPAFYP

SEQ ID NO: 72 - protein, 239 aa, synthetic construct
  REGION 1..239: note = Description of sequence: Kind: Antigen binding domains, Name: 1D3 CD19 scFv - VH G4S3 VL
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKR

SEQ ID NO: 73 - protein, 116 aa, synthetic construct
  REGION 1..116: note = Description of sequence: Kind: Antigen binding domains, Name: 334354 hCD19 single domain human heavy chain VHH - Patent: WO2020018922A1
EVQLVESGGGLVQPGGSLRLSCAASGFSFSDFWMSWVRQAPGKGLEWVATISQAGSEKDYVDSVKGRFTISRDNAKKSLYLQMNSLRAEDTAVYYCASGVYSFDYRGQGTLVTVSS

SEQ ID NO: 74 - protein, 243 aa, synthetic construct
  REGION 1..243: note = Description of sequence: Kind: Antigen binding domains, Name: 18B12 CD20 scFv - VL G4S3 VH - Patent: WO2007064911A1
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSS

SEQ ID NO: 75 - protein, 123 aa, synthetic construct
  REGION 1..123: note = Description of sequence: Kind: Antigen binding domains, Name: 2MC57 CD20 VHH - Patent: US20190092871A1
QVQLQESGGGLVQAGGSLRLSCAASGRDFATYSMAWFRQAPGKERESVATISWSGQRTRYADSVKGRFTISRDNAKNTVYLQMNSLKPEDTAVYYCAMPRTWGEFPPTQYDSWGQGTQVTVSS

SEQ ID NO: 76 - protein, 246 aa, synthetic construct
  REGION 1..246: note = Description of sequence: Kind: Antigen binding domains, Name: HM79-16 CD79b scFv - VL G4S VH
QSILTQPPSISESLGSTARLTCTLRSGISVGNKNIYWYQQMAGSAPRLFLYFFSNSDKELGPGVPNRVSGSKDTSKNAANLQISDLQVEDEAVYYCSIYESNAYVFGSGTQLTVLGGGGSGGGGSGGGGSEVRLLESGGGLVKPEGSLKLSCVASGFTFSDYFMSWVRQAPGKGLEWVAHIYTKSYNYATYYSGSVKGRFTISRDDSRRMLYLQMSNLRTEDTATYYCMDGSYGYWGQGTMVTVSS

SEQ ID NO: 77 - protein, 248 aa, synthetic construct
  REGION 1..248: note = Description of sequence: Kind: Antigen binding domains, Name: CB3s BAFF-R scFv - VL G4S VH - Patent: US20080181888A1
DIQMTQSPSSLSASVGDRVTITCRASQDVSTAVAWYQQKPGKAPKLLIYSASFLYSGVPSRFSGSGSGTDFTLTISSLQPEDFATYYCQQSQISPPTFGQGTKVEIKRGGGGSGGGGSGGGGSEVQLVESGGGLVQPGGSLRLSCAASGFTISSSSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 78 - protein, 10 aa, synthetic construct
  REGION 1..10: note = Description of sequence: Kind: Affinity tags, Name: Myc
EQKLISEEDL

SEQ ID NO: 79 - protein, 41 aa, synthetic construct
  REGION 1..41: note = Description of sequence: Kind: Affinity tags, Name: human CD34 tandem epitope Q2
ELPTQGTFSNVSTNVSGGGGSGGGSELPTQGTFSNVSTNVS

SEQ ID NO: 80 - protein, 9 aa, synthetic construct
  REGION 1..9: note = Description of sequence: Kind: Affinity tags, Name: hCD20 mimotope
CPYSNPSLC

SEQ ID NO: 81 - protein, 31 aa, synthetic construct
  REGION 1..31: note = Description of sequence: Kind: Affinity tags, Name: hCD20 mimotope R2
CPYSNPSLCSGGGGSSGGGGSDCPYSNPSLC

SEQ ID NO: 82 - protein, 8 aa, synthetic construct
  REGION 1..8: note = Description of sequence: Kind: Affinity tags, Name: FLAG
DYKDDDDK

SEQ ID NO: 83 - protein, 14 aa, synthetic construct
  REGION 1..14: note = Description of sequence: Kind: Affinity tags, Name: V5
GKPIPNPLLGLDST

SEQ ID NO: 84 - protein, 28 aa, synthetic construct
  REGION 1..28: note = Description of sequence: Kind: Affinity tags, Name: twin Strep tag
WSHPQFEKGGGSGGGSGGSAWSHPQFEK

SEQ ID NO: 85 - protein, 279 aa, synthetic construct
  REGION 1..279: note = Description of sequence: Kind: Inhibitory ligands, Name: FasL
MQQPMNYPCPQIFWVDSSATSSWAPPGSVFPCPSCGPRGPDQRRPPPPPPPVSPLPPPSQPLPLPPLTPLKKKDHNTNLWLPVVFFMVLVALVGMGLGMYQLFHLQKELAELREFTNQSLKVSSFEKQIANPSTPSEKKEPRSVAHLTGNPHSRSIPLEWEDTYGTALISGVKYKKGGLVINETGLYFVYSKVYFRGQSCNNQPLNHKVYMRNSKYPEDLVLMEEKRLNYCTTGQIWAHSSYLGAVFNLTSADHLYVNISQLSLINFEESKTFFGLYKL

SEQ ID NO: 86 - protein, 290 aa, synthetic construct
  REGION 1..290: note = Description of sequence: Kind: Inhibitory ligands, Name: PD-L1
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 87 - protein, 278 aa, synthetic construct
  REGION 1..278: note = Description of sequence: Kind: Inhibitory ligands, Name: CD200
MGSLVFRRPFCHLSTYSLIWGMAAVALSTAQVEVVTQDERKALHTTASLRCSLKTSQEPLIVTWQKKKAVSPENMVTYSKTHGVVIQPAYKDRINVTELGLWNSSITFWNTTLEDEGCYMCLFNTFGSQKVSGTACLTLYVQPIVHLHYNYFEDHLNITCSATARPAPAISWKGTGTGIENSTESHFHSNGTTSVTSILRVKDPKTQVGKEVICQVLYLGNVIDYKQSLDKGFWFSVPLLLSIVSLVILLILISILLYWKRHRNQERGESSQGMQRMK

SEQ ID NO: 88 - protein, 400 aa, synthetic construct
  REGION 1..400: note = Description of sequence: Kind: iCaspase9, Name: iCasp9
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVD

SEQ ID NO: 89 - protein, 236 aa, synthetic construct
  REGION 1..236: note = Description of sequence: Kind: BCL-2, Name: mouse BCL-2 D34A
MAQAGRTGYDNREIVMKYIHYKLSQRGYEWDAGAADAAPLGAAPTPGIFSFQPESNPMPAVHRDMAARTSPLRPLVATAGPALSPVPPVVHLTLRRAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 90 - protein, 235 aa, synthetic construct
  REGION 1..235: note = Description of sequence: Kind: CD4, CD8 polypeptides, Name: CD8 polypeptide
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 91 - protein, 247 aa, synthetic construct
  REGION 1..247: note = Description of sequence: Kind: CD4, CD8 polypeptides, Name: CD8 polypeptide
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 92 - protein, 220 aa, synthetic construct
  REGION 1..220: note = Description of sequence: Kind: CD4, CD8 polypeptides, Name: CD28 polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 93 - protein, 457 aa, synthetic construct
  REGION 1..457: note = Description of sequence: Kind: CD4, CD8 polypeptides, Name: CD4 polypeptide
MCRAISLRRLLLLLLQLSQLLAVTQGKTLVLGKEGESAELPCESSQKKITVFTWKFSDQRKILGQHGKGVLIRGGSPSQFDRFDSKKGAWEKGSFPLIINKLKMEDSQTYICELENRKEEVELWVFKVTFSPGTSLLQGQSLTLTLDSNSKVSNPLTECKHKKGKVVSGSKVLSMSNLRVQDSDFWNCTVTLDQKKNWFGMTLSVLGFQSTAITAYKSEGESAEFSFPLNFAEENGWGELMWKAEKDSFFQPWISFSIKNKEVSVQKSTKDLKLQLKETLPLTLKIPQVSLQFAGSGNLTLTLDKGTLHQEVNLVVMKVAQLNNTLTCEVMGPTSPKMRLTLKQENQEARVSEEQKVVQVVAPETGLWQCLLSEGDKVKMDSRIQVLSRGVNQTVFLACVLGGSFGFLGFLGLCILCCVRCRHQQRQAARMSQIKRLLSEKKTCQCPHRMQKSHNLI

SEQ ID NO: 94 - protein, 458 aa, synthetic construct
  REGION 1..458: note = Description of sequence: Kind: CD4, CD8 polypeptides, Name: CD4 polypeptide
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 95 - protein, 164 aa, synthetic construct
  REGION 1..164: note = Description of sequence: Kind: CD4, CD8 polypeptides, Name: CD3? polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 96 - protein, 188 aa, synthetic construct
  REGION 1..188: note = Description of sequence: Kind: CD4, CD8 polypeptides, Name: CD3? polypeptide
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 97 - protein, 218 aa, synthetic construct
  REGION 1..218: note = Description of sequence: Kind: CD4, CD8 polypeptides, Name: CD28 polypeptide
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 98 - protein, 255 aa, synthetic construct
  REGION 1..255: note = Description of sequence: Kind: CD4, CD8 polypeptides, Name: 4-1BB polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 99 - protein, 277 aa, synthetic construct
  REGION 1..277: note = Description of sequence: Kind: CD4, CD8 polypeptides, Name: OX40 polypeptide
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 100 - protein, 199 aa, synthetic construct
  REGION 1..199: note = Description of sequence: Kind: CD4, CD8 polypeptides, Name: ICOS polypeptide
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 101 - protein, 20 aa, synthetic construct
  REGION 1..20: note = Description of sequence: Kind: 2A peptides, Name: E2A
QCTNYALLKLAGDVESNPGP

SEQ ID NO: 102 - protein, 22 aa, synthetic construct
  REGION 1..22: note = Description of sequence: Kind: 2A peptides, Name: F2A
VKQTLNFDLLKLAGDVESNPGP

SEQ ID NO: 103 - protein, 19 aa, synthetic construct
  REGION 1..19: note = Description of sequence: Kind: 2A peptides, Name: P2A
ATNFSLLKQAGDVEENPGP

SEQ ID NO: 104 - protein, 18 aa, synthetic construct
  REGION 1..18: note = Description of sequence: Kind: 2A peptides, Name: T2A
EGRGSLLTCGDVEENPGP

SEQ ID NO: 105 - protein, 866 aa, synthetic construct
  REGION 1..866: note = Description of sequence: RR12EE345L linker EE12RR345L Thy1.1 P2A CD20 CD8H CD8TM CD28z E2A
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 106 - protein, 860 aa, synthetic construct
  REGION 1..860: note = Description of sequence: RR12EE345L linker EE12RR345L Thy1.1 P2A CD20 CD28H CD28TM CD28z E2A
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 107 - protein, 1388 aa, synthetic construct
  REGION 1..1388: note = Description of sequence: Q2-RR12EE345L P2A iC9 F2A 3xFLAG BCL2 D34A E2A CD19 CD8H CD8TM CD28z
METDTLLLWVLLLWVPGSTGGSGELPTQGTFSNVSTNVSGGGGSGGGSELPTQGTFSNVSTNVSGGGGSGGGSALGLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSGDYKDHDGDYKDHDIDYKDDDDKGSGMAQAGRTGYDNREIVMKYIHYKLSQRGYEWDAGAADAAPLGAAPTPGIFSFQPESNPMPAVHRDMAARTSPLRPLVATAGPALSPVPPVVHLTLRRAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHKGSGRAKRQCTNYALLKLAGDVESNPGPGSLTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKREQKLISEEDLLESSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGTEARL

SEQ ID NO: 108 - protein, 1097 aa, synthetic construct
  REGION 1..1097: note = Description of sequence: Q2-RR12EE345L P2A iC9 F2A CD19 CD8H CD8TM CD28z
METDTLLLWVLLLWVPGSTGGSGELPTQGTFSNVSTNVSGGGGSGGGSELPTQGTFSNVSTNVSGGGGSGGGSALGLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSLTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKREQKLISEEDLLESSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGTEARL

SEQ ID NO: 109 - protein, 1214 aa, synthetic construct
  REGION 1..1214: note = Description of sequence: R2 3N PD-1 DNR P2A CD20 CD28H CD28TM CD28z E2A Fas-DNR
METDTLLLWVLLLWVPGSTGCPYSNPSLCSGGGGSSGGGGSDCPYSNPSLCGGGGSGGGSLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTGSTSGSGKPGSGEGSTKGLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVFLACVLGGSFGFLGFLGLCILCRAKFSRSAETAANLQDTFDALHMQTLAPRGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLEMLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIYRKYRKRKCWKRRQDDPESRTSSRETIPMNASNLSLQKDLGKSTPDTGNENEGQCLE

SEQ ID NO: 110 - protein, 860 aa, synthetic construct
  REGION 1..860: note = Description of sequence: 3N Thy1.1 P2A CD20 CD28H CD28TM CD28z E2A
METDTLLLWVLLLWVPGSTGLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 111 - protein, 588 aa, synthetic construct
  REGION 1..588: note = Description of sequence: Q2-RR12EE345L P2A iC9 F2A
METDTLLLWVLLLWVPGSTGGSGELPTQGTFSNVSTNVSGGGGSGGGSELPTQGTFSNVSTNVSGGGGSGGGSALGLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSL

SEQ ID NO: 112 - protein, 505 aa, synthetic construct
  REGION 1..505: note = Description of sequence: 3N PD-1 CD28TM CD28z (PD-1 CAR)
METDTLLLWVLLLWVPGSTGLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTGSTSGSGKPGSGEGSTKGLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 113 - protein, 1200 aa, synthetic construct
  REGION 1..1200: note = Description of sequence: R2 3N PD-1 DNR P2A CD20 CD28H CD28TM CD28z E2A FasBB
METDTLLLWVLLLWVPGSTGCPYSNPSLCSGGGGSSGGGGSDCPYSNPSLCGGGGSGGGSLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTGSTSGSGKPGSGEGSTKGLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVFLACVLGGSFGFLGFLGLCILCRAKFSRSAETAANLQDTFDALHMQTLAPRGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLEMLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIYKRGRKKLLYIFKQPFMRPVQTTQEEDGCSCRFPEEEEGGCEL

SEQ ID NO: 114 - protein, 1088 aa, synthetic construct
  REGION 1..1088: note = Description of sequence: 3N Thy1.1 P2A CD20 CD28H CD28TM CD28z E2A FasBB
METDTLLLWVLLLWVPGSTGLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLEMLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIYKRGRKKLLYIFKQPFMRPVQTTQEEDGCSCRFPEEEEGGCEL

SEQ ID NO: 115 - protein, 1162 aa, synthetic construct
  REGION 1..1162: note = Description of sequence: Q2-RR12EE345L P2A CD8SP R2 PD-1H delta F2A CD200R1 CD27 E2A CD19 CD8H CD8TM CD28z
METDTLLLWVLLLWVPGSTGGSGELPTQGTFSNVSTNVSGGGGSGGGSELPTQGTFSNVSTNVSGGGGSGGGSALGLEIRAAFLRQRNTALRTEVAELEQEVQRLENEVSQYETRYGPLGGGKGSGRAKRATNFSLLKQAGDVEENPGPEFTMALPVTALLLPLALLLHAARPCPYSNPSLCSGGGGSSGGGGSDCPYSNPSLCGGGGSGGGSTERILETSTRYPSPSPKPEGRFQGMFWALVVVAGVLFCYGLLVTVALCVIWTRAKFSRSAETAANLQDTFDALHMQTLAPRGTGRAKRVKQTLNFDLLKLAGDVESNPGPGSGMYSMQLASCVTLTLVLLVNSTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHWEQNNVSDVSCIVSHLTGNQSLSIELSRGGNQSLRPIFVTFSSMFLIFVLGAILFFHQRRNHGPNEDRQAVPEEPCPYSCPREEEGSAIPIQEDYRKPEPAFYPGSGQCTNYALLKLAGDVESNPGPGSLTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKREQKLISEEDLLESSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGTEARL

SEQ ID NO: 116 - protein, 1211 aa, synthetic construct
  REGION 1..1211: note = Description of sequence: R2 3N PD-1 OX40 P2A CD20 CD28H CD28TM CD28z E2A FasBB
METDTLLLWVLLLWVPGSTGCPYSNPSLCSGGGGSSGGGGSDCPYSNPSLCGGGGSGGGSLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTGSTSGSGKPGSGEGSTKGTSLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMAFAVLLGLGLGLLAPLTVLLALYLLRKAWRLPNTPKPCWGNSFRTPIQEEHTDAHFTLAKIGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLEMLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIYKRGRKKLLYIFKQPFMRPVQTTQEEDGCSCRFPEEEEGGCEL

SEQ ID NO: 117 - protein, 1240 aa, synthetic construct
  REGION 1..1240: note = Description of sequence: Q2-RR12EE345L P2A R2 BAFF-R R2 CD28H CD28TM CD28 1XX E2A CD79b FLAG PD-1H CD8TM CD28 1XX
METDTLLLWVLLLWVPGSTGGSGELPTQGTFSNVSTNVSGGGGSGGGSELPTQGTFSNVSTNVSGGGGSGGGSALGLEIRAAFLRQRNTALRTEVAELEQEVQRLENEVSQYETRYGPLGGGKGSGRAKRATNFSLLKQAGDVEENPGPGSLTMASPLTRFLSLNLLLLGESIILGSGEACPYSNPSLCSGGGGSSGGGGSDCPYSNPSLCGGGGSGGGSDIQMTQSPSSLSASVGDRVTITCRASQDVSTAVAWYQQKPGKAPKLLIYSASFLYSGVPSRFSGSGSGTDFTLTISSLQPEDFATYYCQQSQISPPTFGQGTKVEIKRGGGGSGGGGSGGGGSEVQLVESGGGLVQPGGSLRLSCAASGFTISSSSIHWVRQAPGKGLEWVAWVLPSVGFTDYADSVKGRFTISADTSKNTAYLQMNSLRAEDTAVYYCARRVCYNRLGVCAGGMDYWGQGTLVTVSSSGGGGSDCPYSNPSLCSGGGGSDCPYSNPSLCGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGSGQCTNYALLKLAGDVESNPGPLEGMYSMQLASCVTLTLVLLVNSQSILTQPPSISESLGSTARLTCTLRSGISVGNKNIYWYQQMAGSAPRLFLYFFSNSDKELGPGVPNRVSGSKDTSKNAANLQISDLQVEDEAVYYCSIYESNAYVFGSGTQLTVLGGGGSGGGGSGGGGSEVRLLESGGGLVKPEGSLKLSCVASGFTFSDYFMSWVRQAPGKGLEWVAHIYTKSYNYATYYSGSVKGRFTISRDDSRRMLYLQMSNLRTEDTATYYCMDGSYGYWGQGTMVTVSSAAADYKDDDDKTERILETSTRYPSPSPKPEGRFQGMALGIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPR

SEQ ID NO: 118 - protein, 866 aa, synthetic construct
  REGION 1..866: note = Description of sequence: 3N Thy1.1 P2A CD20 CD8H CD8TM CD28 1XX
METDTLLLWVLLLWVPGSTGLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGSGQCTNYALLKLAGDVESNPGPLE

SEQ ID NO: 119 - protein, 1217 aa, synthetic construct
  REGION 1..1217: note = Description of sequence: R2 3N PD-1 OX40 P2A CD20 CD8H CD8TM CD28 1XX E2A FasBB
METDTLLLWVLLLWVPGSTGCPYSNPSLCSGGGGSSGGGGSDCPYSNPSLCGGGGSGGGSLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTGSTSGSGKPGSGEGSTKGTSLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMAFAVLLGLGLGLLAPLTVLLALYLLRKAWRLPNTPKPCWGNSFRTPIQEEHTDAHFTLAKIGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGSGQCTNYALLKLAGDVESNPGPLEMLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIYKRGRKKLLYIFKQPFMRPVQTTQEEDGCSCRFPEEEEGGCEL

SEQ ID NO: 120 - protein, 1091 aa, synthetic construct
  REGION 1..1091: note = Description of sequence: 334354 hCD19 VHH myc CD8H CS CD8TM CD28 1XX E2A 2MC57 VHH CD20 ST CD8H CD8TM CD28 1XX P2A V5 3N PD-1H delta
MYSMQLASCVTLTLVLLVNSEVQLVESGGGLVQPGGSLRLSCAASGFSFSDFWMSWVRQAPGKGLEWVATISQAGSEKDYVDSVKGRFTISRDNAKKSLYLQMNSLRAEDTAVYYCASGVYSFDYRGQGTLVTVSSEQKLISEEDLAAASTTTKPVLRTPSPVHPTGTSQPQRPEDSRPRGSVKGTGLDFASDALGIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGSGQCTNYALLKLAGDVESNPGPLEGMASPLTRFLSLNLLLLGESIILGSGEAQVQLQESGGGLVQAGGSLRLSCAASGRDFATYSMAWFRQAPGKERESVATISWSGQRTRYADSVKGRFTISRDNAKNTVYLQMNSLKPEDTAVYYCAMPRTWGEFPPTQYDSWGQGTQVTVSSSAWSHPQFEKGGGSGGGSGGSAWSHPQFEKGGGGSGGGSEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGSGATNFSLLKQAGDVEENPGPTRSMETDTLLLWVLLLWVPGSTGGSGGKPIPNPLLGLDSTGGGGSGGGSLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKGGGGSGGGSTERILETSTRYPSPSPKPEGRFQGMFWALVVVAGVLFCYGLLVTVALCVIWTRAKFSRSAETAANLQDTFDALHMQTLAPR

SEQ ID NO: 121 - protein, 1319 aa, synthetic construct
  REGION 1..1319: note = Description of sequence: 334354 hCD19 VHH myc CD8H CS CD8TM CD28 1XX E2A 2MC57 VHH CD20 ST CD8H CD8TM CD28 1XX P2A R2 3N CD200R1 CD27
MYSMQLASCVTLTLVLLVNSEVQLVESGGGLVQPGGSLRLSCAASGFSFSDFWMSWVRQAPGKGLEWVATISQAGSEKDYVDSVKGRFTISRDNAKKSLYLQMNSLRAEDTAVYYCASGVYSFDYRGQGTLVTVSSEQKLISEEDLAAASTTTKPVLRTPSPVHPTGTSQPQRPEDSRPRGSVKGTGLDFASDALGIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGSGQCTNYALLKLAGDVESNPGPLEGMASPLTRFLSLNLLLLGESIILGSGEAQVQLQESGGGLVQAGGSLRLSCAASGRDFATYSMAWFRQAPGKERESVATISWSGQRTRYADSVKGRFTISRDNAKNTVYLQMNSLKPEDTAVYYCAMPRTWGEFPPTQYDSWGQGTQVTVSSSAWSHPQFEKGGGSGGGSGGSAWSHPQFEKGGGGSGGGSEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGSGATNFSLLKQAGDVEENPGPTRSMETDTLLLWVLLLWVPGSTGCPYSNPSLCSGGGGSSGGGGSDCPYSNPSLCGGGGSGGGSLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTGSTSGSGKPGSGEGSTKGTSTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHWEQNNVSDVSCIVSHLTGNQSLSIELSRGGNQSLRPIFVTFSSMFLIFVLGAILFFHQRRNHGPNEDRQAVPEEPCPYSCPREEEGSAIPIQEDYRKPEPAFYP

SEQ ID NO: 122 - protein, 491 aa, synthetic construct
  REGION 1..491: note = Description of sequence: IL-2SP PD-1 OX40 P2A FasBB
MYSMQLASCVTLTLVLLVNSTSLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMAFAVLLGLGLGLLAPLTVLLALYLLRKAWRLPNTPKPCWGNSFRTPIQEEHTDAHFTLAKIGSGATNFSLLKQAGDVEENPGPGSLTRAKRGSGLEMLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIYKRGRKKLLYIFKQPFMRPVQTTQEEDGCSCRFPEEEEGGCEL

SEQ ID NO: 123 - RNA, 20 nt, synthetic construct
  misc_feature 1..20: note = Description of sequence: CD38 gRNA
  source 1..20: mol_type = other RNA
taaattcatagttagccatt

SEQ ID NO: 124 - RNA, 20 nt, synthetic construct
  misc_feature 1..20: note = Description of sequence: CD19 gRNA
  source 1..20: mol_type = other RNA
tgattcaaactgctcccccg

SEQ ID NO: 125 - RNA, 20 nt, synthetic construct
  misc_feature 1..20: note = Description of sequence: CD22 gRNA
  source 1..20: mol_type = other RNA
tgtcattggcacgtatcggg

SEQ ID NO: 126 - DNA, 141 bp, synthetic construct
  misc_feature 1..141: note = Description of sequence: RR12EE345L zipper
  source 1..141: mol_type = other DNA
ctggagatccgcgctgcatttttgcgacaaagaaataccgctttgcgaaccgaagtggccgagttggagcaggaagtacaacgcctggaaaatgaagtcagccaatacgaaactcgatatggacctctcggaggtggaaag

SEQ ID NO: 127 - DNA, 141 bp, synthetic construct
  misc_feature 1..141: note = Description of sequence: RR12EE345L zipper
  source 1..141: mol_type = other DNA
ctggagatcgaagctgcatttttgcgacaaagaaataccgctttgcgaaccgaagtggccgagttggagcaggaagtacaacgcctggaaaatgaagtcagccaatacgaaactcgatatggacctctcggaggtggaaag

SEQ ID NO: 128 - DNA, 141 bp, synthetic construct
  misc_feature 1..141: note = Description of sequence: RR12EE345L zipper
  source 1..141: mol_type = other DNA
ctggagatccgcgctgcatttttgcgacaaagaaataccgctttgcgaacccgcgtggccgagttggagcaggaagtacaacgcctggaaaatgaagtcagccaatacgaaactcgatatggacctctcggaggtggaaag

SEQ ID NO: 129 - DNA, 141 bp, synthetic construct
  misc_feature 1..141: note = Description of sequence: RR12EE345L zipper
  source 1..141: mol_type = other DNA
ctggagatcgaagctgcatttttgcgacaagaaaataccgctttgcgaaccgaagtggccgagttggagcaggaagtacaacgcctggaaaatgaagtcagccaatacgaaactcgatatggacctctcggaggtggaaag

SEQ ID NO: 130 - DNA, 141 bp, synthetic construct
  misc_feature 1..141: note = Description of sequence: RR12EE345L zipper
  source 1..141: mol_type = other DNA
ctggagatccgcgctgcatttttgcgacaaagaaataccgctttgcgaacccgcgtggccgagttggagcagcgcgtacaacgcctggaaaatgaagtcagccaatacgaaactcgatatggacctctcggaggtggaaag

SEQ ID NO: 131 - DNA, 141 bp, synthetic construct
  misc_feature 1..141: note = Description of sequence: RR12EE345L zipper
  source 1..141: mol_type = other DNA
ctggagatcgaagctgcatttttgcgacaagaaaataccgctttgcgaacccgagtggccgagttggagcaggaagtacaacgcctggaaaatgaagtcagccaatacgaaactcgatatggacctctcggaggtggaaag

SEQ ID NO: 132 - DNA, 141 bp, synthetic construct
  misc_feature 1..141: note = Description of sequence: RR12EE345L zipper
  source 1..141: mol_type = other DNA
ctggagatccgcgctgcatttttgcgacaaagaaataccgctttgcgaacccgagtggccgagttggagcagcgagtacaacgcctggaaaatcgagtcagccaatacgaaactcgatatggacctctcggaggtggaaag

SEQ ID NO: 133 - DNA, 141 bp, synthetic construct
  misc_feature 1..141: note = Description of sequence: RR12EE345L zipper
  source 1..141: mol_type = other DNA
ctggagattgaggctgcatttcttgaaagagaaaacactgctcttgagacaagagtcgccgaactgaggcagcgcgttcagcgcctgcggaaccgagtatctcaatacaggactcggtacggaccactggggggcggtaag

(19) United States
(12) Patent Application Publication
(10) Pub. No.: US 2025/0332199 A1
(43) Pub. Date: Oct. 30, 2025

(54) MULTI-SWITCH RECEPTOR ARRAYS AND METHODS FOR IMPROVING IMMUNE CELL FUNCTION

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Scott JAMES, New York, NY (US); Adhithi RAJAGOPALAN, New York, NY (US); Sophia CHEN, New York, NY (US); Marcel VAN DEN BRINK, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 19/258,003

(22) Filed: Jul. 2, 2025

Related U.S. Application Data
(63) Continuation of application No. PCT/US2024/010502, filed on Jan. 5, 2024.
(60) Provisional application No. 63/478,664, filed on Jan. 5, 2023.

(51) Int. Cl. A61K 35/17; A61K 40/11; A61K 40/31; A61K 40/42; A61P 35/02; A61P 37/04; C07K 14/725; C07K 16/28; C12N 5/00; C12N 5/0783; C12N 9/64

(52) U.S. Cl. CPC A61K 35/17; A61K 40/11; A61K 40/31; A61K 40/421; A61P 35/02; A61P 37/04; C07K 14/7051; C07K 16/2803; C07K 16/2818; C07K 16/2878; C12N 5/0018; C12N 5/0636; C12N 9/6472; A61K 2239/13; A61K 2239/17; A61K 2239/21; A61K 2239/22; A61K 2239/25; C07K 2319/20; C07K 2319/73; C12N 2501/727; C12Y 304/22062

(57) ABSTRACT

The present disclosure is directed to leucine zipper-based sorting systems adapted to facilitate the expression and coordination of polypeptide sequences capable of improving the function of CAR T cells. The systems enable the generation of T cells engineered to express multiple combinations of CARs (multi-CAR), safety-switches, switch receptors, and/or cytokines.

### CROSS-REFERENCE TO RELATED APPLICATIONS

This application is a Continuation of International Patent Application No. PCT/US2024/010502 filed Jan. 5, 2024, which claims priority to U.S. Provisional Patent Application No. 63/478,664, filed on Jan. 5, 2023, the entire contents of which are incorporated by reference herein.

### SEQUENCE LISTING

The specification further incorporates by reference the Sequence Listing submitted herewith via EFS on Jul. 2, 2025. Pursuant to 37 C.F.R. § 1.52(e)(5), the Sequence Listing XML file, identified as 0727341775_ST26.xml, is 202,547 bytes and was created on Jul. 1, 2025. The Sequence Listing, electronically filed herewith, does not extend beyond the scope of the specification and thus does not contain new matter.

### BACKGROUND OF THE INVENTION

Chimeric antigen receptor (CAR) T cell therapy has demonstrated remarkable therapeutic activity in refractory B cell malignancies and myeloma. However, CAR T cells targeting both hematologic malignancies and solid tumors face a number of challenges that limit their safety and efficacy including antigen-loss or antigen-low escape of malignant cells; T cell exhaustion related to tonic CAR signaling and repetitive antigen-stimulation; immunosuppressive tumor microenvironments; lack of target antigen specificity for tumor cells; and CAR T cell-mediated cytokine release syndrome (CRS) and neurotoxicity. Synthetic biology approaches have produced multiple solutions to address these problems individually. For example, engineered co-expression of multiple CARs can overcome escape of antigen-low/negative disease. In addition, temporal manipulation of CAR expression or activation, regulation of CAR expression density, attenuation of CAR CD3ζ signal strength, and 4-1BB costimulation can attenuate tonic-signaling and antigen-stimulation-induced T cell dysfunction. Dominant negative and switch receptors blocking PD-1, CD200R1, and Fas can also improve CAR T cell activity in response to tumor-mediated immune suppression.

As tumor cells utilize many immune evasion strategies, CAR T cells may fail due to both exhaustion and tumor-mediated suppression. A combination of multiple synthetic biology solutions may be required to enhance CAR T cell activity. There are, however, challenges to coalescing multiple strategies due to limitations in packaging and delivery of large vector inserts encoding multiple transgenes. There is, therefore, a need in the art for systems and methodologies in disease treatment that improve the efficacy of T cells and CAR T cells by efficiently networking multiple CARs, safety switches, switch receptors, and/or cytokines.

### SUMMARY OF THE INVENTION

The presently disclosed subject matter is directed, in certain embodiments, to leucine zipper-based sorting systems adapted to facilitate the expression and coordination of polypeptide sequences capable of improving the function of immune cells. In certain embodiments, the systems facilitate the generation of immune cells engineered to express multiple combinations of CARs (multi-CAR), safety-switches, switch receptors, and/or cytokines. For example, but not by way of limitation, the present disclosure is directed to systems comprising a plurality of nucleic acid constructs, wherein the plurality comprises:
(a) a first nucleic acid construct encoding a membrane bound polypeptide comprising:
   (i) an extracellular domain comprising a first leucine zipper sequence;
   (ii) a transmembrane domain; and
   (iii) an intracellular domain;
(b) a second nucleic acid construct encoding a soluble polypeptide comprising a second leucine zipper sequence capable of heterodimerizing with the first leucine zipper sequence and a signal peptide sequence;
   wherein each of the first and second nucleic acid constructs further encode one or more CAR, safety switch, switch receptor, and/or cytokine.

In certain embodiments of the presently disclosed subject matter, one or more of the membrane bound polypeptide, the one or more CAR, safety switch, and/or switch receptor comprise an immune checkpoint molecule extracellular, transmembrane, or intracellular domain. In certain embodiments, the immune checkpoint molecule is programmed cell death protein 1 (PD-1), Fas, CD200R1, T cell immunoglobulin and ITIM domain (TIGIT), ICOS, cytotoxic T lymphocyte-associated antigen-4 (CTLA-4), B- and T-lymphocyte attenuator (BTLA), T cell immunoglobulin and mucin-domain containing-3 (TIM-3), lymphocyte activation gene-3 (LAG-3), leukocyte associated immunoglobulin like receptor (LAIR1), herpesvirus entry mediator (HVEM), 2B4 (CD244), CD160, Galectin9, V-domain Ig suppressor of T cell activation (VISTA), or P-selectin glycoprotein ligand-1 (PSGL-1). In certain embodiments, the immune checkpoint molecule:
(a) is PD-1 having an extracellular domain set forth in SEQ ID NO: 52;
(b) is CD200R1 having an extracellular domain set forth in SEQ ID NO: 54; and/or
(c) is Fas having an extracellular domain set forth in SEQ ID NO: 55.
   In certain embodiments, the immune checkpoint molecule is a dominant negative variant thereof. In certain embodiments, the dominant negative receptor selected from PD-1-DNR, Fas-DNR, CD200R1-DNR, TIGIT-DNR, ICOS-DNR, CTLA-4-DNR, BTLA-DNR, TIM-3-DNR, LAG-3-DNR, LAIR1-DNR, HVEM-DNR, CD244-DNR, CD160-DNR, VISTA-DNR, PSGL-1-DNR, variants thereof, or combinations thereof.

In certain embodiments of the presently disclosed subject matter, the transmembrane domain of one or more of the membrane bound polypeptide, the one or more CAR, safety switch, and/or switch receptor comprises an amino acid sequence set forth in any one of SEQ ID NOs: 56-63.

In certain embodiments of the presently disclosed subject matter, the intracellular domain of one or more of the membrane bound polypeptide, the one or more CAR, safety switch, and/or switch receptor comprises: a CD3 domain, a costimulatory domain, a suicide gene product, survival gene product, fragments thereof, or combinations thereof. In certain embodiments, the suicide gene product is an inducible Caspase 9 polypeptide (iCasp9). In certain embodiments, the iCasp9 has an amino acid sequence set forth in SEQ ID NO: 88. In certain embodiments, the survival gene product is a caspase resistant Bcl-2. In certain embodiments, the Bcl-2 has an amino acid sequence set forth in SEQ ID NO: 89.

In certain embodiments of the presently disclosed subject matter, the intracellular domain of one or more of the membrane bound polypeptide, the one or more CAR, safety switch, and/or switch receptor comprises a member of the TNFR super family. In certain embodiments, the intracellular domain comprises an intracellular domain of OX40, CD40, CD30, 4-1BB, CD27, RANK, GITR, LTBR, HVEM, BAFF-R, TACI, BCMA, TROY fragments thereof, or combinations thereof.

In certain embodiments of the presently disclosed subject matter, the system encodes a switch receptor selected from PD-1-OX40, Fas-4-1BB (FasBB), CD200-CD27, TIGIT-4-1BB (TIGITBB), ICOS-CD27, variants thereof, or combinations thereof.

In certain embodiments of the presently disclosed subject matter, the intracellular domain of one or more of the membrane bound polypeptide, the one or more CAR, safety switch, and/or switch receptor lacks a signaling domain.

In certain embodiments of the presently disclosed subject matter, the first nucleic acid construct further comprises a rituximab-binding mimotope tag.

In certain embodiments of the presently disclosed subject matter, the second nucleic acid construct further comprises an enrichment tag selected from the group consisting of CD34 tag (Q2 tag), His tag, Myc-tag, Hemagglutinin (HA)-tag, Flag tag, V5 tag, and T7 tag.

In certain embodiments of the presently disclosed subject matter, the first nucleic acid construct encodes an amino acid sequence set forth in any one of SEQ ID NOs: 1 or 2. In certain embodiments, the first nucleic acid construct encodes a 3N blocked capture zipper. In certain embodiments, the first nucleic acid construct encodes an amino acid sequence set forth in any one of SEQ ID NOs: 3 or 4.

In certain embodiments of the presently disclosed subject matter, the extracellular domain of one or more of the membrane bound polypeptide, the one or more CAR, safety switch, and/or switch receptor further comprises:
a) a linker; and/or
b) a spacer/hinge domain between the extracellular domain and the transmembrane domain.
   In certain embodiments, the linker has an amino acid sequence set forth in any one of SEQ ID NOs: 17-41;
   the spacer/hinge domain has an amino acid sequence set forth in any one of SEQ ID NOs: 42, 43-51, or 53. In certain embodiments, the transmembrane domain has an amino acid sequence set forth in any one of SEQ ID NOs: 56-63.

In certain embodiments of the presently disclosed subject matter, each CAR comprises an antigen binding domain, a transmembrane domain, and an intracellular signaling domain. In certain embodiments of the presently disclosed subject matter, each CAR binds to a cell surface antigen selected from, CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, BAFF-R, CD79, PD-1, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, ZDHHC11, variants thereof, or combinations thereof. In certain embodiments, each CAR binds to a cell surface antigen selected from BAFF-R, CD79, CD19, CD20, CD70, PD-1, Tim-3, or variants thereof.

In certain embodiments of the presently disclosed subject matter, the system comprises one or more nucleic acid constructs encoding a sequence set forth in any one of SEQ ID NO: 105-122.

In certain embodiments, the presently disclosed subject matter is directed to an engineered immune cell comprising a system as described herein. In certain embodiments, the engineered immune cell is a T cell.

In certain embodiments, the presently disclosed subject matter is directed to method of modifying a cell comprising delivering to the cell, a system as described herein. In certain embodiments, the cell is a mammalian cell. In certain embodiments, the mammalian cell is an immune cell. In certain embodiments, the immune cell is a T cell.

In certain embodiments, the presently disclosed subject matter is directed to methods for enriching a population of modified cells comprising: (a) delivering to the cell, a system of the present disclosure to obtain a population of cells comprising modified cells; (b) culturing the population of cells; and (c) enriching for the population of modified cells by selecting for expression of the enrichment tag.

1. In certain embodiments, the presently disclosed subject matter is directed to methods for improving T cell function comprising delivering to the T cell, a system of the present disclosure to obtain a modified T cell, wherein the modified T cell exhibits at least one characteristic selected from enhanced proliferation, enhanced survival, enhanced persistence, enhanced activation, and reduced exhaustion compared to a control, unmodified T cell. In certain embodiments, the modified population of T cells exhibits attenuated signaling in response to PD-1, Fas, CD200R1, TIGIT, ICOS, CTLA-4, BTLA, TIM-3, LAG-3, LAIR1, HVEM, 2B4 (CD244), CD160, Galectin9, VISTA, and/or PSGL-1.
2. specific ligands, compared to the population of T cells before the delivering step. In certain embodiments, the modified population of T cells exhibits enhanced OX40, CD40, CD30, 4-1BB, CD27, RANK, GITR, LTBR, HVEM, BAFF-R, TACI, BCMA, and/or TROY signaling in response to binding of one or more of PD-1, Fas, CD200R1, TIGIT, ICOS, CTLA-4, BTLA, TIM-3, LAG-3, LAIR1, HVEM, 2B4 (CD244), CD160, Galectin9, VISTA, and/or PSGL-1 specific ligands, compared to the population of T cells before the delivering step.
3. In certain embodiments, the presently disclosed subject matter is directed to methods of treating a disease comprising administering to a subject in need thereof: (a) a population of modified cells comprising a system of the present disclosure; (b) a cell modified according to the method of the present disclosure; and/or (c) an enriched population of cells according to a method of the present disclosure. In certain embodiments, the methods further comprise, culturing the T cells with a small molecular weight inhibitor before administering to the subject. In certain embodiments, the small molecular weight inhibitor is a Src kinase inhibitor. In certain embodiments, the Src inhibitor is Dasatinib. In certain embodiments, the subject is a human subject. In certain embodiments, the disease is a cancer, an autoimmune disease, an inflammatory disease, or a graft versus-host disease. In certain embodiments, the cancer is leukemia, lymphoma, myeloma, ovarian cancer, breast cancer, bladder cancer, brain cancer, colon cancer, intestinal cancer, liver cancer, lung cancer, pancreatic cancer, prostate cancer, testicular cancer, anal cancer, skin cancer, stomach cancer, glioblastoma, throat cancer, melanoma, neuroblastoma, adenocarcinoma, glioma, or soft tissue sarcoma. In certain embodiments, the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia. In certain embodiments, the lymphoma is Hodgkin's lymphoma or non-Hodgkin's lymphoma. In certain embodiments, the non-Hodgkin's lymphoma is B-cell non-Hodgkin's lymphoma or T-cell non-Hodgkin's lymphoma. In certain embodiments, the cancer comprises cells expressing BAFF-R, CD79, CD70, CD19, CD20, PD-1, VISTA, PSGL-1, Tim-3, variants thereof or combinations thereof. In certain embodiments, the cancer comprises cells expressing at least one antigen selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, ZDHHC11 variants thereof, or combinations thereof.

### BRIEF DESCRIPTION OF THE DRAWINGS

FIGS. 1A-1E illustrate exemplary leucine zipper sorting systems of the present disclosure for the coordinated expression of multiple CARs, safety switches, switch receptors, and/or cytokines. FIG. 1A illustrates an approach to create dual-vector-transduced cells by enabling single-step magnetic-activated cell sorting (MACS), where the approach utilizes a heterodimerizing leucine zipper pair encoded by two vectors: (1) a secreted affinity-tagged zipper and (2), a membrane-bound capture-zipper. FIG. 1B illustrates the utility of switch receptors to convert inhibitory ligand signals into positive costimulatory signals while simultaneously blocking interactions of these ligands with their native receptors. FIG. 1C illustrates an exemplary dual-CAR and multi-Switch CAR T cell manufactured by transducing the cell with two vectors—vector 1 encoding the tandem-CD34 tag “Q2” secreted leucine zipper, Rituximab-binding safety switch stalk, CD200R1-CD27 switch receptor, and a CD19-CAR; and vector 2 encoding a PD-1-OX40 switch receptor with 3N blocked capture-zipper and Rituximab-binding cyclic CD20 mimotope tag, a CD20-CAR, and FasBB switch receptor. FIG. 1D illustrates vector maps for multi-Switch receptors. FIG. 1E illustrates high purity expression of CARs and switch receptors in primary T cells transduced with the two vectors described in FIGS. 1C and 1D, following Zip-sorting anti-CD34 bead sorting.

FIGS. 2A-2D illustrate an exemplary leucine zipper sorting system of the present disclosure comprising two CARs, two dominant negative receptors (DNR), and caspase-cleavage-resistant BCL2. FIG. 2A illustrates T cells expressing two CARs (e.g., anti-CD19 CAR, anti-CD20 CAR), two DNRs (e.g., Fas-DNR, PD-1-DNR), and caspase-cleavage-resistant BCL2. FIG. 2B vector maps used for generating the engineered T cells. FIG. 2C shows FACS analysis for confirming expression of the indicated elements in the T cell described in FIG. 2A. FIG. 2D shows vector maps for generating capture zipper modified PD-1 receptor based CAR T cells. FIG. 2E shows purification and enrichment of the cells using a CD34 affinity tag. FIG. 2F shows binding of PD-L1 Fc to the 3N blocked capture-zipper modified PD-1 molecule demonstrating absence of steric hindrance of ligand binding resulting from the capture-zipper. FIG. 2G show viability studies for PD-L1+/CD20+ and PD-L1−/CD20+ BM185 target cells when cocultured with PD-1 CAR T cells at different Effector to Target ratios (E:T).

FIGS. 3A-3C illustrate enhanced NFkB activity and expansion in multi-Switch receptor-expressing CAR T cells. FIG. 3A shows NFkB reporter intensity, T cell expansion kinetics and T cell-target cell cluster formation following target encounter. FIG. 3B shows elimination of leukemia target cells by multi-Switch receptor-expressing CAR T cells. FIG. 3C shows protection of dual-CAR triple-Switch T cells from FasL expressed on target cells; and higher proliferation of these T cells when cultured in the presence of target cells expressing PD-L1 and CD200 inhibitory ligands.

FIGS. 4A-4H illustrate that expression of multiple switch receptors inhibits surface expression of inhibitory ligands. FIGS. 4A-4D shows vector maps and FIG. 4E shows a legend for the various vector combinations used for transducing T cells. FIG. 4F shows FACS analysis of target cell dependent CD200 upregulation in dual CAR/FasBB T cells. FIG. 4G shows FACS analysis of target cell dependent CD200 expression in dual CAR/FasBB, dual CAR/dual switch, and dual CAR/triple switch T cells (see FIG. 4E). FIG. 4H provides a quantitative presentation of the data shown in FIG. 4G.

FIGS. 5A-5D illustrate the efficacy of dual-CAR multi-Switch receptor T cells in mediating enhanced clearance of leukemic cells. FIG. 5A shows the results of bioluminescence imaging (BLI) demonstrating clearance of leukemia cells in mice receiving the indicated T cells. FIG. 5B shows the results of survival studies in mice receiving the various indicated T cells. FIG. 5C shows the results of bioluminescence imaging (BLI) demonstrating clearance of leukemia cells in mice receiving the indicated T cells cultured with Dasatinib. FIG. 5D shows the results of survival studies in mice receiving the various indicated T cells cultured with Dasatinib.

FIGS. 6A-6H illustrate use of exemplary dual-Switch receptor configuration in triple-CAR T cells to enhance T cell expansion and leukemia clearance. FIG. 6A is an illustration of the triple-CAR dual-Switch receptor T cells and vector maps. FIG. 6B shows characterization of surface expression of CARs and switch receptors following dual-transduction and Zip-sorting. FIG. 6C shows the ability of triple-CAR T cells to kill targets expressing either CD20, CD79b, or BAFF-R. FIG. 6D show the ability of the triple-CAR T cells to eliminate a mixture of C1498 leukemia expressing CD20, CD79b, and BAFF-R in vivo as demonstrated by bioluminescence imaging of animals. FIG. 6E shows enhanced overall survival of leukemia injected animals receiving the triple-CAR or triple-CAR dual-Switch T cells. FIG. 6F shows greater expansion of triple-CAR dual-Switch T cells in vivo as demonstrated by bioluminescence imaging of animals injected with bioluminescent T cells. FIG. 6G shows enhanced anti-leukemia activity in mice injected triple-CAR dual-Switch T cells as demonstrated by bioluminescence imaging. FIG. 6H shows overall survival of animals treated as described in FIG. 6G.

FIGS. 7A-7H illustrate the use of exemplary switch receptor arrays to enhance the anti-leukemia activity of quad-CAR T cells. FIGS. 7A-7B are illustrations of vectors encoding CD19, CD20, CD79b, BAFF-R CARs and switch receptors. FIG. 7C shows a characterization of surface expression of four CARs and three switch receptors following triple-transduction and Zip-sorting. FIG. 7D shows enhanced T cell expansion (bioluminescent T cells) in mice bearing C1498 leukemia expressing CD19, CD20, CD79b, and BAFF-R and treated with Quad-CAR T cells. FIG. 7E shows the enhanced ability of quad-CAR T cells co-expressing switch receptors to eliminate leukemia in mice as demonstrated by bioluminescence imaging (bioluminescent leukemia cells). FIG. 7F show overall survival of animals treated as described in FIG. 7E.

FIGS. 8A-8D illustrates the use of exemplary compositions where cognate switch-receptor/ligand interactions drive ligand-specific expansion of multi-Switch BCMA-CAR T cells. FIG. 8A illustrates co-expression of multiple switch receptors enabling T cell proliferation and expansion in response to multiple inhibitory ligands. FIG. 8B shows expansion of T cells as measured by live cell microscopy imaging of NFkB reporter-expressing CAR T cells after encountering MOPC315.BM TACKI^{KO} expressing the inhibitory ligand CD155. FIG. 8C shows expansion of T cells as measured by live cell microscopy imaging of NFkB reporter-expressing CAR T cells after encountering MOPC315.BM TACKI^{KO} expressing the inhibitory ligand CD200. FIG. 8D shows expansion of T cells as measured by live cell microscopy imaging of NFkB reporter-expressing CAR T cells after encountering MOPC315.BM TACKI^{KO} expressing the inhibitory ligand PD-L1.

FIGS. 9A-9C illustrate the clearance of BCMA-low multiple myeloma in vivo by dual-Switch receptor-expressing BCMA-CAR T cells. FIG. 9A illustrates the vector elements used for generating the dual-Switch receptor-expressing BCMA-CAR T cells. FIG. 9B shows bioluminescence imaging of myeloma cells in mice treated with the indicated T cells. FIG. 9C shows long-term survival of myeloma challenged animals after treatment with the indicated T cells.

FIGS. 10A-10D illustrate increased antigen-dependent proliferation in Tim-3, CD70-targeting dual-CAR T cells co-expressing multiple switch receptors FIG. 10A illustrates the vector elements used for generating the Tim-3, CD70-targeting dual-CAR T cells co-expressing multiple switch receptors. FIG. 10B shows FACS analysis showing high purity expression of the Tim-3-CAR (Strep Tag) and CD70-CAR (V5) CARs and all three switch receptors. FIG. 10C shows live cell microscopy imaging of NFkB reporter-expressing CAR T cells (GFP+) as a measure of T cell expansion. FIG. 10D shows the efficacy of these CAR T cells in eliminating iRFP713+ tumors.

FIGS. 11A-11D illustrate that co-expression of multiple switch receptors enhances expansion and anti-leukemia activity of CD8+ Tim-3/CD70-dual CAR T cells. FIG. 11A illustrates exemplary vector elements used for generating the CAR T cells. FIG. 11B shows FACS analysis showing high purity expression of the Tim-3-CAR (Strep Tag) and CD70-CAR (V5) CARs and all three switch receptors. FIG. 11C shows live cell microscopy imaging of NFkB reporter-expressing CAR T cells (GFP+) as a measure of T cell expansion. FIG. 11D shows the efficacy of these CAR T cells in eliminating iRFP713+ tumors.

FIGS. 12A-12D illustrate that co-expression of multiple switch receptors can enhance expansion and anti-leukemia activity of CD8+ Tim-3/CD70-dual CAR T cells against leukemia with very low CD70 expression. FIG. 12A illustrates the vector elements used for generating the Tim-3/CD70-dual CAR dual-Switch T cells. FIG. 12B show Flow cytometry analysis of BM185 engineered to express very high and very low CD70. FIG. 12C shows live cell microscopy imaging of NFkB reporter-expressing CAR T cells (GFP+) as a measure of T cell expansion. FIG. 12D shows efficacy of these CAR T cells in eliminating iRFP713+ tumors.

### DETAILED DESCRIPTION

The presently disclosed subject matter relates to a leucine zipper-based system designed to enable single-step immunomagnetic sorting of cells transduced with two or more vectors with the goal of doubling the amount of genetic information delivered and promoting enhanced transgene expression. This platform facilitates generation of T cells expressing combinations of CARs and drug regulated CAR expression systems, safety-switches, dominant negative and switch receptors, modulators of signal transduction pathways, and engineered cytokine secretion. As outlined herein, this system can be used to overcome antigen-loss escape and tumor-associated immune suppression strategies, inhibit CRS, and attenuate tonic CAR signaling-induced dysfunction.

The subject matter of the present disclosure is described with reference to the Figures. It should be understood that numerous specific details, relationships, and methods are set forth in this Detailed Description, Examples, and accompanying Figures to provide a more complete understanding of the subject matter disclosed herein.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other instances “comprising,” “consisting of”, and “consisting essentially of,” the instances or elements presented herein, whether explicitly set forth or not.

For the recitation of numeric ranges herein, each intervening number within the range is explicitly contemplated with the same degree of precision. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein the term “modular polypeptide” refers to any polypeptide comprised of subunits that when combined reconstitute a mature polypeptide. Non-limiting examples of such modular polypeptide include extracellular domain-containing, intracellular domain-containing, and transmembrane domain-containing polypeptides, as well as polypeptides comprising two or more of such domains e.g., CARs and CCRs.

As used herein, a “linker” refers to a functional group (e.g., chemical or polypeptide) that covalently attaches two or more polypeptides or nucleic acids so that they are connected to one another. In certain embodiments, the linker comprises one or more amino acids used to couple two polypeptides together (e.g., to couple V_{H} and V_{L} domains or to couple two dimerization domains). The linker can be usually rich in glycine for flexibility, as well as serine or threonine for solubility.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence operably linked to appropriate nucleic acid sequences necessary for the expression of the coding sequence in a particular host organism. Nucleic acid sequences necessary for expression in prokaryotes usually include a promoter, an operator (optional), and a ribosome binding site, often along with other sequences. Nucleic acid sequences necessary for expression in eukaryotic cells can include, but are not limited to, promoters, enhancers, and termination and polyadenylation signals.

In certain embodiments, nucleic acid molecules useful in the presently disclosed subject matter include nucleic acid molecules that encode an antibody or an antigen-binding fragment thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule.

As used herein, the term “disease” refers to any condition or disorder that damages or interferes with the normal function of a cell, tissue, or organ. Examples of diseases include neoplasia or pathogenic infection of a cell, tissue, or organ.

An “effective amount” (or “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a neoplasia), or otherwise reduce the pathological consequences of the disease (e.g., a neoplasia). The dose comprising an effective amount is generally determined by the physician on a case-by-case basis and making such a determination is within the level of ordinary skill in the art. Several factors are typically taken into account when determining an appropriate dosage to achieve an effective amount. These factors include age, sex and weight of the subject, the condition being treated, the severity of the condition and the form and effective concentration of the cells (e.g., engineered immune cells) administered.

As used herein, the term “neoplasm” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of skin, bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, spinal cord, spleen, stomach, testes, thymus, thyroid, trachea, urogenital tract, ureter, urethra, uterus, and vagina, or a tissue or cell type thereof. Neoplasia include cancers, such as melanoma, sarcomas, carcinomas, or plasmacytomas (malignant tumor of the plasma cells).

As used herein, the term “immunoresponsive cell” refers to a cell that functions in an immune response, and includes a progenitor of such cell, and a progeny of such cell.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” polypeptide is sufficiently free of other materials such that any impurities do not materially affect the biological properties of the polypeptide or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or polypeptide gives rise to essentially one band in an electrophoretic gel. For a polypeptide that can be subjected to modifications, for example, phosphorylation or glycosylation, different modifications may give rise to different isolated polypeptides, which can be separately purified.

As used herein, the term “secreted” refers to a polypeptide that is released from a cell via the secretory pathway through the endoplasmic reticulum, Golgi apparatus, and as a vesicle that transiently fuses at the cell plasma membrane, releasing the polypeptides outside of the cell.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). The antibodies of the invention comprise whole native antibodies, bispecific antibodies; chimeric antibodies; Fab, Fab′, single chain V region fragments (scFv), fusion polypeptides, and unconventional antibodies. In certain embodiments, an antibody is a glycopolypeptide comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, the term “single-chain variable fragment” or “scFv” is a fusion polypeptide of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C terminus of the V_{L}, or the C-terminus of the V_{H} with the N-terminus of the V_{L}.

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

In certain non-limiting embodiments, an intracellular signaling domain of a CAR or a ZipR CAR comprises a CD3ζ polypeptide, which can activate or stimulate a cell (e.g., a cell of the lymphoid lineage, e.g., a T cell). CD3ζ comprises 3 immunoreceptor tyrosine-based activation motifs (ITAMs) and transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain non-limiting embodiments, a CAR or a ZipR CAR can also comprise a spacer/hinge region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and fragments of CD3, a fragment of a CD28 polypeptide, a fragment of a CD8 polypeptide, a variant thereof, or a synthetic spacer sequence.

As used herein, “costimulatory molecules” refer to cell surface molecules other than antigen receptors or their ligands that are required for a response of lymphocytes to antigen. The at least one co-stimulatory signaling region can include a CD28 polypeptide (e.g., intracellular domain of CD28 or a fragment thereof), a 4-1BB polypeptide (e.g., intracellular domain of 4-1BB or a fragment thereof), an OX40 polypeptide (e.g., intracellular domain of OX40 or a fragment thereof), an ICOS polypeptide (e.g., intracellular domain of ICOS or a fragment thereof), a DAP-10 polypeptide (e.g., intracellular domain of DAP10 or a fragment thereof), or a combination thereof. The co-stimulatory molecule can bind to a co-stimulatory ligand. As used herein, the term a “co-stimulatory ligand” refers to a polypeptide expressed on cell surface that upon binding to its receptor produces a co-stimulatory response, i.e., an intracellular response that effects the stimulation provided by an activating signaling domain (e.g., a CD3ζ signaling domain). Non-limiting examples of co-stimulatory ligands include tumor necrosis factor (TNF) family members, immunoglobulin (Ig) superfamily members, or combination thereof. the co-stimulatory ligand is selected from the group consisting of tumor necrosis factor (TNF) family members, immunoglobulin (Ig) superfamily members, and combinations thereof. Non-limiting examples of TNF family member include 4-1BBL, OX40L, CD70, GITRL, CD40L, and CD30L. Non-limiting examples of Ig superfamily member include CD80, CD86, and ICOSLG. For example, 4-1BBL may bind to 4-1BB for providing an intracellular signal that in combination with a CAR signal induces an effector cell function of the CAR⁺ T cell. CARs comprising an intracellular signaling domain that comprises a co-stimulatory signaling region comprising a 4-1BB, ICOS or DAP-10 co-stimulatory signaling domain are disclosed in U.S. Pat. No. 7,446,190, which is herein incorporated by reference in its entirety.

As used herein, the term “multimerization” refers to the formation of multimers (including dimers). Multimerization includes dimerization.

As used herein, the term “a conservative sequence modification” refers to an amino acid modification that does not significantly affect or alter the binding characteristics of the presently disclosed polypeptide (e.g., the extracellular antigen-binding domain of the polypeptide) comprising the amino acid sequence. Conservative modifications can include amino acid substitutions, additions and deletions. Modifications can be introduced into the human scFv of the presently disclosed polypeptide by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively charged amino acids include lysine, arginine, histidine, negatively charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence or a CDR region are altered.

As used herein, “very low” expression of a ligand, receptor, peptide, or protein corresponds to less than 2-fold increase in mean fluorescence intensity (MFI) shift when compared with a negative control cell line. As used herein, “very high” expression of a ligand, receptor, peptide, or protein corresponds to greater than 20-fold increase in MFI shift when compared with a negative control cell line.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal polypeptide processing (e.g., removal of amino acids from the nascent polypeptide that are not required for biological activity or removal of amino acids by alternative mRNA splicing or alternative polypeptide processing events).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST polypeptide searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

Tables 1-3 list exemplary sequences for the elements and constructs of the system in the presently disclosed subject matter.

TABLE 1
Exemplary amino acid sequences for elements of the system in the presently
disclosed subject matter.

| | | | SEQ |
|---|---|---|---|
| | | | ID |
| Description | | Sequence | NO: |
| Leucine Zippers | RR12EE345L | LEIRAAFLRQRNTALRTEVAELEQEVQRLENEVS | 1 |
| | zipper | QYETRYGPLGGGK | |
| | 2N-two N | LEIEAAFLRQENTALRTEVAELEQEVQRLENEVS | 2 |
| | terminal | QYETRYGPLGGGK | |
| | mutations | | |
| | EE12RR345L | LEIEAAFLERENTALETRVAELRQRVQRLRNRVS | 3 |
| | zipper | QYRTRYGPLGGGK | |
| | 3N-three N | LEIEAAFLRQENTALRTRVAELEQEVQRLENEVS | 4 |
| | terminal | QYETRYGPLGGGK | |
| | mutations | | |
| | 3C-three C | LEIRAAFLRQRNTALRTRVAELEQRVQRLENRVS | 5 |
| | terminal | QYETRYGPLGGGK | |
| | mutations | | |
| | IN-single N | LEIEAAFLRQRNTALRTEVAELEQEVQRLENEVS | 6 |
| | terminal | QYETRYGPLGGGK | |
| | mutation | | |
| | 1M-single | LEIRAAFLRQRNTALRTRVAELEQEVQRLENEVS | 7 |
| | mutation in | QYETRYGPLGGGK | |
| | middle of zipper | | |
| | 2M-two | LEIRAAFLRQRNTALRTRVAELEQRVQRLENEVS | 8 |
| | mutations in | QYETRYGPLGGGK | |
| | middle of zipper | | |
| | synZip-9 zipper | GSQKVESLKQKIEEL | 9 |
| | | KQRKAQLKNDIANLEKEIAYAET | |
| | Fos zipper | GSELTDTLQAETDQLEDEKSALQTEIANLLKEKE | 10 |
| | | KLEFILAAHR | |
| | Jun zipper | ERISRLEEKVKTLKSQNTELASTASLLREQVAQL | 11 |
| | | KQKVLSHVLE | |
| Signal peptides | mouse Ig kappa | METDTLLLWVLLLWVPGSTG | 12 |
| | signal peptide | | |
| | mouse CD8 | MASPLTRFLSLNLLLLGESIILGSGEA | 13 |
| | signal peptide | | |
| | mouse IL-2 | MYSMQLASCVTLTLVLLVNS | 14 |
| | signal peptide | | |
| | human CD8 | MALPVTALLLPLALLLHAARP | 15 |
| | signal peptide | | |
| | mouse Fas | MLWIWAVLPLVLAGSQLRVHT | 16 |
| | signal peptide | | |
| Linkers, Hinge, | Linker1 | GSGRSG | 17 |
| spacers, | Linker2 | GGGGSGGGS | 18 |
| extracellular | Linker3 | GGGGSGGGGSGGGGSTS | 19 |
| domains | Linker4 | GPPVASGSGRSG | 20 |
| | Linker5 | GGGGSGGGSGSL | 21 |
| | Linker6 | GGGGSGGGSELPTQGTFSNVSTNVSGGGGSGGG | 22 |
| | | SRSG | |
| | Linker7 | TSGGGGSGGGSG | 23 |
| | Linker8 | TSGGGGSGGGSGTG | 24 |
| | Linker9 | GGGGSGGGSGTG | 25 |
| | Linker10 | STGGGGSGGGS | 26 |
| | Linker11 | GSGGGGSGGGS | 27 |
| | Linker12 | GTSRAKRGSG | 28 |
| | Linker13 | GGGGSGTG | 29 |
| | Linker14 | GTSRAKRGS | 30 |
| | Linker15 | ALGGSGGGS | 31 |
| | Linker16 | GGGGSTS | 32 |
| | Linker17 | SGGGGSD | 33 |
| | Linker18 | GSSGSG | 34 |
| | Linker19 | GSGGTR | 35 |
| | Linker20 | GSGTR | 36 |
| | Linker21 | TSGSG | 37 |
| | Linker22 | GSGGS | 38 |
| | Linker 23 | GSTGGGGSGGGGSGGGGS | 39 |
| | (interchain | | |
| | linker) | | |
| | Linker 24 | GGGGSGGGGSGGGGSGGGS | 40 |
| | Linker 25 | GGGGSGGGGSGGGGS | 41 |
| | mouse Thy1.1 | QKVTSLTACLVNQNLRLDCRHENNTKDNSIQHE | 42 |
| | spacer and GPI | FSLTREKRKHVLSGTLGIPEHTYRSRVTLSNQPYI | |
| | transfer signal | KVLTLANFTTKDEGDYFCELRVSGANPMSSNKSI | |
| | | SVYRDKLVKCGGISLLVQNTSWMLLLLLSLSLLQ | |
| | | ALDFISL | |
| | Thy1.1 spacer | QKVTSLTACLVDQSLRLDCRHENTSSSPIQYEFSL | 43 |
| | | TRETKKHVLFGTVGVPEHTYRSRTNFTSKYNMK | |
| | | VLYLSAFTSKDEGTYTCALHHSGHSPPISSQNVT | |
| | | VLRDKLVKC | |
| | human EGFRt | RKVCNGIGIGEFKDSLSINATNIKHFKNCTSISGDL | 44 |
| | extracellular | HILPVAFRGDSFTHTPPLDPQELDILKTVKEITGFL | |
| | domain | LIQAWPENRTDLHAFENLEIIRGRTKQHGQFSLA | |
| | | VVSLNITSLGLRSLKEISDGDVIISGNKNLCYANTI | |
| | | NWKKLFGTSGQKTKIISNRGENSCKATGQVCHA | |
| | | LCSPEGCWGPEPRDCVSCRNVSRGRECVDKCNL | |
| | | LEGEPREFVENSECIQCHPECLPQAMNITCTGRGP | |
| | | DNCIQCAHYIDGPHCVKTCPAGVMGENNTLVW | |
| | | KYADAGHVCHLCHPNCTYGCTGPGLEGCPTNGP | |
| | | KIPS | |
| | mouse CD8 | STTTKPVLRTPSPVHPTGTSQPQRPEDCRPRGSVK | 45 |
| | hinge spacer | GTGLDFACD | |
| | mouse CD8 C to | STTTKPVLRTPSPVHPTGTSQPQRPEDSRPRGSVK | 46 |
| | S hinge spacer | GTGLDFASD | |
| | mouse CD28 | IEFMYPPPYLDNERSNGTIIHIKEKHLCHTQSSPKL | 47 |
| | hinge spacer | | |
| | blocking spacer, | CHTQSSPKL | 48 |
| | CD28 | | |
| | blocking spacer, | VPRDCGCKPCICT | 49 |
| | CD28 | | |
| | blocking spacer, | CPSPLFPGPSKP | 50 |
| | CD28 | | |
| | blocking spacer, | EPKSCDKTHTCPPC | 51 |
| | CD28 | | |
| | mouse PD-1 | LEVPNGPWRSLTFYPAWLTVSEGANATFTCSLSN | 52 |
| | extracellular | WSEDLMLNWNRLSPSNQTEKQAAFCNGLSQPV | |
| | domain | QDARFQIIQLPNRHDFHMNILDTRRNDSGIYLCG | |
| | | AISLHPKAKIEESPGAELVVTERILETSTRYPSPSP | |
| | | KPEGRFQGM | |
| | mouse PD-1 | TERILETSTRYPSPSPKPEGRFQGM | 53 |
| | hinge | | |
| | mouse | TDKNQTTQNNSSSPLTQVNTTVSVQIGTKALLCC | 54 |
| | CD200R1 | FSIPLTKAVLITWIIKLRGLPSCTIAYKVDTKTNET | |
| | extracellular | SCLGRNITWASTPDHSPELQISAVTLQHEGTYTCE | |
| | domain | TVTPEGNFEKNYDLQVLVPPEVTYFPEKNRSAVC | |
| | | EAMAGKPAAQISWSPDGDCVTTSESHSNGTVTV | |
| | | RSTCHWEQNNVSDVSCIVSHLTGNQSLSIELSRG | |
| | | GNQSLRP | |
| | mouse Fas | QGTNSISESLKLRRRVRETDKNCSEGLYQGGPFC | 55 |
| | extracellular | CQPCQPGKKKVEDCKMNGGTPTCAPCTEGKEY | |
| | domain | MDKNHYADKCRRCTLCDEEHGLEVETNCTLTQ | |
| | | NTKCKCKPDFYCDSPGCEHCVRCASCEHGTLEP | |
| | | CTATSNTNCRKQSPRNR | |
| Transmembrane | mouse CD28 | FWALVVVAGVLFCYGLLVTVALCVIWT | 56 |
| domains | mouse CD28 | FWALVVVAGVLFCYGLLVTVALCVIWT | 57 |
| | mouse CD8 | IYIWAPLAGICVALLLSLIITLICY | 58 |
| | mouse CD8 | IYIWAPLAGTCGVLLLSLVIT | 59 |
| | mouse CD4 | VFLACVLGGSFGFLGFLGLCILC | 60 |
| | mouse OX40 | AFAVLLGLGLGLLAPLTVLLALYLL | 61 |
| | mouse CD27 | IFVTFSSMFLIFVLGAILFFH | 62 |
| | mouse Fas | LWLLTILVLLIPLVFIY | 63 |
| Cytoplasmic | mouse CD3z | RAKFSRSAETAANLQDTFDALHMQTLAPR | 64 |
| (intracellular) | delta all Y to F | | |
| domains | mouse CD3z | RAKFSRSAETAANLQDTYDALHMQTLAPR | 65 |
| | delta residual Y | | |
| | mouse CD3 zeta | RAKFSRSAETAANLQDPNQLYNELNLGRREEYD | 66 |
| | wild type | VLEKKRARDPEMGGKQQRRRNPQEGVYNALQK | |
| | | DKMAEAYSEIGTKGERRRGKGHDGLYQGLSTAT | |
| | | KDTYDALHMQTLAPR | |
| | mouse CD3 | RAKFSRSAETAANLQDPNQLYNELNLGRREEYD | 67 |
| | 1XX | VLEKKRARDPEMGGKQQRRRNPQEGVFNALQK | |
| | | DKMAEAFSEIGTKGERRRGKGHDGLFQGLSTAT | |
| | | KDTFDALHMQTLAPR | |
| | mouse CD28 | NSRRNRLLQSDYMNMTPRRPGLTRKPYQPYAPA | 68 |
| | | RDFAAYRP | |
| | human 4-1BB | KRGRKKLLYIFKQPFMRPVQTTQEEDGCSCRFPE | 69 |
| | | EEEGGCEL | |
| | mouse OX40 | RKAWRLPNTPKPCWGNSFRTPIQEEHTDAHFTLA | 70 |
| | | KI | |
| | mouse CD27 | QRRNHGPNEDRQAVPEEPCPYSCPREEEGSAIPIQ | 71 |
| | | EDYRKPEPAFYP | |
| Antigen binding | 1D3 CD19 scFv | EVQLQQSGAELVRPGTSVKLSCKVSGDTITFYYM | 72 |
| domains | -VH G4S3 VL | HFVKQRPGQGLEWIGRIDPEDESTKYSEKFKNKA | |
| | | TLTADTSSNTAYLKLSSLTSEDTATYFCIYGGYYF | |
| | | DYWGQGVMVTVSSGGGGSGGGGSGGGGSDIQM | |
| | | TQSPASLSTSLGETVTIQCQASEDIYSGLAWYQQ | |
| | | KPGKSPQLLIYGASDLQDGVPSRFSGSGSGTQYS | |
| | | LKITSMQTEDEGVYFCQQGLTYPRTFGGGTKLEL | |
| | | KR | |
| | 334354 hCD19 | EVQLVESGGGLVQPGGSLRLSCAASGFSFSDFW | 73 |
| | single domain | MSWVRQAPGKGLEWVATISQAGSEKDYVDSVK | |
| | human heavy | GRFTISRDNAKKSLYLQMNSLRAEDTAVYYCAS | |
| | chain VHH- | GVYSFDYRGQGTLVTVSS | |
| | Patent: | | |
| | WO2020018922 | | |
| | A1 | | |
| | 18B12 CD20 | QIVMSQSPAILSASPGEKVTMTCRARSSVSYIHW | 74 |
| | scFv-VL G4S3 | YQQKPGSSPKPWIYATSNLASGVPGRFSGSGSGT | |
| | VH-Patent: | SYSLTITRVEAEDAATYYCQQWSSKPPTFGGGTK | |
| | WO2007064911 | LEIKRGGGGSGGGGSGGGGSQVQLQQPGAELVR | |
| | A1 | PGTSVKLSCKASGYTFTSYWMHWIKQRPGQGLE | |
| | | WIGVIDPSDNYTKYNQKFKGKATLTVDTSSSTAY | |
| | | MQLSSLTSEDSAVYFCAREGYYGSSPWFAYWGQ | |
| | | GTLVTVSS | |
| | 2MC57 CD20 | QVQLQESGGGLVQAGGSLRLSCAASGRDFATYS | 75 |
| | VHH-Patent: | MAWFRQAPGKERESVATISWSGQRTRYADSVK | |
| | US20190092871 | GRFTISRDNAKNTVYLQMNSLKPEDTAVYYCAM | |
| | A1 | PRTWGEFPPTQYDSWGQGTQVTVSS | |
| | HM79-16 | QSILTQPPSISESLGSTARLTCTLRSGISVGNKNIY | 76 |
| | CD79b scFv- | WYQQMAGSAPRLFLYFFSNSDKELGPGVPNRVS | |
| | VL G4S VH | GSKDTSKNAANLQISDLQVEDEAVYYCSIYESNA | |
| | | YVFGSGTQLTVLGGGGSGGGGSGGGGSEVRLLE | |
| | | SGGGLVKPEGSLKLSCVASGFTFSDYFMSWVRQ | |
| | | APGKGLEWVAHIYTKSYNYATYYSGSVKGRFTI | |
| | | SRDDSRRMLYLQMSNLRTEDTATYYCMDGSYG | |
| | | YWGQGTMVTVSS | |
| | CB3s BAFF-R | DIQMTQSPSSLSASVGDRVTITCRASQDVSTAVA | 77 |
| | scFv-VL G4S | WYQQKPGKAPKLLIYSASFLYSGVPSRFSGSGSG | |
| | VH-Patent: | TDFTLTISSLQPEDFATYYCQQSQISPPTFGQGTK | |
| | US20080181888 | VEIKRGGGGSGGGGSGGGGSEVQLVESGGGLVQ | |
| | A1 | PGGSLRLSCAASGFTISSSSIHWVRQAPGKGLEW | |
| | | VAWVLPSVGFTDYADSVKGRFTISADTSKNTAY | |
| | | LQMNSLRAEDTAVYYCARRVCYNRLGVCAGGM | |
| | | DYWGQGTLVTVSS | |
| Affinity tags | Myc | EQKLISEEDL | 78 |
| | human CD34 | ELPTQGTFSNVSTNVSGGGGSGGGSELPTQGTES | 79 |
| | tandem epitope | NVSTNVS | |
| | Q2 | | |
| | Linker in bold, | | |
| | epitopes | | |
| | underlined | | |
| | hCD20 | CPYSNPSLC | 80 |
| | mimotope | | |
| | hCD20 | CPYSNPSLCSGGGGSSGGGGSDCPYSNPSLC | 81 |
| | mimotope “R2” | | |
| | linker in bold, | | |
| | epitopes | | |
| | underlines | | |
| | FLAG | DYKDDDDK | 82 |
| | V5 | GKPIPNPLLGLDST | 83 |
| | twin Strep tag | WSHPQFEKGGGSGGGSGGSAWSHPQFEK | 84 |
| Inhibitory | FasL | MQQPMNYPCPQIFWVDSSATSSWAPPGSVFPCPS | 85 |
| ligands | | CGPRGPDQRRPPPPPPPVSPLPPPSQPLPLPPLTPL | |
| | | KKKDHNTNLWLPVVFFMVLVALVGMGLGMYQ | |
| | | LFHLQKELAELREFTNQSLKVSSFEKQIANPSTPS | |
| | | EKKEPRSVAHLTGNPHSRSIPLEWEDTYGTALISG | |
| | | VKYKKGGLVINETGLYFVYSKVYFRGQSCNNQP | |
| | | LNHKVYMRNSKYPEDLVLMEEKRLNYCTTGQI | |
| | | WAHSSYLGAVENLTSADHLYVNISQLSLINFEES | |
| | | KTFFGLYKL | |
| | PD-L1 | MRIFAGIIFTACCHLLRAFTITAPKDLYVVEYGSN | 86 |
| | | VTMECRFPVERELDLLALVVYWEKEDEQVIQFV | |
| | | AGEEDLKPQHSNFRGRASLPKDQLLKGNAALQIT | |
| | | DVKLQDAGVYCCIISYGGADYKRITLKVNAPYR | |
| | | KINQRISVDPATSEHELICQAEGYPEAEVIWTNSD | |
| | | HQPVSGKRSVTTSRTEGMLLNVTSSLRVNATAN | |
| | | DVFYCTFWRSQPGQNHTAELIIPELPATHPPQNRT | |
| | | HWVLLGSILLFLIVVSTVLLFLRKQVRMLDVEKC | |
| | | GVEDTSSKNRNDTQFEET | |
| | CD200 | MGSLVFRRPFCHLSTYSLIWGMAAVALSTAQVE | 87 |
| | | VVTQDERKALHTTASLRCSLKTSQEPLIVTWQKK | |
| | | KAVSPENMVTYSKTHGVVIQPAYKDRINVTELG | |
| | | LWNSSITFWNTTLEDEGCYMCLFNTFGSQKVSG | |
| | | TACLTLYVQPIVHLHYNYFEDHLNITCSATARPA | |
| | | PAISWKGTGTGIENSTESHFHSNGTTSVTSILRVK | |
| | | DPKTQVGKEVICQVLYLGNVIDYKQSLDKGFWF | |
| | | SVPLLLSIVSLVILLILISILLYWKRHRNQERGESS | |
| | | QGMQRMK | |
| iCaspase9 | iCasp9 | MGVQVETISPGDGRTFPKRGQTCVVHYTGMLED | 88 |
| | | GKKVDSSRDRNKPFKFMLGKQEVIRGWEEGVA | |
| | | QMSVGQRAKLTISPDYAYGATGHPGIIPPHATLV | |
| | | FDVELLKLESGGGSGVDGFGDVGALESLRGNAD | |
| | | LAYILSMEPCGHCLIINNVNFCRESGLRTRTGSNI | |
| | | DCEKLRRRFSSLHFMVEVKGDLTAKKMVLALLE | |
| | | LARQDHGALDCCVVVILSHGCQASHLQFPGAVY | |
| | | GTDGCPVSVEKIVNIFNGTSCPSLGGKPKLFFIQA | |
| | | CGGEQKDHGFEVASTSPEDESPGSNPEPDATPFQ | |
| | | EGLRTFDQLDAISSLPTPSDIFVSYSTFPGFVSWR | |
| | | DPKSGSWYVETLDDIFEQWAHSEDLQSLLLRVA | |
| | | NAVSVKGIYKQMPGCFNFLRKKLFFKTSVD | |
| BCL-2 | mouse BCL-2 | MAQAGRTGYDNREIVMKYIHYKLSQRGYEWDA | 89 |
| | D34A | GAADAAPLGAAPTPGIFSFQPESNPMPAVHRDM | |
| | | AARTSPLRPLVATAGPALSPVPPVVHLTLRRAGD | |
| | | DFSRRYRRDFAEMSSQLHLTPFTARGRFATVVEE | |
| | | LFRDGVNWGRIVAFFEFGGVMCVESVNREMSPL | |
| | | VDNIALWMTEYLNRHLHTWIQDNGGWDAFVEL | |
| | | YGPSMRPLFDFSWLSLKTLLSLALVGACITLGAY | |
| | | LGHK | |
| CD4, CD8 | CD8 | MALPVTALLLPLALLLHAARPSQFRVSPLDRTW | 90 |
| polypeptides | polypeptide | NLGETVELKCQVLLSNPTSGCSWLFQPRGAAASP | |
| | | TFLLYLSQNKPKAAEGLDTQRFSGKRLGDTFVLT | |
| | | LSDFRRENEGYYFCSALSNSIMYFSHFVPVFLPA | |
| | | KPTTTPAPRPPTPAPTIASQPLSLRPEACRPAAGG | |
| | | AVHTRGLDFACDIYIWAPLAGTCGVLLLSLVITL | |
| | | YCNHRNRRRVCKCPRPVVKSGDKPSLSARYV | |
| | CD8 | MASPLTRFLSLNLLLMGESIILGSGEAKPQAPELR | 91 |
| | polypeptide | IFPKKMDAELGQKVDLVCEVLGSVSQGCSWLFQ | |
| | | NSSSKLPQPTFVVYMASSHNKITWDEKLNSSKLF | |
| | | SAVRDTNNKYVLTLNKFSKENEGYYFCSVISNSV | |
| | | MYFSSVVPVLQKVNSTTTKPVLRTPSPVHPTGTS | |
| | | QPQRPEDCRPRGSVKGTGLDFACDIYIWAPLAGI | |
| | | CVAPLLSLIITLICYHRSRKRVCKCPRPLVRQEGK | |
| | | PRPSEKIV | |
| | CD28 | MLRLLLALNLFPSIQVTGNKILVKQSPMLVAYDN | 92 |
| | polypeptide | AVNLSCKYSYNLFSREFRASLHKGLDSAVEVCV | |
| | | VYGNYSQQLQVYSKTGFNCDGKLGNESVTFYLQ | |
| | | NLYVNQTDIYFCKIEVMYPPPYLDNEKSNGTIIHV | |
| | | KGKHLCPSPLFPGPSKPFWVLVVVGGVLACYSL | |
| | | LVTVAFIIFWVRSKRSRLLHSDYMNMTPRRPGPT | |
| | | RKHYQPYAPPRDFAAYRS | |
| | CD4 | MCRAISLRRLLLLLLQLSQLLAVTQGKTLVLGKE | 93 |
| | polypeptide | GESAELPCESSQKKITVFTWKFSDQRKILGQHGK | |
| | | GVLIRGGSPSQFDRFDSKKGAWEKGSFPLIINKLK | |
| | | MEDSQTYICELENRKEEVELWVFKVTFSPGTSLL | |
| | | QGQSLTLTLDSNSKVSNPLTECKHKKGKVVSGS | |
| | | KVLSMSNLRVQDSDFWNCTVTLDQKKNWFGMT | |
| | | LSVLGFQSTAITAYKSEGESAEFSFPLNFAEENGW | |
| | | GELMWKAEKDSFFQPWISFSIKNKEVSVQKSTKD | |
| | | LKLQLKETLPLTLKIPQVSLQFAGSGNLTLTLDK | |
| | | GTLHQEVNLVVMKVAQLNNTLTCEVMGPTSPK | |
| | | MRLTLKQENQEARVSEEQKVVQVVAPETGLWQ | |
| | | CLLSEGDKVKMDSRIQVLSRGVNQTVFLACVLG | |
| | | GSFGFLGFLGLCILCCVRCRHQQRQAARMSQIKR | |
| | | LLSEKKTCQCPHRMQKSHNLI | |
| | CD4 | MNRGVPFRHLLLVLQLALLPAATQGKKVVLGK | 94 |
| | polypeptide | KGDTVELTCTASQKKSIQFHWKNSNQIKILGNQG | |
| | | SFLTKGPSKLNDRADSRRSLWDQGNFPLIIKNLKI | |
| | | EDSDTYICEVEDQKEEVQLLVFGLTANSDTHLLQ | |
| | | GQSLTLTLESPPGSSPSVQCRSPRGKNIQGGKTLS | |
| | | VSQLELQDSGTWTCTVLQNQKKVEFKIDIVVLAF | |
| | | QKASSIVYKKEGEQVEFSFPLAFTVEKLTGSGEL | |
| | | WWQAERASSSKSWITFDLKNKEVSVKRVTQDPK | |
| | | LQMGKKLPLHLTLPQALPQYAGSGNLTLALEAK | |
| | | TGKLHQEVNLVVMRATQLQKNLTCEVWGPTSP | |
| | | KLMLSLKLENKEAKVSKREKAVWVLNPEAGMW | |
| | | QCLLSDSGQVLLESNIKVLPTWSTPVQPMALIVL | |
| | | GGVAGLLLFIGLGIFFCVRCRHRRRQAERMSQIK | |
| | | RLLSEKKTCQCPHRFQKTCSPI | |
| | CD3ζ | MKWKALFTAAILQAQLPITEAQSFGLLDPKLCYL | 95 |
| | polypeptide | LDGILFIYGVILTALFLRVKFSRSADAPAYQQGQN | |
| | | QLYNELNLGRREEYDVLDKRRGRDPEMGGKPQ | |
| | | RRKNPQEGLYNELQKDKMAEAYSEIGMKGERR | |
| | | RGKGHDGLYQGLSTATKDTYDALHMQALPPR | |
| | CD3ζ | MKWKVSVLACILHVRFPGAEAQSFGLLDPKLCY | 96 |
| | polypeptide | LLDGILFIYGVIITALYLRAKFSRSAETAANLQDP | |
| | | NQLYNELNLGRREEYDVLEKKRARDPEMGGKQ | |
| | | RRRNPQEGVYNALQKDKMAEAYSEIGTKGERRR | |
| | | GKGHDGLYQDSHFQAVQFGNRREREGSELTRTL | |
| | | GLRARPKACRHKKPLSLPAAVS | |
| | CD2ζ | MTLRLLFLALNFFSVQVTENKILVKQSPLLVVDS | 97 |
| | polypeptide | NEVSLSCRYSYNLLAKEFRASLYKGVNSDVEVC | |
| | | VGNGNFTYQPQFRSNAEFNCDGDFDNETVTFRL | |
| | | WNLHVNHTDIYFCKIEFMYPPPYLDNERSNGTIIH | |
| | | IKEKHLCHTQSSPKLFWALVVVAGVLFCYGLLV | |
| | | TVALCVIWTNSRRNRLLQSDYMNMTPRRPGLTR | |
| | | KPYQPYAPARDFAAYRP | |
| | 4-1BB | MGNSCYNIVATLLLVLNFERTRSLQDPCSNCPAG | 98 |
| | polypeptide | TFCDNNRNQICSPCPPNSFSSAGGQRTCDICRQCK | |
| | | GVFRTRKECSSTSNAECDCTPGFHCLGAGCSMC | |
| | | EQDCKQGQELTKKGCKDCCFGTFNDQKRGICRP | |
| | | WTNCSLDGKSVLVNGTKERDVVCGPSPADLSPG | |
| | | ASSVTPPAPAREPGHSPQIISFFLALTSTALLFLLFF | |
| | | LTLRFSVVKRGRKKLLYIFKQPFMRPVQTTQEED | |
| | | GCSCRFPEEEEGGCEL | |
| | OX40 | MCVGARRLGRGPCAALLLLGLGLSTVTGLHCVG | 99 |
| | polypeptide | DTYPSNDRCCHECRPGNGMVSRCSRSQNTVCRP | |
| | | CGPGFYNDVVSSKPCKPCTWCNLRSGSERKQLC | |
| | | TATQDTVCRCRAGTQPLDSYKPGVDCAPCPPGH | |
| | | FSPGDNQACKPWTNCTLAGKHTLQPASNSSDAIC | |
| | | EDRDPPATQPQETQGPPARPITVQPTEAWPRTSQ | |
| | | GPSTRPVEVPGGRAVAAILGLGLVLGLLGPLAILL | |
| | | ALYLLRRDQRLPPDAHKPPGGGSFRTPIQEEQAD | |
| | | AHSTLAKI | |
| | ICOS | MKSGLWYFFLFCLRIKVLTGEINGSANYEMFIFH | 100 |
| | polypeptide | NGGVQILCKYPDIVQQFKMQLLKGGQILCDLTKT | |
| | | KGSGNTVSIKSLKFCHSQLSNNSVSFFLYNLDHS | |
| | | HANYYFCNLSIFDPPPFKVTLTGGYLHIYESQLCC | |
| | | QLKFWLPIGCAAFVVVCILGCILICWLTKKKYSSS | |
| | | VHDPNGEYMFMRAVNTAKKSRLTDVTL | |
| 2A peptides | E2A | QCTNYALLKLAGDVESNPGP | 101 |
| | F2A | VKQTLNFDLLKLAGDVESNPGP | 102 |
| | P2A | ATNFSLLKQAGDVEENPGP | 103 |
| | T2A | EGRGSLLTCGDVEENPGP | 104 |

TABLE 2
Exemplary amino acid sequences for constructs of the system
in the presently disclosed subject matter.

| | | SEQ |
|---|---|---|
| | | ID |
| Description | Sequence | NO: |
| RR12EE345L | METDTLLLWVLLLWVPGSTGLEIRAAFLRQRNTALRTEVAELEQE | 105 |
| linker | VQRLENEVSQYETRYGPLGGGKGGGGSGGGGSGGGGSGGGSLEIE | |
| EE12RR345L | AAFLERENTALETRVAELRQRVQRLRNRVSQYRTRYGPLGGGKEF | |
| Thy 1.1 P2A | TQKVTSLTACLVNQNLRLDCRHENNTKDNSIQHEFSLTREKRKHV | |
| CD20 CD8H | LSGTLGIPEHTYRSRVTLSNQPYIKVLTLANFTTKDEGDYFCELR | |
| CD8TM CD28z | VSGANPMSSNKSISVYRDKLVKCGGISLLVQNTSWMLLLLLSLSL | |
| E2A | LQALDFISLGSGATNFSLLKQAGDVEENPGPGSLTMASPLTRFLS | |
| | LNLLLLGESIILGSGEAQIVMSQSPAILSASPGEKVTMTCRARSS | |
| | VSYIHWYQQKPGSSPKPWIYATSNLASGVPGRFSGSGSGTSYSLT | |
| | ITRVEAEDAATYYCQQWSSKPPTFGGGTKLEIKRGGGGSGGGGSG | |
| | GGGSQVQLQQPGAELVRPGTSVKLSCKASGYTFTSYWMHWIKQRP | |
| | GQGLEWIGVIDPSDNYTKYNQKFKGKATLTVDTSSSTAYMQLSSL | |
| | TSEDSAVYFCAREGYYGSSPWFAYWGQGTLVTVSSSAWSHPQFEK | |
| | GGGSGGGSGGSAWSHPQFEKGGGGSGGGSEARLSTTTKPVLRTPS | |
| | PVHPTGTSQPQRPEDCRPRGSVKGTGLDFACDIYIWAPLAGICVA | |
| | LLLSLIITLICYNSRRNRLLQSDYMNMTPRRPGLTRKPYQPYAPA | |
| | RDFAAYRPRAKFSRSAETAANLQDPNQLYNELNLGRREEYDVLEK | |
| | KRARDPEMGGKQQRRRNPQEGVYNALQKDKMAEAYSEIGTKGERR | |
| | RGKGHDGLYQGLSTATKDTYDALHMQTLAPRGSGQCTNYALLKLA | |
| | GDVESNPGPLE | |
| RR12EE345L | METDTLLLWVLLLWVPGSTGLEIRAAFLRQRNTALRTEVAELEQE | 106 |
| linker | VQRLENEVSQYETRYGPLGGGKGGGGSGGGGSGGGGSGGGSLEIE | |
| EE12RR345L | AAFLERENTALETRVAELRQRVQRLRNRVSQYRTRYGPLGGGKEF | |
| Thy 1.1 P2A | TQKVTSLTACLVNQNLRLDCRHENNTKDNSIQHEFSLTREKRKHV | |
| CD20 CD28H | LSGTLGIPEHTYRSRVTLSNQPYIKVLTLANFTTKDEGDYFCELR | |
| CD28TM CD28z | VSGANPMSSNKSISVYRDKLVKCGGISLLVQNTSWMLLLLLSLSL | |
| E2A | LQALDFISLGSGATNFSLLKQAGDVEENPGPGSLTMASPLTRFLS | |
| | LNLLLLGESIILGSGEAQIVMSQSPAILSASPGEKVTMTCRARSS | |
| | VSYIHWYQQKPGSSPKPWIYATSNLASGVPGRFSGSGSGTSYSLT | |
| | ITRVEAEDAATYYCQQWSSKPPTFGGGTKLEIKRGGGGSGGGGSG | |
| | GGGSQVQLQQPGAELVRPGTSVKLSCKASGYTFTSYWMHWIKQRP | |
| | GQGLEWIGVIDPSDNYTKYNQKFKGKATLTVDTSSSTAYMQLSSL | |
| | TSEDSAVYFCAREGYYGSSPWFAYWGQGTLVTVSSSAWSHPQFEK | |
| | GGGSGGGSGGSAWSHPQFEKGGGGSGGGSEARLIEFMYPPPYLDN | |
| | ERSNGTIIHIKEKHLCHTQSSPKLFWALVVVAGVLFCYGLLVTVA | |
| | LCVIWTNSRRNRLLQSDYMNMTPRRPGLTRKPYQPYAPARDFAAY | |
| | RPRAKFSRSAETAANLQDPNQLYNELNLGRREEYDVLEKKRARDP | |
| | EMGGKQQRRRNPQEGVYNALQKDKMAEAYSEIGTKGERRRGKGHD | |
| | GLYQGLSTATKDTYDALHMQTLAPRGSGQCTNYALLKLAGDVESN | |
| | PGPLE | |
| Q2- | METDTLLLWVLLLWVPGSTGGSGELPTQGTFSNVSTNVSGGGGSG | 107 |
| RR12EE345L | GGSELPTQGTFSNVSTNVSGGGGSGGGSALGLEIRAAFLRQRNTA | |
| P2A iC9 F2A | LRTEVAELEQEVQRLENEVSQYETRYGPLGGGKGSGRAKRATNFS | |
| 3xFLAG BCL2 | LLKQAGDVEENPGPEFTRAKRMGVQVETISPGDGRTFPKRGQTCV | |
| D34A E2A CD19 | VHYTGMLEDGKKVDSSRDRNKPFKFMLGKQEVIRGWEEGVAQMSV | |
| CD8H CD8TM | GQRAKLTISPDYAYGATGHPGIIPPHATLVFDVELLKLESGGGSG | |
| CD28z | VDGFGDVGALESLRGNADLAYILSMEPCGHCLIINNVNFCRESGL | |
| | RTRTGSNIDCEKLRRRFSSLHFMVEVKGDLTAKKMVLALLELARQ | |
| | DHGALDCCVVVILSHGCQASHLQFPGAVYGTDGCPVSVEKIVNIF | |
| | NGTSCPSLGGKPKLFFIQACGGEQKDHGFEVASTSPEDESPGSNP | |
| | EPDATPFQEGLRTFDQLDAISSLPTPSDIFVSYSTFPGFVSWRDP | |
| | KSGSWYVETLDDIFEQWAHSEDLQSLLLRVANAVSVKGIYKQMPG | |
| | CFNFLRKKLFFKTSVDGTGRAKRVKQTLNFDLLKLAGDVESNPGP | |
| | GSGDYKDHDGDYKDHDIDYKDDDDKGSGMAQAGRTGYDNREIVMK | |
| | YIHYKLSQRGYEWDAGAADAAPLGAAPTPGIFSFQPESNPMPAVH | |
| | RDMAARTSPLRPLVATAGPALSPVPPVVHLTLRRAGDDFSRRYRR | |
| | DFAEMSSQLHLTPFTARGRFATVVEELFRDGVNWGRIVAFFEFGG | |
| | VMCVESVNREMSPLVDNIALWMTEYLNRHLHTWIQDNGGWDAFVE | |
| | LYGPSMRPLFDFSWLSLKTLLSLALVGACITLGAYLGHKGSGRAK | |
| | RQCTNYALLKLAGDVESNPGPGSLTMASPLTRFLSLNLLLLGESI | |
| | ILGSGEAEVQLQQSGAELVRPGTSVKLSCKVSGDTITFYYMHFVK | |
| | QRPGQGLEWIGRIDPEDESTKYSEKFKNKATLTADTSSNTAYLKL | |
| | SSLTSEDTATYFCIYGGYYFDYWGQGVMVTVSSGGGGSGGGGSGG | |
| | GGSDIQMTQSPASLSTSLGETVTIQCQASEDIYSGLAWYQQKPGK | |
| | SPQLLIYGASDLQDGVPSRFSGSGSGTQYSLKITSMQTEDEGVYF | |
| | CQQGLTYPRTFGGGTKLELKREQKLISEEDLLESSTTTKPVLRTP | |
| | SPVHPTGTSQPQRPEDCRPRGSVKGTGLDFACDIYIWAPLAGICV | |
| | ALLLSLIITLICYNSRRNRLLQSDYMNMTPRRPGLTRKPYQPYAP | |
| | ARDFAAYRPRAKFSRSAETAANLQDPNQLYNELNLGRREEYDVLE | |
| | KKRARDPEMGGKQQRRRNPQEGVYNALQKDKMAEAYSEIGTKGER | |
| | RRGKGHDGLYQGLSTATKDTYDALHMQTLAPRGTEARL | |
| Q2- | METDTLLLWVLLLWVPGSTGGSGELPTQGTFSNVSTNVSGGGGSG | 108 |
| RR12EE345L | GGSELPTQGTFSNVSTNVSGGGGSGGGSALGLEIRAAFLRQRNTA | |
| P2A iC9 F2A | LRTEVAELEQEVQRLENEVSQYETRYGPLGGGKGSGRAKRATNFS | |
| CD19 CD8H | LLKQAGDVEENPGPEFTRAKRMGVQVETISPGDGRTFPKRGQTCV | |
| CD8TM CD28z | VHYTGMLEDGKKVDSSRDRNKPFKFMLGKQEVIRGWEEGVAQMSV | |
| | GQRAKLTISPDYAYGATGHPGIIPPHATLVFDVELLKLESGGGSG | |
| | VDGFGDVGALESLRGNADLAYILSMEPCGHCLIINNVNFCRESGL | |
| | RTRTGSNIDCEKLRRRFSSLHFMVEVKGDLTAKKMVLALLELARQ | |
| | DHGALDCCVVVILSHGCQASHLQFPGAVYGTDGCPVSVEKIVNIF | |
| | NGTSCPSLGGKPKLFFIQACGGEQKDHGFEVASTSPEDESPGSNP | |
| | EPDATPFQEGLRTFDQLDAISSLPTPSDIFVSYSTFPGFVSWRDP | |
| | KSGSWYVETLDDIFEQWAHSEDLQSLLLRVANAVSVKGIYKQMPG | |
| | CFNFLRKKLFFKTSVDGTGRAKRVKQTLNFDLLKLAGDVESNPGP | |
| | GSLTMASPLTRFLSLNLLLLGESIILGSGEAEVQLQQSGAELVRP | |
| | GTSVKLSCKVSGDTITFYYMHFVKQRPGQGLEWIGRIDPEDESTK | |
| | YSEKFKNKATLTADTSSNTAYLKLSSLTSEDTATYFCIYGGYYFD | |
| | YWGQGVMVTVSSGGGGSGGGGSGGGGSDIQMTQSPASLSTSLGET | |
| | VTIQCQASEDIYSGLAWYQQKPGKSPQLLIYGASDLQDGVPSRFS | |
| | GSGSGTQYSLKITSMQTEDEGVYFCQQGLTYPRTFGGGTKLELKR | |
| | EQKLISEEDLLESSTTTKPVLRTPSPVHPTGTSQPQRPEDCRPRG | |
| | SVKGTGLDFACDIYIWAPLAGICVALLLSLIITLICYNSRRNRLL | |
| | QSDYMNMTPRRPGLTRKPYQPYAPARDFAAYRPRAKFSRSAETAA | |
| | NLQDPNQLYNELNLGRREEYDVLEKKRARDPEMGGKQQRRRNPQE | |
| | GVYNALQKDKMAEAYSEIGTKGERRRGKGHDGLYQGLSTATKDTY | |
| | DALHMQTLAPRGTEARL | |
| R2 3N PD-1 | METDTLLLWVLLLWVPGSTGCPYSNPSLCSGGGGSSGGGGSDCPY | 109 |
| DNR P2A CD20 | SNPSLCGGGGSGGGSLEIEAAFLRQENTALRTRVAELEQEVQRLE | |
| CD28H | NEVSQYETRYGPLGGGKGGGGSGGGGSGGGGSGGGSLEIEAAFLE | |
| CD28TM CD28z | RENTALETRVAELRQRVQRLRNRVSQYRTRYGPLGGGKEFTGSTS | |
| E2A Fas-DNR | GSGKPGSGEGSTKGLEVPNGPWRSLTFYPAWLTVSEGANATFTCS | |
| | LSNWSEDLMLNWNRLSPSNQTEKQAAFCNGLSQPVQDARFQIIQL | |
| | PNRHDFHMNILDTRRNDSGIYLCGAISLHPKAKIEESPGAELVVT | |
| | ERILETSTRYPSPSPKPEGRFQGMVFLACVLGGSFGFLGFLGLCI | |
| | LCRAKFSRSAETAANLQDTFDALHMQTLAPRGSGATNFSLLKQAG | |
| | DVEENPGPGSLTMASPLTRFLSLNLLLLGESIILGSGEAQIVMSQ | |
| | SPAILSASPGEKVTMTCRARSSVSYIHWYQQKPGSSPKPWIYATS | |
| | NLASGVPGRFSGSGSGTSYSLTITRVEAEDAATYYCQQWSSKPPT | |
| | FGGGTKLEIKRGGGGSGGGGSGGGGSQVQLQQPGAELVRPGTSVK | |
| | LSCKASGYTFTSYWMHWIKQRPGQGLEWIGVIDPSDNYTKYNQKF | |
| | KGKATLTVDTSSSTAYMQLSSLTSEDSAVYFCAREGYYGSSPWFA | |
| | YWGQGTLVTVSSSAWSHPQFEKGGGSGGGSGGSAWSHPQFEKGGG | |
| | GSGGGSEARLIEFMYPPPYLDNERSNGTIIHIKEKHLCHTQSSPK | |
| | LFWALVVVAGVLFCYGLLVTVALCVIWTNSRRNRLLQSDYMNMTP | |
| | RRPGLTRKPYQPYAPARDFAAYRPRAKFSRSAETAANLQDPNQLY | |
| | NELNLGRREEYDVLEKKRARDPEMGGKQQRRRNPQEGVYNALQKD | |
| | KMAEAYSEIGTKGERRRGKGHDGLYQGLSTATKDTYDALHMQTLA | |
| | PRGSGQCTNYALLKLAGDVESNPGPLEMLWIWAVLPLVLAGSQLR | |
| | VHTQGTNSISESLKLRRRVRETDKNCSEGLYQGGPFCCQPCQPGK | |
| | KKVEDCKMNGGTPTCAPCTEGKEYMDKNHYADKCRRCTLCDEEHG | |
| | LEVETNCTLTQNTKCKCKPDFYCDSPGCEHCVRCASCEHGTLEPC | |
| | TATSNTNCRKQSPRNRLWLLTILVLLIPLVFIYRKYRKRKCWKRR | |
| | QDDPESRTSSRETIPMNASNLSLQKDLGKSTPDTGNENEGQCLE | |
| 3N Thy1.1 P2A | METDTLLLWVLLLWVPGSTGLEIEAAFLRQENTALRTRVAELEQE | 110 |
| CD20 CD28H | VQRLENEVSQYETRYGPLGGGKGGGGSGGGGSGGGGSGGGSLEIE | |
| CD28TM CD28z | AAFLERENTALETRVAELRQRVQRLRNRVSQYRTRYGPLGGGKEF | |
| E2A | TQKVTSLTACLVNQNLRLDCRHENNTKDNSIQHEFSLTREKRKHV | |
| | LSGTLGIPEHTYRSRVTLSNQPYIKVLTLANFTTKDEGDYFCELR | |
| | VSGANPMSSNKSISVYRDKLVKCGGISLLVQNTSWMLLLLLSLSL | |
| | LQALDFISLGSGATNFSLLKQAGDVEENPGPGSLTMASPLTRFLS | |
| | LNLLLLGESIILGSGEAQIVMSQSPAILSASPGEKVTMTCRARSS | |
| | VSYIHWYQQKPGSSPKPWIYATSNLASGVPGRFSGSGSGTSYSLT | |
| | ITRVEAEDAATYYCQQWSSKPPTFGGGTKLEIKRGGGGSGGGGSG | |
| | GGGSQVQLQQPGAELVRPGTSVKLSCKASGYTFTSYWMHWIKQRP | |
| | GQGLEWIGVIDPSDNYTKYNQKFKGKATLTVDTSSSTAYMQLSSL | |
| | TSEDSAVYFCAREGYYGSSPWFAYWGQGTLVTVSSSAWSHPQFEK | |
| | GGGSGGGSGGSAWSHPQFEKGGGGSGGGSEARLIEFMYPPPYLDN | |
| | ERSNGTIIHIKEKHLCHTQSSPKLFWALVVVAGVLFCYGLLVTVA | |
| | LCVIWTNSRRNRLLQSDYMNMTPRRPGLTRKPYQPYAPARDFAAY | |
| | RPRAKFSRSAETAANLQDPNQLYNELNLGRREEYDVLEKKRARDP | |
| | EMGGKQQRRRNPQEGVYNALQKDKMAEAYSEIGTKGERRRGKGHD | |
| | GLYQGLSTATKDTYDALHMQTLAPRGSGQCTNYALLKLAGDVESN | |
| | PGPLE | |
| Q2- | METDTLLLWVLLLWVPGSTGGSGELPTQGTFSNVSTNVSGGGGSG | 111 |
| RR12EE345L | GGSELPTQGTFSNVSTNVSGGGGSGGGSALGLEIRAAFLRQRNTA | |
| P2A iC9 F2A | LRTEVAELEQEVQRLENEVSQYETRYGPLGGGKGSGRAKRATNFS | |
| | LLKQAGDVEENPGPEFTRAKRMGVQVETISPGDGRTFPKRGQTCV | |
| | VHYTGMLEDGKKVDSSRDRNKPFKFMLGKQEVIRGWEEGVAQMSV | |
| | GQRAKLTISPDYAYGATGHPGIIPPHATLVFDVELLKLESGGGSG | |
| | VDGFGDVGALESLRGNADLAYILSMEPCGHCLIINNVNFCRESGL | |
| | RTRTGSNIDCEKLRRRFSSLHFMVEVKGDLTAKKMVLALLELARQ | |
| | DHGALDCCVVVILSHGCQASHLQFPGAVYGTDGCPVSVEKIVNIF | |
| | NGTSCPSLGGKPKLFFIQACGGEQKDHGFEVASTSPEDESPGSNP | |
| | EPDATPFQEGLRTFDQLDAISSLPTPSDIFVSYSTFPGFVSWRDP | |
| | KSGSWYVETLDDIFEQWAHSEDLQSLLLRVANAVSVKGIYKQMPG | |
| | CFNFLRKKLFFKTSVDGTGRAKRVKQTLNFDLLKLAGDVESNPGP | |
| | GSL | |
| 3N PD-1 | METDTLLLWVLLLWVPGSTGLEIEAAFLRQENTALRTRVAELEQE | 112 |
| CD28TM CD28z | VQRLENEVSQYETRYGPLGGGKGGGGSGGGGSGGGGSGGGSLEIE | |
| (PD-1 CAR) | AAFLERENTALETRVAELRQRVQRLRNRVSQYRTRYGPLGGGKEF | |
| | TGSTSGSGKPGSGEGSTKGLEVPNGPWRSLTFYPAWLTVSEGANA | |
| | TFTCSLSNWSEDLMLNWNRLSPSNQTEKQAAFCNGLSQPVQDARF | |
| | QIIQLPNRHDFHMNILDTRRNDSGIYLCGAISLHPKAKIEESPGA | |
| | ELVVTERILETSTRYPSPSPKPEGRFQGMFWALVVVAGVLFCYGL | |
| | LVTVALCVIWTNSRRNRLLQSDYMNMTPRRPGLTRKPYQPYAPAR | |
| | DFAAYRPRAKFSRSAETAANLQDPNQLYNELNLGRREEYDVLEKK | |
| | RARDPEMGGKQQRRRNPQEGVYNALQKDKMAEAYSEIGTKGERRR | |
| | GKGHDGLYQGLSTATKDTYDALHMQTLAPRGSGQCTNYALLKLAG | |
| | DVESNPGPLE | |
| R2 3N PD-1 | METDTLLLWVLLLWVPGSTGCPYSNPSLCSGGGGSSGGGGSDCPY | 113 |
| DNR P2A CD20 | SNPSLCGGGGSGGGSLEIEAAFLRQENTALRTRVAELEQEVQRLE | |
| CD28H | NEVSQYETRYGPLGGGKGGGGSGGGGSGGGGSGGGSLEIEAAFLE | |
| CD28TM CD28z | RENTALETRVAELRQRVQRLRNRVSQYRTRYGPLGGGKEFTGSTS | |
| E2A FasBB | GSGKPGSGEGSTKGLEVPNGPWRSLTFYPAWLTVSEGANATFTCS | |
| | LSNWSEDLMLNWNRLSPSNQTEKQAAFCNGLSQPVQDARFQIIQL | |
| | PNRHDFHMNILDTRRNDSGIYLCGAISLHPKAKIEESPGAELVVT | |
| | ERILETSTRYPSPSPKPEGRFQGMVFLACVLGGSFGFLGFLGLCI | |
| | LCRAKFSRSAETAANLQDTFDALHMQTLAPRGSGATNFSLLKQAG | |
| | DVEENPGPGSLTMASPLTRFLSLNLLLLGESIILGSGEAQIVMSQ | |
| | SPAILSASPGEKVTMTCRARSSVSYIHWYQQKPGSSPKPWIYATS | |
| | NLASGVPGRFSGSGSGTSYSLTITRVEAEDAATYYCQQWSSKPPT | |
| | FGGGTKLEIKRGGGGSGGGGSGGGGSQVQLQQPGAELVRPGTSVK | |
| | LSCKASGYTFTSYWMHWIKQRPGQGLEWIGVIDPSDNYTKYNQKF | |
| | KGKATLTVDTSSSTAYMQLSSLTSEDSAVYFCAREGYYGSSPWFA | |
| | YWGQGTLVTVSSSAWSHPQFEKGGGSGGGSGGSAWSHPQFEKGGG | |
| | GSGGGSEARLIEFMYPPPYLDNERSNGTIIHIKEKHLCHTQSSPK | |
| | LFWALVVVAGVLFCYGLLVTVALCVIWTNSRRNRLLQSDYMNMTP | |
| | RRPGLTRKPYQPYAPARDFAAYRPRAKFSRSAETAANLQDPNQLY | |
| | NELNLGRREEYDVLEKKRARDPEMGGKQQRRRNPQEGVYNALQKD | |
| | KMAEAYSEIGTKGERRRGKGHDGLYQGLSTATKDTYDALHMQTLA | |
| | PRGSGQCTNYALLKLAGDVESNPGPLEMLWIWAVLPLVLAGSQLR | |
| | VHTQGTNSISESLKLRRRVRETDKNCSEGLYQGGPFCCQPCQPGK | |
| | KKVEDCKMNGGTPTCAPCTEGKEYMDKNHYADKCRRCTLCDEEHG | |
| | LEVETNCTLTQNTKCKCKPDFYCDSPGCEHCVRCASCEHGTLEPC | |
| | TATSNTNCRKQSPRNRLWLLTILVLLIPLVFIYKRGRKKLLYIFK | |
| | QPFMRPVQTTQEEDGCSCRFPEEEEGGCEL | |
| 3N Thy1.1 P2A | METDTLLLWVLLLWVPGSTGLEIEAAFLRQENTALRTRVAELEQE | 114 |
| CD20 CD28H | VQRLENEVSQYETRYGPLGGGKGGGGSGGGGSGGGGSGGGSLEIE | |
| CD28TM CD28z | AAFLERENTALETRVAELRQRVQRLRNRVSQYRTRYGPLGGGKEF | |
| E2A FasBB | TQKVTSLTACLVNQNLRLDCRHENNTKDNSIQHEFSLTREKRKHV | |
| | LSGTLGIPEHTYRSRVTLSNQPYIKVLTLANFTTKDEGDYFCELR | |
| | VSGANPMSSNKSISVYRDKLVKCGGISLLVQNTSWMLLLLLSLSL | |
| | LQALDFISLGSGATNFSLLKQAGDVEENPGPGSLTMASPLTRFLS | |
| | LNLLLLGESIILGSGEAQIVMSQSPAILSASPGEKVTMTCRARSS | |
| | VSYIHWYQQKPGSSPKPWIYATSNLASGVPGRFSGSGSGTSYSLT | |
| | ITRVEAEDAATYYCQQWSSKPPTFGGGTKLEIKRGGGGSGGGGSG | |
| | GGGSQVQLQQPGAELVRPGTSVKLSCKASGYTFTSYWMHWIKQRP | |
| | GQGLEWIGVIDPSDNYTKYNQKFKGKATLTVDTSSSTAYMQLSSL | |
| | TSEDSAVYFCAREGYYGSSPWFAYWGQGTLVTVSSSAWSHPQFEK | |
| | GGGSGGGSGGSAWSHPQFEKGGGGSGGGSEARLIEFMYPPPYLDN | |
| | ERSNGTIIHIKEKHLCHTQSSPKLFWALVVVAGVLFCYGLLVTVA | |
| | LCVIWTNSRRNRLLQSDYMNMTPRRPGLTRKPYQPYAPARDFAAY | |
| | RPRAKFSRSAETAANLQDPNQLYNELNLGRREEYDVLEKKRARDP | |
| | EMGGKQQRRRNPQEGVYNALQKDKMAEAYSEIGTKGERRRGKGHD | |
| | GLYQGLSTATKDTYDALHMQTLAPRGSGQCTNYALLKLAGDVESN | |
| | PGPLEMLWIWAVLPLVLAGSQLRVHTQGTNSISESLKLRRRVRET | |
| | DKNCSEGLYQGGPFCCQPCQPGKKKVEDCKMNGGTPTCAPCTEGK | |
| | EYMDKNHYADKCRRCTLCDEEHGLEVETNCTLTQNTKCKCKPDFY | |
| | CDSPGCEHCVRCASCEHGTLEPCTATSNTNCRKQSPRNRLWLLTI | |
| | LVLLIPLVFIYKRGRKKLLYIFKQPFMRPVQTTQEEDGCSCRFPE | |
| | EEEGGCEL | |
| Q2- | METDTLLLWVLLLWVPGSTGGSGELPTQGTFSNVSTNVSGGGGSG | 115 |
| RR12EE345L | GGSELPTQGTFSNVSTNVSGGGGSGGGSALGLEIRAAFLRQRNTA | |
| P2A CD8SP R2 | LRTEVAELEQEVQRLENEVSQYETRYGPLGGGKGSGRAKRATNFS | |
| PD-1H delta F2A | LLKQAGDVEENPGPEFTMALPVTALLLPLALLLHAARPCPYSNPS | |
| CD200R1 CD27 | LCSGGGGSSGGGGSDCPYSNPSLCGGGGSGGGSTERILETSTRYP | |
| E2A CD19 | SPSPKPEGRFQGMFWALVVVAGVLFCYGLLVTVALCVIWTRAKFS | |
| CD8H CD8TM | RSAETAANLQDTFDALHMQTLAPRGTGRAKRVKQTLNFDLLKLAG | |
| CD28z | DVESNPGPGSGMYSMQLASCVTLTLVLLVNSTDKNQTTQNNSSSP | |
| | LTQVNTTVSVQIGTKALLCCFSIPLTKAVLITWIIKLRGLPSCTI | |
| | AYKVDTKTNETSCLGRNITWASTPDHSPELQISAVTLQHEGTYTC | |
| | ETVTPEGNFEKNYDLQVLVPPEVTYFPEKNRSAVCEAMAGKPAAQ | |
| | ISWSPDGDCVTTSESHSNGTVTVRSTCHWEQNNVSDVSCIVSHLT | |
| | GNQSLSIELSRGGNQSLRPIFVTFSSMFLIFVLGAILFFHQRRNH | |
| | GPNEDRQAVPEEPCPYSCPREEEGSAIPIQEDYRKPEPAFYPGSG | |
| | QCTNYALLKLAGDVESNPGPGSLTMASPLTRFLSLNLLLLGESII | |
| | LGSGEAEVQLQQSGAELVRPGTSVKLSCKVSGDTITFYYMHFVKQ | |
| | RPGQGLEWIGRIDPEDESTKYSEKFKNKATLTADTSSNTAYLKLS | |
| | SLTSEDTATYFCIYGGYYFDYWGQGVMVTVSSGGGGSGGGGSGGG | |
| | GSDIQMTQSPASLSTSLGETVTIQCQASEDIYSGLAWYQQKPGKS | |
| | PQLLIYGASDLQDGVPSRFSGSGSGTQYSLKITSMQTEDEGVYFC | |
| | QQGLTYPRTFGGGTKLELKREQKLISEEDLLESSTTTKPVLRTPS | |
| | PVHPTGTSQPQRPEDCRPRGSVKGTGLDFACDIYIWAPLAGICVA | |
| | LLLSLIITLICYNSRRNRLLQSDYMNMTPRRPGLTRKPYQPYAPA | |
| | RDFAAYRPRAKFSRSAETAANLQDPNQLYNELNLGRREEYDVLEK | |
| | KRARDPEMGGKQQRRRNPQEGVYNALQKDKMAEAYSEIGTKGERR | |
| | RGKGHDGLYQGLSTATKDTYDALHMQTLAPRGTEARL | |
| R2 3N PD-1 | METDTLLLWVLLLWVPGSTGCPYSNPSLCSGGGGSSGGGGSDCPY | 116 |
| OX40 P2A CD20 | SNPSLCGGGGSGGGSLEIEAAFLRQENTALRTRVAELEQEVQRLE | |
| CD28H | NEVSQYETRYGPLGGGKGGGGSGGGGSGGGGSGGGSLEIEAAFLE | |
| CD28TM CD28z | RENTALETRVAELRQRVQRLRNRVSQYRTRYGPLGGGKEFTGSTS | |
| E2A FasBB | GSGKPGSGEGSTKGTSLEVPNGPWRSLTFYPAWLTVSEGANATFT | |
| | CSLSNWSEDLMLNWNRLSPSNQTEKQAAFCNGLSQPVQDARFQII | |
| | QLPNRHDFHMNILDTRRNDSGIYLCGAISLHPKAKIEESPGAELV | |
| | VTERILETSTRYPSPSPKPEGRFQGMAFAVLLGLGLGLLAPLTVL | |
| | LALYLLRKAWRLPNTPKPCWGNSFRTPIQEEHTDAHFTLAKIGSG | |
| | ATNFSLLKQAGDVEENPGPGSLTMASPLTRFLSLNLLLLGESIIL | |
| | GSGEAQIVMSQSPAILSASPGEKVTMTCRARSSVSYIHWYQQKPG | |
| | SSPKPWIYATSNLASGVPGRFSGSGSGTSYSLTITRVEAEDAATY | |
| | YCQQWSSKPPTFGGGTKLEIKRGGGGSGGGGSGGGGSQVQLQQPG | |
| | AELVRPGTSVKLSCKASGYTFTSYWMHWIKQRPGQGLEWIGVIDP | |
| | SDNYTKYNQKFKGKATLTVDTSSSTAYMQLSSLTSEDSAVYFCAR | |
| | EGYYGSSPWFAYWGQGTLVTVSSSAWSHPQFEKGGGSGGGSGGSA | |
| | WSHPQFEKGGGGSGGGSEARLIEFMYPPPYLDNERSNGTIIHIKE | |
| | KHLCHTQSSPKLFWALVVVAGVLFCYGLLVTVALCVIWTNSRRNR | |
| | LLQSDYMNMTPRRPGLTRKPYQPYAPARDFAAYRPRAKFSRSAET | |
| | AANLQDPNQLYNELNLGRREEYDVLEKKRARDPEMGGKQQRRRNP | |
| | QEGVYNALQKDKMAEAYSEIGTKGERRRGKGHDGLYQGLSTATKD | |
| | TYDALHMQTLAPRGSGQCTNYALLKLAGDVESNPGPLEMLWIWAV | |
| | LPLVLAGSQLRVHTQGTNSISESLKLRRRVRETDKNCSEGLYQGG | |
| | PFCCQPCQPGKKKVEDCKMNGGTPTCAPCTEGKEYMDKNHYADKC | |
| | RRCTLCDEEHGLEVETNCTLTQNTKCKCKPDFYCDSPGCEHCVRC | |
| | ASCEHGTLEPCTATSNTNCRKQSPRNRLWLLTILVLLIPLVFIYK | |
| | RGRKKLLYIFKQPFMRPVQTTQEEDGCSCRFPEEEEGGCEL | |
| Q2- | METDTLLLWVLLLWVPGSTGGSGELPTQGTFSNVSTNVSGGGGSG | 117 |
| RR12EE345L | GGSELPTQGTFSNVSTNVSGGGGSGGGSALGLEIRAAFLRQRNTA | |
| P2A R2 BAFF-R | LRTEVAELEQEVQRLENEVSQYETRYGPLGGGKGSGRAKRATNFS | |
| R2 CD28H | LLKQAGDVEENPGPGSLTMASPLTRFLSLNLLLLGESIILGSGEA | |
| CD28TM CD28 | CPYSNPSLCSGGGGSSGGGGSDCPYSNPSLCGGGGSGGGSDIQMT | |
| 1XX E2A CD79b | QSPSSLSASVGDRVTITCRASQDVSTAVAWYQQKPGKAPKLLIYS | |
| FLAG PD-1H | ASFLYSGVPSRFSGSGSGTDFTLTISSLQPEDFATYYCQQSQISP | |
| CD8TM CD28 | PTFGQGTKVEIKRGGGGSGGGGSGGGGSEVQLVESGGGLVQPGGS | |
| 1XX | LRLSCAASGFTISSSSIHWVRQAPGKGLEWVAWVLPSVGFTDYAD | |
| | SVKGRFTISADTSKNTAYLQMNSLRAEDTAVYYCARRVCYNRLGV | |
| | CAGGMDYWGQGTLVTVSSSGGGGSDCPYSNPSLCSGGGGSDCPYS | |
| | NPSLCGGGGSGGGSEARLIEFMYPPPYLDNERSNGTIIHIKEKHL | |
| | CHTQSSPKLFWALVVVAGVLFCYGLLVTVALCVIWTNSRRNRLLQ | |
| | SDYMNMTPRRPGLTRKPYQPYAPARDFAAYRPRAKFSRSAETAAN | |
| | LQDPNQLYNELNLGRREEYDVLEKKRARDPEMGGKQQRRRNPQEG | |
| | VFNALQKDKMAEAFSEIGTKGERRRGKGHDGLFQGLSTATKDTFD | |
| | ALHMQTLAPRGSGQCTNYALLKLAGDVESNPGPLEGMYSMQLASC | |
| | VTLTLVLLVNSQSILTQPPSISESLGSTARLTCTLRSGISVGNKN | |
| | IYWYQQMAGSAPRLFLYFFSNSDKELGPGVPNRVSGSKDTSKNAA | |
| | NLQISDLQVEDEAVYYCSIYESNAYVFGSGTQLTVLGGGGSGGGG | |
| | SGGGGSEVRLLESGGGLVKPEGSLKLSCVASGFTFSDYFMSWVRQ | |
| | APGKGLEWVAHIYTKSYNYATYYSGSVKGRFTISRDDSRRMLYLQ | |
| | MSNLRTEDTATYYCMDGSYGYWGQGTMVTVSSAAADYKDDDDKTE | |
| | RILETSTRYPSPSPKPEGRFQGMALGIYIWAPLAGICVALLLSLI | |
| | ITLICYNSRRNRLLQSDYMNMTPRRPGLTRKPYQPYAPARDFAAY | |
| | RPRAKFSRSAETAANLQDPNQLYNELNLGRREEYDVLEKKRARDP | |
| | EMGGKQQRRRNPQEGVFNALQKDKMAEAFSEIGTKGERRRGKGHD | |
| | GLFQGLSTATKDTFDALHMQTLAPR | |
| 3N Thy1.1 P2A | METDTLLLWVLLLWVPGSTGLEIEAAFLRQENTALRTRVAELEQE | 118 |
| CD20 CD8H | VQRLENEVSQYETRYGPLGGGKGGGGSGGGGSGGGGSGGGSLEIE | |
| CD8TM CD28 | AAFLERENTALETRVAELRQRVQRLRNRVSQYRTRYGPLGGGKEF | |
| 1XX | TQKVTSLTACLVNQNLRLDCRHENNTKDNSIQHEFSLTREKRKHV | |
| | LSGTLGIPEHTYRSRVTLSNQPYIKVLTLANFTTKDEGDYFCELR | |
| | VSGANPMSSNKSISVYRDKLVKCGGISLLVQNTSWMLLLLLSLSL | |
| | LQALDFISLGSGATNFSLLKQAGDVEENPGPGSLTMASPLTRFLS | |
| | LNLLLLGESIILGSGEAQIVMSQSPAILSASPGEKVTMTCRARSS | |
| | VSYIHWYQQKPGSSPKPWIYATSNLASGVPGRFSGSGSGTSYSLT | |
| | ITRVEAEDAATYYCQQWSSKPPTFGGGTKLEIKRGGGGSGGGGSG | |
| | GGGSQVQLQQPGAELVRPGTSVKLSCKASGYTFTSYWMHWIKQRP | |
| | GQGLEWIGVIDPSDNYTKYNQKFKGKATLTVDTSSSTAYMQLSSL | |
| | TSEDSAVYFCAREGYYGSSPWFAYWGQGTLVTVSSSAWSHPQFEK | |
| | GGGSGGGSGGSAWSHPQFEKGGGGSGGGSEARLSTTTKPVLRTPS | |
| | PVHPTGTSQPQRPEDCRPRGSVKGTGLDFACDIYIWAPLAGICVA | |
| | LLLSLIITLICYNSRRNRLLQSDYMNMTPRRPGLTRKPYQPYAPA | |
| | RDFAAYRPRAKFSRSAETAANLQDPNQLYNELNLGRREEYDVLEK | |
| | KRARDPEMGGKQQRRRNPQEGVFNALQKDKMAEAFSEIGTKGERR | |
| | RGKGHDGLFQGLSTATKDTFDALHMQTLAPRGSGQCTNYALLKLA | |
| | GDVESNPGPLE | |
| R2 3N PD-1 | METDTLLLWVLLLWVPGSTGCPYSNPSLCSGGGGSSGGGGSDCPY | 119 |
| OX40 P2A CD20 | SNPSLCGGGGSGGGSLEIEAAFLRQENTALRTRVAELEQEVQRLE | |
| CD8H CD8TM | NEVSQYETRYGPLGGGKGGGGSGGGGSGGGGSGGGSLEIEAAFLE | |
| CD28 1XX E2A | RENTALETRVAELRQRVQRLRNRVSQYRTRYGPLGGGKEFTGSTS | |
| FasBB | GSGKPGSGEGSTKGTSLEVPNGPWRSLTFYPAWLTVSEGANATFT | |
| | CSLSNWSEDLMLNWNRLSPSNQTEKQAAFCNGLSQPVQDARFQII | |
| | QLPNRHDFHMNILDTRRNDSGIYLCGAISLHPKAKIEESPGAELV | |
| | VTERILETSTRYPSPSPKPEGRFQGMAFAVLLGLGLGLLAPLTVL | |
| | LALYLLRKAWRLPNTPKPCWGNSFRTPIQEEHTDAHFTLAKIGSG | |
| | ATNFSLLKQAGDVEENPGPGSLTMASPLTRFLSLNLLLLGESIIL | |
| | GSGEAQIVMSQSPAILSASPGEKVTMTCRARSSVSYIHWYQQKPG | |
| | SSPKPWIYATSNLASGVPGRFSGSGSGTSYSLTITRVEAEDAATY | |
| | YCQQWSSKPPTFGGGTKLEIKRGGGGSGGGGSGGGGSQVQLQQPG | |
| | AELVRPGTSVKLSCKASGYTFTSYWMHWIKQRPGQGLEWIGVIDP | |
| | SDNYTKYNQKFKGKATLTVDTSSSTAYMQLSSLTSEDSAVYFCAR | |
| | EGYYGSSPWFAYWGQGTLVTVSSSAWSHPQFEKGGGSGGGSGGSA | |
| | WSHPQFEKGGGGSGGGSEARLSTTTKPVLRTPSPVHPTGTSQPQR | |
| | PEDCRPRGSVKGTGLDFACDIYIWAPLAGICVALLLSLIITLICY | |
| | NSRRNRLLQSDYMNMTPRRPGLTRKPYQPYAPARDFAAYRPRAKF | |
| | SRSAETAANLQDPNQLYNELNLGRREEYDVLEKKRARDPEMGGKQ | |
| | QRRRNPQEGVENALQKDKMAEAFSEIGTKGERRRGKGHDGLFQGL | |
| | STATKDTFDALHMQTLAPRGSGQCTNYALLKLAGDVESNPGPLEM | |
| | LWIWAVLPLVLAGSQLRVHTQGTNSISESLKLRRRVRETDKNCSE | |
| | GLYQGGPFCCQPCQPGKKKVEDCKMNGGTPTCAPCTEGKEYMDKN | |
| | HYADKCRRCTLCDEEHGLEVETNCTLTQNTKCKCKPDFYCDSPGC | |
| | EHCVRCASCEHGTLEPCTATSNTNCRKQSPRNRLWLLTILVLLIP | |
| | LVFIYKRGRKKLLYIFKQPFMRPVQTTQEEDGCSCRFPEEEEGGC | |
| | EL | |
| 334354 hCD19 | MYSMQLASCVTLTLVLLVNSEVQLVESGGGLVQPGGSLRLSCAAS | 120 |
| VHH myc CD8H | GFSFSDFWMSWVRQAPGKGLEWVATISQAGSEKDYVDSVKGRFTI | |
| CS CD8TM | SRDNAKKSLYLQMNSLRAEDTAVYYCASGVYSFDYRGQGTLVTVS | |
| CD28 1XX E2A | SEQKLISEEDLAAASTTTKPVLRTPSPVHPTGTSQPQRPEDSRPR | |
| 2MC57 VHH | GSVKGTGLDFASDALGIYIWAPLAGICVALLLSLIITLICYNSRR | |
| CD20 ST CD8H | NRLLQSDYMNMTPRRPGLTRKPYQPYAPARDFAAYRPRAKFSRSA | |
| CD8TM CD28 | ETAANLQDPNQLYNELNLGRREEYDVLEKKRARDPEMGGKQQRRR | |
| 1XX P2A V5 3N | NPQEGVFNALQKDKMAEAFSEIGTKGERRRGKGHDGLFQGLSTAT | |
| PD-1H delta | KDTFDALHMQTLAPRGSGQCTNYALLKLAGDVESNPGPLEGMASP | |
| | LTRFLSLNLLLLGESIILGSGEAQVQLQESGGGLVQAGGSLRLSC | |
| | AASGRDFATYSMAWFRQAPGKERESVATISWSGQRTRYADSVKGR | |
| | FTISRDNAKNTVYLQMNSLKPEDTAVYYCAMPRTWGEFPPTQYDS | |
| | WGQGTQVTVSSSAWSHPQFEKGGGSGGGSGGSAWSHPQFEKGGGG | |
| | SGGGSEARLSTTTKPVLRTPSPVHPTGTSQPQRPEDCRPRGSVKG | |
| | TGLDFACDIYIWAPLAGICVALLLSLIITLICYNSRRNRLLQSDY | |
| | MNMTPRRPGLTRKPYQPYAPARDFAAYRPRAKFSRSAETAANLQD | |
| | PNQLYNELNLGRREEYDVLEKKRARDPEMGGKQQRRRNPQEGVFN | |
| | ALQKDKMAEAFSEIGTKGERRRGKGHDGLFQGLSTATKDTFDALH | |
| | MQTLAPRGSGATNFSLLKQAGDVEENPGPTRSMETDTLLLWVLLL | |
| | WVPGSTGGSGGKPIPNPLLGLDSTGGGGSGGGSLEIEAAFLRQEN | |
| | TALRTRVAELEQEVQRLENEVSQYETRYGPLGGGKGGGGSGGGGS | |
| | GGGGSGGGSLEIEAAFLERENTALETRVAELRQRVQRLRNRVSQY | |
| | RTRYGPLGGGKGGGGSGGGSTERILETSTRYPSPSPKPEGRFQGM | |
| | FWALVVVAGVLFCYGLLVTVALCVIWTRAKFSRSAETAANLQDTF | |
| | DALHMQTLAPR | |
| 334354 hCD19 | MYSMQLASCVTLTLVLLVNSEVQLVESGGGLVQPGGSLRLSCAAS | 121 |
| VHH myc CD8H | GFSFSDFWMSWVRQAPGKGLEWVATISQAGSEKDYVDSVKGRFTI | |
| CS CD8TM | SRDNAKKSLYLQMNSLRAEDTAVYYCASGVYSFDYRGQGTLVTVS | |
| CD28 1XX E2A | SEQKLISEEDLAAASTTTKPVLRTPSPVHPTGTSQPQRPEDSRPR | |
| 2MC57 VHH | GSVKGTGLDFASDALGIYIWAPLAGICVALLLSLIITLICYNSRR | |
| CD20 ST CD8H | NRLLQSDYMNMTPRRPGLTRKPYQPYAPARDFAAYRPRAKFSRSA | |
| CD8TM CD28 | ETAANLQDPNQLYNELNLGRREEYDVLEKKRARDPEMGGKQQRRR | |
| 1XX P2A R2 3N | NPQEGVFNALQKDKMAEAFSEIGTKGERRRGKGHDGLFQGLSTAT | |
| CD200R1 CD27 | KDTFDALHMQTLAPRGSGQCTNYALLKLAGDVESNPGPLEGMASP | |
| | LTRFLSLNLLLLGESIILGSGEAQVQLQESGGGLVQAGGSLRLSC | |
| | AASGRDFATYSMAWFRQAPGKERESVATISWSGQRTRYADSVKGR | |
| | FTISRDNAKNTVYLQMNSLKPEDTAVYYCAMPRTWGEFPPTQYDS | |
| | WGQGTQVTVSSSAWSHPQFEKGGGSGGGSGGSAWSHPQFEKGGGG | |
| | SGGGSEARLSTTTKPVLRTPSPVHPTGTSQPQRPEDCRPRGSVKG | |
| | TGLDFACDIYIWAPLAGICVALLLSLIITLICYNSRRNRLLQSDY | |
| | MNMTPRRPGLTRKPYQPYAPARDFAAYRPRAKFSRSAETAANLQD | |
| | PNQLYNELNLGRREEYDVLEKKRARDPEMGGKQQRRRNPQEGVFN | |
| | ALQKDKMAEAFSEIGTKGERRRGKGHDGLFQGLSTATKDTFDALH | |
| | MQTLAPRGSGATNFSLLKQAGDVEENPGPTRSMETDTLLLWVLLL | |
| | WVPGSTGCPYSNPSLCSGGGGSSGGGGSDCPYSNPSLCGGGGSGG | |
| | GSLEIEAAFLRQENTALRTRVAELEQEVQRLENEVSQYETRYGPL | |
| | GGGKGGGGSGGGGSGGGGSGGGSLEIEAAFLERENTALETRVAEL | |
| | RQRVQRLRNRVSQYRTRYGPLGGGKEFTGSTSGSGKPGSGEGSTK | |
| | GTSTDKNQTTQNNSSSPLTQVNTTVSVQIGTKALLCCFSIPLTKA | |
| | VLITWIIKLRGLPSCTIAYKVDTKTNETSCLGRNITWASTPDHSP | |
| | ELQISAVTLQHEGTYTCETVTPEGNFEKNYDLQVLVPPEVTYFPE | |
| | KNRSAVCEAMAGKPAAQISWSPDGDCVTTSESHSNGTVTVRSTCH | |
| | WEQNNVSDVSCIVSHLTGNQSLSIELSRGGNQSLRPIFVTFSSMF | |
| | LIFVLGAILFFHQRRNHGPNEDRQAVPEEPCPYSCPREEEGSAIP | |
| | IQEDYRKPEPAFYP | |
| IL-2SP PD-1 | MYSMQLASCVTLTLVLLVNSTSLEVPNGPWRSLTFYPAWLTVSEG | 122 |
| OX40 P2A | ANATFTCSLSNWSEDLMLNWNRLSPSNQTEKQAAFCNGLSQPVQD | |
| FasBB | ARFQIIQLPNRHDFHMNILDTRRNDSGIYLCGAISLHPKAKIEES | |
| | PGAELVVTERILETSTRYPSPSPKPEGRFQGMAFAVLLGLGLGLL | |
| | APLTVLLALYLLRKAWRLPNTPKPCWGNSFRTPIQEEHTDAHFTL | |
| | AKIGSGATNFSLLKQAGDVEENPGPGSLTRAKRGSGLEMLWIWAV | |
| | LPLVLAGSQLRVHTQGTNSISESLKLRRRVRETDKNCSEGLYQGG | |
| | PFCCQPCQPGKKKVEDCKMNGGTPTCAPCTEGKEYMDKNHYADKC | |
| | RRCTLCDEEHGLEVETNCTLTQNTKCKCKPDFYCDSPGCEHCVRC | |
| | ASCEHGTLEPCTATSNTNCRKQSPRNRLWLLTILVLLIPLVFIYK | |
| | RGRKKLLYIFKQPFMRPVQTTQEEDGCSCRFPEEEEGGCEL | |

TABLE 3
Exemplary nucleic acid sequences of the system
in the presently disclosed subject matter.

| | | SEQ |
|---|---|---|
| | | ID |
| Description | Sequence | NO: |
| CD38 gRNA | UAAAUUCAUAGUUAGCCAUU | 123 |
| CD19 gRNA | UGAUUCAAACUGCUCCCCCG | 124 |
| CD22 gRNA | UGUCAUUGGCACGUAUCGGG | 125 |
| RR12EE345L zipper | CTGGAGATCCGCGCTGCATTTTTGCGACAAAGAAATACC | 126 |
| | GCTTTGCGAACCGAAGTGGCCGAGTTGGAGCAGGAAGTA | |
| | CAACGCCTGGAAAATGAAGTCAGCCAATACGAAACTCGA | |
| | TATGGACCTCTCGGAGGTGGAAAG | |
| RR12EE345L zipper | CTGGAGATCGAAGCTGCATTTTTGCGACAAAGAAATACC | 127 |
| | GCTTTGCGAACCGAAGTGGCCGAGTTGGAGCAGGAAGTA | |
| | CAACGCCTGGAAAATGAAGTCAGCCAATACGAAACTCGA | |
| | TATGGACCTCTCGGAGGTGGAAAG | |
| RR12EE345L zipper | CTGGAGATCCGCGCTGCATTTTTGCGACAAAGAAATACC | 128 |
| | GCTTTGCGAACCCGCGTGGCCGAGTTGGAGCAGGAAGTA | |
| | CAACGCCTGGAAAATGAAGTCAGCCAATACGAAACTCGA | |
| | TATGGACCTCTCGGAGGTGGAAAG | |
| RR12EE345L zipper | CTGGAGATCGAAGCTGCATTTTTGCGACAAGAAAATACC | 129 |
| | GCTTTGCGAACCGAAGTGGCCGAGTTGGAGCAGGAAGTA | |
| | CAACGCCTGGAAAATGAAGTCAGCCAATACGAAACTCGA | |
| | TATGGACCTCTCGGAGGTGGAAAG | |
| RR12EE345L zipper | CTGGAGATCCGCGCTGCATTTTTGCGACAAAGAAATACC | 130 |
| | GCTTTGCGAACCCGCGTGGCCGAGTTGGAGCAGCGCGTA | |
| | CAACGCCTGGAAAATGAAGTCAGCCAATACGAAACTCGA | |
| | TATGGACCTCTCGGAGGTGGAAAG | |
| RR12EE345L zipper | CTGGAGATCGAAGCTGCATTTTTGCGACAAGAAAATACC | 131 |
| | GCTTTGCGAACCCGAGTGGCCGAGTTGGAGCAGGAAGTA | |
| | CAACGCCTGGAAAATGAAGTCAGCCAATACGAAACTCGA | |
| | TATGGACCTCTCGGAGGTGGAAAG | |
| RR12EE345L zipper | CTGGAGATCCGCGCTGCATTTTTGCGACAAAGAAATACC | 132 |
| | GCTTTGCGAACCCGAGTGGCCGAGTTGGAGCAGCGAGTA | |
| | CAACGCCTGGAAAATCGAGTCAGCCAATACGAAACTCGA | |
| | TATGGACCTCTCGGAGGTGGAAAG | |
| RR12EE345L zipper | CTGGAGATTGAGGCTGCATTTCTTGAAAGAGAAAACACT | 133 |
| | GCTCTTGAGACAAGAGTCGCCGAACTGAGGCAGCGCGTT | |
| | CAGCGCCTGCGGAACCGAGTATCTCAATACAGGACTCGG | |
| | TACGGACCACTGGGGGGCGGTAAG | |

### 2. Membrane-Bound Leucine Zipper Polypeptides and Soluble Leucine Zipper Polypeptides

The presently disclosed subject matter provides, in certain embodiments, leucine zipper-based sorting systems comprising a plurality of nucleic acid constructs, wherein the plurality comprises: a first nucleic acid construct encoding a membrane bound polypeptide (capture zipper) comprising: (i) an extracellular domain comprising a first leucine zipper sequence; (ii) a transmembrane domain; and (iii) an intracellular domain; and a second nucleic acid construct encoding a soluble polypeptide (secreted zipper) comprising a second leucine zipper sequence capable of heterodimerizing with the first leucine zipper sequence and a signal peptide sequence. The membrane bound polypeptide comprises an extracellular domain comprising a first leucine zipper sequence.

### 2.1 Membrane-Bound Polypeptide

In certain embodiments, the membrane-bound polypeptide comprises a transmembrane domain and an extracellular domain. In certain embodiments, the membrane-bound polypeptide further comprises a hinge/spacer domain and/or an intracellular domain.

### 2.1.1. Extracellular Domain

In certain embodiments, the extracellular domain comprises a dimerization domain that is capable of dimerizing with one or more dimerization domain comprised in the membrane-bound polypeptide. In certain embodiments, the dimerization domain is capable of dimerizing with one or more dimerization domains comprised in a soluble polypeptide disclosed herein. In certain embodiments, the extracellular domain of the membrane-bound polypeptide comprises a first dimerization domain and a second dimerization domain that is capable of dimerizing with the first dimerization domain at a cell surface. In certain embodiments, the extracellular domain comprises a dimerization domain comprising a first leucine zipper sequence (capture zipper) that is capable of heterodimerizing with one or more dimerization domains comprised in the soluble polypeptide disclosed herein. In certain embodiments, the extracellular domain comprises a dimerization domain comprising a first leucine zipper sequence (capture zipper) that is capable of heterodimerizing with a second leucine zipper sequence (secretory zipper) comprised in the soluble polypeptide disclosed herein.

In certain embodiments, the leucine zipper domain comprises a dimerization domain of the Basic-region leucine zipper (bZIP) class of eukaryotic transcription factors. In certain embodiments, the leucine zipper domain comprises a specific alpha helix monomer that can dimerize with anther alpha helix monomer. In certain embodiments, the leucine zipper domain comprises an EE domain that comprises one or more acidic amino acids, e.g., glutamic acid (E). In certain embodiments, the leucine zipper domain comprises an RR domain that comprises one or more basic amino acids, e.g., arginine (R). In certain embodiments, the first leucine zipper domain comprises an RR domain and the second leucine zipper domain comprises an EE domain.

In certain embodiments, the RR domain comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 1 or a fragment thereof. In certain embodiments, the RR domain comprises a modification of SEQ ID NO: 1 or a fragment thereof. In certain embodiments, the modification comprises up to one, up to two, or up to three amino acid substitutions.

An exemplary nucleotide sequence encoding the amino acid sequence of SEQ ID NO: 1 is set forth in SEQ ID NO: 126.

In certain embodiments, the RR domain comprises a modification of SEQ ID NO: 1, wherein the modification consists of or has one amino acid substitution. In certain embodiments, the RR domain comprises the amino acid sequence set forth in SEQ ID NO: 5 or SEQ ID NO: 6.

An exemplary nucleotide sequence encoding the amino acid sequence of SEQ ID NO: 5 is set forth in SEQ ID NO: 127.

An exemplary nucleotide sequence encoding the amino acid sequence of SEQ ID NO: 6 is set forth in SEQ ID NO: 128. In certain embodiments, the RR domain comprises a modification of SEQ ID NO: 1, wherein the modification consists of or has two amino acid substitutions. In certain embodiments, the RR domain comprises the amino acid sequence set forth in SEQ ID NO: 2 or SEQ ID NO: 7.

An exemplary nucleotide sequence encoding the amino acid sequence of SEQ ID NO: 63 is set forth in SEQ ID NO: 129.

An exemplary nucleotide sequence encoding the amino acid sequence of SEQ ID NO: 7 is set forth in SEQ ID NO: 130.

In certain embodiments, the RR domain comprises a modification of SEQ ID NO: 1, wherein the modification consists of or has three amino acid substitutions. In certain embodiments, the RR domain comprises the amino acid sequence set forth in SEQ ID NO: 3 or SEQ ID NO: 4.

An exemplary nucleotide sequence encoding the amino acid sequence of SEQ ID NO: 3 is set forth in SEQ ID NO: 131.

An exemplary nucleotide sequence encoding the amino acid sequence of SEQ ID NO: 4 is set forth in SEQ ID NO: 132.

In certain embodiment, the modification is positioned in the “g” residues of the RR domain of the leucine zipper. In certain embodiment, the modification reduces heterodimerization affinity between the membrane-bound polypeptide and a linked soluble polypeptide.

In certain embodiments, the EE domain comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 3 or a fragment thereof. In certain embodiments, the EE domain comprises a modification of SEQ ID NO: 3 or a fragment thereof. In certain embodiments, the modification comprises up to one, up to two, or up to three amino acid substitutions.

An exemplary nucleotide sequence encoding the amino acid sequence of SEQ ID NO: 2 is set forth in SEQ ID NO: 133.

In certain embodiments, the extracellular domain further comprises a linker between the first dimerization domain and the second dimerization domain. In certain embodiments, the linker comprises the amino acid sequence set forth in any one of SEQ ID NOs: 14-38.

In certain embodiments, a dimerization domain comprises an orthogonal zipper. Orthogonal zippers are coiled coil domains that form heterodimers with their specific partner only and not with other zipper domains. In certain embodiments, orthogonality refers to sets of molecules (e.g., leucine zippers) that are non-cross-reactive, i.e., “orthogonal”, to other sets of molecules. For example, A+B=AB and C+D=CD, but neither A nor B bind to C or D, and vice versa.

In certain embodiments, the first and second leucine zipper sequences of the membrane-bound polypeptide are a pair of orthogonal zippers i.e., the first and the second leucine zipper domains are the specific partners for each other to form heterodimers. Orthogonal zippers include, but are not limited to, RR/EE zippers, Fos/Jun zippers and Fos/synZip zippers. Fos/Jun zippers are previously disclosed in Ransone et al., Genes Dev. 1989 June; 3(6):770-81; Kohler et al., Biochemistry. (2001 January); 9; 40(1):130-42, which are incorporated by reference herein. Fos/synZip zippers are previously disclosed in Grigoryan et al., Nature. (2009); 458, 859-864; Reinke et al., J Am Chem Soc. (2010); 132, 6025-6031, which are incorporated by reference herein.

In certain embodiments, the orthogonal zippers comprise an amino acid sequence that is at least about 80%, at least about 85%, at least about 90%, at least about 95%, at least about 96%, at least about 97%, at least about 98%, at least about 99% or at least about 100% homologous or identical to RR/EE zippers, Fos/Jun zippers or Fos/synZip zippers, or a fragment thereof, and/or may optionally comprise up to one or up to two or up to three conservative amino acid substitutions.

Examples of synZip-9, Fos and Jun zippers are set forth in SEQ ID NOs: 9, 10 and 11, respectively.

In certain embodiments, the extracellular domain of the membrane-bound polypeptide further comprises a spacer/hinge domain between a dimerization domain and a transmembrane domain.

In certain embodiments, the spacer/hinge domain is flexible enough to allow the dimerization domain to orient in different directions to facilitate antigen recognition after dimerizing with the soluble polypeptide disclosed herein. The spacer region can be the hinge region from IgG1, or the CH₂CH₃ region of immunoglobulin and fragments of CD3, a fragment of a CD28 polypeptide, a fragment of a CD8 polypeptide, a variation of any of the foregoing that is at least about 80%, at least about 85%, at least about 90%, at least about 95%, at least about 96%, at least about 97%, at least about 98%, at least about 99% or at least about 100% identical thereto, or a synthetic spacer sequence.

In certain embodiments, the spacer/hinge domain comprises an epitope recognized by an antibody. In certain embodiments, binding of the antibody to the epitope mediates a deletion of a cell comprising the membrane-bound polypeptide. In certain embodiments, the spacer/hinge domain comprises a Thy1.1 molecule, a truncated EGFR molecule (EGFRt), CD22 immunoglobulin-like domain epitope, an IgG/Fc domain (can be a Fc from any IgG), CD2, CD20 cyclic mimotope, CD30, CD52, or HER2.

In certain embodiments, the Thy1.1 molecule comprises or has the amino acid sequence set forth in SEQ ID NO: 42. In certain embodiments, the Thy1.1 molecule comprises or has the amino acid sequence set forth in SEQ ID NO: 43.

In certain embodiments, the EGFRt comprises or has the amino acid sequence set forth in SEQ ID NO: 44.

In certain embodiments, the membrane-bound polypeptide further comprises a blocking spacer, wherein the blocking spacer is capable of preventing dimerization of the membrane-bound polypeptide with a soluble polypeptide when the membrane-bound polypeptide and the soluble polypeptide are not expressed from the same cell. In certain embodiments, the blocking spacer comprises a minimum spacer of no more than about 20 to about 30 amino acid residues. In certain embodiments, the blocking spacer comprises no more than about 25 amino acid residues. In certain embodiments, the blocking spacer comprises about 1, about 2, about 3, about 4, about 5, about 6, about 7, about 8, about 9 about 10, about 11, about 12, about 13, about 14, about 15, about 16, about 17, about 18, about 19 or about 20 amino acid residues. In certain embodiments, the blocking spacer comprises between about 5 amino acid residues and about 25 amino acid residues, between about 5 amino acid residues and about 20 amino acid residues, between about 10 amino acid residues and about 25 amino acid residues or between about 10 amino acid residues and about 20 amino acid residues.

In certain embodiments, the blocking spacer comprises an amino acid sequence that is at least about 80%, at least about 85%, at least about 90%, at least about 95%, at least about 96%, at least about 97%, at least about 98%, at least about 99% or at least about 100% homologous or identical to a truncated CD28 spacer set forth in SEQ ID NO: 48 or 50, or a fragment thereof. In certain embodiments, the blocking spacer comprises an amino acid sequence that is at least about 80%, at least about 85%, at least about 90%, at least about 95%, at least about 96%, at least about 97%, at least about 98%, at least about 99% or at least about 100% homologous to an IgG1 hinge set forth in SEQ ID NO: 49 or SEQ ID NO: 51, or a fragment thereof. In certain embodiments, the blocking spacer comprises a modification of SEQ ID NO: 48, SEQ ID NO: 49, SEQ ID NO: 50, and SEQ ID NO: 51, wherein the modification comprises up to one, up to two, or up to three amino acid substitutions.

In certain embodiments, the blocking spacer has a length of no more than about 25 amino acids. In certain embodiments, the blocking spacer has a length of between about 5 amino acids and about 25 amino acids. In certain embodiments, the blocking spacer is a truncated CD28 spacer or an IgG1 hinge.

In certain non-limiting embodiments, the extracellular domain of the membrane-bound polypeptide comprises a sequence of at least one co-stimulatory ligand or a fragment thereof. Non-limiting examples of co-stimulatory ligands include tumor necrosis factor (TNF) family members, immunoglobulin (Ig) superfamily members, and a combination thereof. In certain embodiments, the co-stimulatory ligand is selected from the group consisting of tumor necrosis factor (TNF) family members. In certain embodiments, the TNF family member is selected from the group consisting of OX40, CD40, CD30, 4-1BB, CD27, RANK, GITR, LTBR, HVEM, BAFF-R, TACI, BCMA, TROY, CD70, GITRL, CD40L, and CD30L, fragments thereof, or combinations thereof.

In certain embodiments, the intracellular domain comprises a member of the immunoglobulin superfamily. In a non-limiting embodiment, the intracellular domain comprises an intracellular domain of CD28.

4. In certain non-limiting embodiments, the extracellular domain comprises an extracellular domain of an immune checkpoint molecule. Non-limiting examples of immune checkpoint molecule include PD-1, Fas, CD200R1, TIGIT, ICOS, CTLA-4, BTLA, TIM-3, LAG-3, leukocyte associated immunoglobulin like receptor (LAIR1), herpesvirus entry mediator (HVEM), 2B4 (CD244), CD160, Galectin9, VISTA, PSGL-1, or fragments thereof or combinations thereof.

In certain embodiments, the immune checkpoint molecule is a dominant negative molecule, e.g., a dominant negative receptor (DNR) or variant thereof. In certain embodiments, the dominant negative molecule is selected from the group consisting of inhibitors of immune checkpoint molecules, tumor necrosis factor receptor superfamily (TNFRSF) members, and TGFP receptors. Non-limiting examples of DNRs include PD-1-DNR, Fas-DNR, CD200R1-DNR, TIGIT-DNR, ICOS-DNR, CTLA-4-DNR, BTLA-DNR, TIM-3-DNR, LAG-3-DNR, LAIR1-DNR, HVEM-DNR, CD244-DNR, CD160-DNR, VISTA-DNR, PSGL-1-DNR, variants thereof, or combinations thereof. Details of dominant negative (DN) forms of inhibitors of an immune checkpoint molecule are disclosed in WO2017/040945 and WO2017/100428, the contents of each of which are incorporated by reference herein in their entireties. In certain embodiments, the extracellular domain of the membrane-bound polypeptide further comprises a dominant negative form of an immune checkpoint inhibitor disclosed in WO2017/040945. In certain embodiments, the extracellular domain of the membrane-bound polypeptide further comprises a dominant negative form of an immune checkpoint inhibitor disclosed in WO2017/100428.

In some embodiments, the immune checkpoint molecule is PD-1 having an extracellular domain set forth in SEQ ID NO: 52. In some embodiments, the immune checkpoint molecule is CD200R1 having an extracellular domain set forth in SEQ ID NO: 54. In some embodiments, the immune checkpoint molecule is Fas having an extracellular domain set forth in SEQ ID NO: 55.

In certain embodiments, the dominant negative molecule is a PD-1 dominant negative (i.e., PD-1 DNR) molecule. In certain embodiments, the PD-1 DN comprises (a) at least a fragment of an extracellular domain of PD-1 comprising a ligand binding region, and (b) a transmembrane domain.

In certain embodiments, the PD-1 DN is a mouse PD-1 DN. In certain embodiments, the PD-1 DN comprises or has the amino acid sequence set forth in SEQ ID NO: 52.

In certain embodiments, the dominant negative molecule is a CD200R1 dominant negative receptor (i.e., CD200R1-DNR) molecule. In certain embodiments, the CD200R1-DNR comprises (a) at least a fragment of an extracellular domain of CD200R1 comprising a ligand binding region, and (b) a transmembrane domain.

In certain embodiments, the CD200R1-DNR is a mouse CD200R1-DNR. In certain embodiments, the CD200R1-DNR comprises or has the amino acid sequence set forth in SEQ ID NO: 54.

In certain embodiments, the dominant negative molecule is a Fas dominant negative (i.e., Fas-DNR) molecule. In certain embodiments, the Fas-DNR comprises (a) at least a fragment of an extracellular domain of Fas comprising a ligand binding region, and (b) a transmembrane domain.

In certain embodiments, the Fas-DNR is a mouse Fas-DNR. In certain embodiments, the Fas-DNR comprises or has the amino acid sequence set forth in SEQ ID NO: 55.

In certain embodiments, the extracellular domain of the membrane-bound polypeptide further comprises a tag. In certain embodiments, the tag comprises an epitope tag recognized by a first antibody. Non-limiting examples of epitope tags include Myc-tag, a HA-tag, a Flag-tag, a V5-tag, and a T7-tag.

In certain embodiments, the tag comprises an affinity tag that binds to a substrate. Non-limiting examples of affinity tags include a His-tag, a Strep-tag, an E-tag, and a streptavidin binding protein tag (SBP-tag).

Furthermore, the extracellular domain of the membrane-bound polypeptide can further comprise a mimotope recognized by a second antibody. Binding of the second antibody to the mimotope can mediates depletion of a cell comprising the membrane-bound polypeptide. In certain embodiments, the mimotope is a CD20 mimotope recognized by an anti-CD20 antibody. In certain embodiments, the anti-CD20 antibody is Rituximab. In certain embodiments, the CD20 mimotope is a circular CD20 mimotope.

In certain embodiments, the CD20 mimotope comprises or has the amino acid sequence set forth in SEQ ID NO: 80.

### 2.1.2. Transmembrane Domain

In accordance with the presently disclosed subject matter, the transmembrane domain can comprise a CD8 polypeptide (e.g., the transmembrane domain of CD8 or a fragment thereof), a CD28 polypeptide (e.g., the transmembrane domain of CD28 or a fragment thereof), a CD3ζ polypeptide (e.g., the transmembrane domain of CD3ζ or a fragment thereof), a CD4 polypeptide (e.g., the transmembrane domain of CD4 or a fragment thereof), a 4-1BB polypeptide (e.g., the transmembrane domain of 4-1BB or a fragment thereof), an OX40 polypeptide (e.g., the transmembrane domain of OX40 or a fragment thereof), an ICOS polypeptide (e.g., the transmembrane domain of ICOS or a fragment thereof), a CD2 polypeptide (e.g., the transmembrane domain of CD2 or a fragment thereof), a synthetic peptide (not based on a protein associated with the immune response), or a combination thereof.

In certain embodiments, the transmembrane domain of the membrane-bound polypeptide comprises a CD8 polypeptide (e.g., the transmembrane domain of CD8 or a fragment thereof). In certain embodiments, the CD8 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001139345.1 (SEQ ID NO: 90) or a fragment thereof, and/or may optionally comprise up to one or up to two or up to three conservative amino acid substitutions. In certain embodiments, the CD8 polypeptide comprises or has an amino acid sequence that is a consecutive fragment of SEQ ID NO: 90, which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 150 to 200, 183 to 203, or 200 to 235 of SEQ ID NO: 90. In certain embodiments, the transmembrane domain of the membrane-bound polypeptide comprises a CD8 polypeptide comprising or having an amino acid sequence of amino acids 183 to 203 of SEQ ID NO: 90.

In certain embodiments, the CD8 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: AAA92533.1 (SEQ ID NO: 91) or a fragment thereof, and/or may optionally comprise up to one or up to two or up to three conservative amino acid substitutions. In certain embodiments, the CD8 polypeptide comprises or has an amino acid sequence that is a consecutive fragment of SEQ ID NO: 91 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, or 200 to 247 of SEQ ID NO: 91.

In certain embodiments, the CD8 polypeptide comprises or has the amino acid sequence set forth in SEQ ID NO: 58.

In certain embodiments, the CD8 polypeptide comprises or has the amino acid sequence set forth in SEQ ID NO: 59.

In certain embodiments, the transmembrane domain of the membrane-bound polypeptide comprises a CD28 polypeptide (e.g., the transmembrane domain of CD28 or a fragment thereof). The CD28 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P10747 or NP_006130 (SEQ ID No: 92), or a fragment thereof, and/or may optionally comprise up to one or up to two or up to three conservative amino acid substitutions. In certain embodiments, the CD28 polypeptide comprises or has an amino acid sequence that is a consecutive fragment of SEQ ID NO: 92 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, 153 to 179, or 200 to 220 of SEQ ID NO: 92. In certain embodiments, the transmembrane domain of a presently disclosed membrane-bound polypeptide comprises a CD28 polypeptide comprising or having an amino acid sequence of amino acids 153 to 179 of SEQ ID NO: 92.

In certain embodiments, the transmembrane domain of a membrane-bound polypeptide comprises a CD28 polypeptide comprising or having the amino acid sequence set forth in SEQ ID NO: 56.

In certain embodiments, the transmembrane domain of a membrane-bound polypeptide comprises a CD28 polypeptide comprising or having the amino acid sequence set forth in SEQ ID NO: 57.

In certain embodiments, the transmembrane domain of the membrane-bound polypeptide comprises a CD4 polypeptide (e.g., the transmembrane domain of CD4 or a fragment thereof). The CD4 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_038516.1 (SEQ ID NO: 93) or a fragment thereof, and/or may optionally comprise up to one or up to two or up to three conservative amino acid substitutions. In certain embodiments, the CD4 polypeptide comprises or has an amino acid sequence that is a consecutive fragment of SEQ ID NO: 93 which is at least 20, or at least 30, or at least 40, or at least 50, and up to 457 amino acids in length. Alternatively or additionally, in non-limiting various embodiments, the CD4 polypeptide comprises or has an amino acid sequence of amino acids 1 to 457, 1 to 50, 50 to 100, 100 to 150, 150 to 200, 200 to 250, 250 to 300, 300 to 350, 350 to 400, 395 to 417, or 400 to 457 of SEQ ID NO: 93. In certain embodiments, the transmembrane domain of the membrane-bound polypeptide comprises a CD4 polypeptide comprising or having amino acids 395 to 417 of SEQ ID NO: 93. In certain embodiments, the transmembrane domain of the membrane-bound polypeptide comprises a CD4 polypeptide (e.g., the transmembrane domain of CD4 or a fragment thereof). The CD4 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_000607.1 (SEQ ID No: 94) or a fragment thereof, and/or may optionally comprise up to one or up to two or up to three conservative amino acid substitutions. In certain embodiments, the CD4 polypeptide comprises or has an amino acid sequence that is a consecutive fragment of SEQ ID NO: 94 which is at least 20, or at least 30, or at least 40, or at least 50, and up to 458 amino acids in length. Alternatively or additionally, in non-limiting various embodiments, the CD4 polypeptide comprises or has an amino acid sequence of amino acids 1 to 457, 1 to 50, 50 to 100, 100 to 150, 150 to 200, 200 to 250, 250 to 300, 300 to 350, 350 to 400, 397 to 418, or 400 to 457 of SEQ ID NO: 94. In certain embodiments, the transmembrane domain of the membrane-bound polypeptide comprises a CD4 polypeptide comprising or having amino acids 397 to 418 of SEQ ID NO: 94.

### 2.1.3. Intracellular Domain

In certain non-limiting embodiments, the membrane-bound polypeptide further comprises an intracellular domain. In certain non-limiting embodiments, the intracellular domain provides an activation signal to a cell (e.g., a cell of the lymphoid lineage, e.g., a T cell). In certain embodiments, the intracellular domain of the membrane-bound polypeptides comprises an immune activating molecule. In certain embodiments, the immune activating molecule is a CD3ζ polypeptide.

In certain non-limiting embodiments, the intracellular domain of the membrane-bound polypeptide comprises a CD3ζ polypeptide or a fragment thereof. CD3ζ can activate or stimulate a cell. CD3ζ comprises 3 ITAMs and transmits an activation signal to the cell (e.g., a cell of the lymphoid lineage, e.g., a T cell) after antigen is bound. The intracellular signaling domain of the CD3ζ-chain is the primary transmitter of signals from endogenous TCRs. In certain embodiments, the CD3ζ polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_932170 (SEQ ID No: 95), or a fragment thereof, and/or may optionally comprise up to one or up to two or up to three conservative amino acid substitutions. In certain non-limiting embodiments, the CD3ζ polypeptide comprises or has an amino acid sequence that is a consecutive fragment of SEQ ID NO: 95, which is at least 20, or at least 30, or at least 40, or at least 50, and up to 164 amino acids in length. Alternatively or additionally, in non-limiting various embodiments, the CD3ζ polypeptide comprises or has an amino acid sequence of amino acids 1 to 164, 1 to 50, 50 to 100, 52 to 164, 100 to 950, or 950 to 164 of SEQ ID NO: 95. In certain embodiments, the CD3ζ polypeptide comprises or has an amino acid sequence of amino acids 52 to 164 of SEQ ID NO: 95.

In certain embodiments, the CD3ζ polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001106864.2 (SEQ ID No: 96) or a fragment thereof, and/or may optionally comprise up to one or up to two or up to three conservative amino acid substitutions. In certain non-limiting embodiments, the CD3ζ polypeptide comprises or has an amino acid sequence that is a consecutive fragment of SEQ ID NO: 96, which is at least about 20, or at least about 30, or at least about 40, or at least about 50, or at least about 90, or at least about 100, and up to 188 amino acids in length. Alternatively or additionally, in non-limiting various embodiments, the CD3ζ polypeptide comprises or has an amino acid sequence of amino acids 1 to 164, 1 to 50, 50 to 100, 100 to 150, or 150 to 188 of SEQ ID NO: 96.

In certain embodiments, the CD3ζ polypeptide comprises or has an amino acid sequence set forth in SEQ ID NO: 66.

In certain embodiments, the intracellular domain of the membrane-bound polypeptide comprises a murine CD3ζ polypeptide.

In certain embodiments, the intracellular domain of the membrane-bound polypeptide comprises a human CD3ζ polypeptide.

In certain non-limiting embodiments, the intracellular domain of the membrane-bound polypeptide provides an activation signal and a stimulation signal to a cell. In certain embodiments, the intracellular of the membrane-bound polypeptide domain comprises at least one costimulatory molecule or a fragment thereof.

In certain embodiments, the at least one co-stimulatory signaling region comprises a CD28 polypeptide (e.g., the intracellular domain of CD28 or a fragment thereof), a 4-1BB polypeptide (e.g., the intracellular domain of 4-1BB or a fragment thereof), an OX40 polypeptide (e.g., the intracellular domain of OX40 or a fragment thereof), an ICOS polypeptide (e.g., the intracellular domain of ICOS or a fragment thereof), a DAP-10 polypeptide (e.g., the intracellular domain of DAP-10 or a fragment thereof), or a combination thereof. In certain embodiments, the at least one co-stimulatory signaling region comprises a CD28 polypeptide. In certain embodiments, the at least one co-stimulatory signaling region comprises an intracellular domain of CD28 or a fragment thereof.

In certain embodiments, the costimulatory molecule is a CD28 polypeptide (e.g., the intracellular domain of CD28 or a fragment thereof). The CD28 polypeptide can comprise or have an amino acid sequence that is at least about 80%, at least about 85%, at least about 90%, at least about 95%, at least about 96%, at least about 97%, at least about 98%, at least about 99% or at least 100% homologous or identical to the sequence having a NCBI Reference No: P10747 or NP_006130 (SEQ ID NO: 14) or a fragment thereof, and/or may optionally comprise up to one or up to two or up to three conservative amino acid substitutions. In certain embodiments, the CD28 polypeptide comprises or has an amino acid sequence that is a consecutive fragment of SEQ ID NO: 92 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 150 to 200, or 200 to 220 of SEQ ID NO: 92. In certain embodiments, the CD28 polypeptide comprises or has an amino acid sequence of amino acids 181 to 220 of SEQ ID NO: 92.

In certain embodiments, the CD28 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_031668.3 (SEQ ID NO: 16) or a fragment thereof, and/or may optionally comprise up to one or up to two or up to three conservative amino acid substitutions. In certain embodiments, the CD28 polypeptide comprises or has an amino acid sequence that is a consecutive fragment of SEQ ID NO: 97 which is at least about 20, or at least about 30, or at least about 40, or at least about 50, and up to 218 amino acids in length. Alternatively or additionally, in non-limiting various embodiments, the CD28 polypeptide comprises or has an amino acid sequence of amino acids 1 to 218, 1 to 50, 50 to 100, 100 to 150, 150 to 200, or 200 to 218 of SEQ ID NO: 97. In certain embodiments, the CD28 polypeptide comprises or has an amino acid sequence of amino acids 178 to 218 of SEQ ID NO: 97.

In certain embodiments, the costimulatory molecule is a mouse CD28 polypeptide. In certain embodiments, the costimulatory molecule is a human CD28 polypeptide.

In certain embodiments, the intracellular domain of the membrane-bound polypeptide comprises two costimulatory molecules, e.g., CD28 and 4-1BB or CD28 and OX40.

In certain embodiments, the at least one co-stimulatory signaling region comprises a 4-1BB polypeptide. In certain embodiments, the at least one co-stimulatory signaling region comprises an intracellular domain of 4-1BB or a fragment thereof.

In certain embodiments, the costimulatory molecule is a 4-1BB polypeptide (e.g., the intracellular domain of 4-1BB or a fragment thereof). 4-1BB can act as a tumor necrosis factor (TNF) ligand and have stimulatory activity. The 4-1BB polypeptide can comprise or have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P41273 or NP_001552 (SEQ ID NO: 98) or a fragment thereof, and/or may optionally comprise up to one or up to two or up to three conservative amino acid substitutions.

In accordance with the presently disclosed subject matter, a “4-1BB nucleic acid molecule” refers to a polynucleotide encoding a 4-1BB polypeptide.

In certain embodiments, the intracellular domain comprises a member of the TNFRSF. In certain embodiments, the intracellular domain comprises an intracellular domain of one or more co-stimulatory signaling domains. Non-limiting examples of co-stimulatory signaling domains include intracellular domain of OX40, CD40, CD30, 4-1BB, CD27, RANK, GITR, LTBR, HVEM, BAFF-R, TACI, BCMA, TROY, fragments thereof, or combinations thereof.

In certain embodiments, the intracellular domain comprises a member of the immunoglobulin superfamily. In a non-limiting embodiment, the intracellular domain comprises an intracellular domain of CD28.

In certain embodiments, the system encodes a switch receptor wherein the extracellular domain comprises an inhibitor molecule (e.g. immune checkpoint molecule) or fragment thereof that is attached via a transmembrane domain to an intracellular stimulatory molecule, whereby binding of an inhibitory ligand to the inhibitory molecule on the cell surface switches the signaling into a stimulatory signal inside the cell. Non-limiting examples of such switch receptors include PD-1-OX40, Fas-4-1BB (FasBB), CD200R1-CD27, TIGIT-4-1BB (TIGITBB), ICOS-CD27, variants thereof, or combinations thereof.

In certain embodiments, the costimulatory molecule is an OX40 polypeptide (e.g., the intracellular domain of OX40 or a fragment thereof). The OX40 polypeptide can comprise or have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P43489 or NP_003318 (SEQ ID NO: 99) or a fragment thereof, and/or may optionally comprise up to one or up to two or up to three conservative amino acid substitutions.

In accordance with the presently disclosed subject matter, an “OX40 nucleic acid molecule” refers to a polynucleotide encoding an OX40 polypeptide.

In certain embodiments, the at least one co-stimulatory signaling region comprises an ICOS polypeptide. In certain embodiments, the at least one co-stimulatory signaling region comprises an intracellular domain of ICOS or a fragment thereof.

In certain embodiments, the costimulatory molecule is an ICOS polypeptide (e.g., the intracellular domain of ICOS or a fragment thereof). The ICOS polypeptide can comprise or have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_036224 (SEQ ID NO: 100) or a fragment thereof, and/or may optionally comprise up to one or up to two or up to three conservative amino acid substitutions.

In accordance with the presently disclosed subject matter, an “ICOS nucleic acid molecule” refers to a polynucleotide encoding an ICOS polypeptide.

In certain embodiments, the at least one co-stimulatory signaling region comprises two costimulatory molecules or fragments thereof. In certain embodiments, the at least one co-stimulatory signaling region comprises a CD28 polypeptide (e.g., an intracellular domain of CD28 or a fragment thereof) and a 4-1BB polypeptide (e.g., an intracellular domain of 4-1BB or a fragment thereof).

In certain non-limiting embodiments, the intracellular domain of the membrane-bound polypeptide alone does not provide an activation signal to a cell. In certain embodiments, the intracellular domain of the membrane-bound polypeptide does not comprise a costimulatory molecule. In certain embodiments, the intracellular domain of the membrane-bound polypeptide does not comprise a CD3zeta polypeptide.

In certain embodiments, the intracellular domain of the membrane-bound polypeptide comprises a CD3 domain, a costimulatory domain, a suicide gene product, survival gene product, fragments thereof, or combinations thereof.

In certain embodiments, the intracellular domain of the membrane-bound polypeptide comprises a suicide gene. Suitable suicide genes include, but are not limited to, Herpes simplex virus thymidine kinase (hsv-tk), and inducible Caspase 9 Suicide gene (iCaspase9 or iCasp9).

In certain embodiments, the iCasp9 comprises a sequence as set forth in SEQ ID NO: 88

In certain embodiments, the intracellular domain of the membrane-bound polypeptide comprises a survival gene. In certain embodiments, the survival gene product is a caspase resistant Bcl-2.

In certain embodiments, the caspase resistant Bcl-2 comprises a sequence as set forth in SEQ ID NO: 89

In certain embodiments, the intracellular domain of the membrane-bound polypeptide comprises a truncated human epidermal growth factor receptor (EGFRt) polypeptide. A truncated EGFRt polypeptide can enable T cell elimination by administering anti-EGFR monoclonal antibody (e.g., cetuximab).

In certain embodiments, the membrane-bound polypeptide comprises a synNotch module. SynNotch modules are disclosed in U.S. Pat. No. 9,670,281 and Morsut et al, Cell, 164, 780-791, 2016, each of which is incorporated by reference in its entirety.

### 2.2. Soluble Polypeptide

In certain embodiments, the soluble polypeptide comprises a dimerization domain that is capable of dimerizing with a dimerization domain comprised in a membrane-bound polypeptide disclosed herein. In certain embodiments, the membrane-bound polypeptide is a membrane-bound polypeptide disclosed herein, e.g., in Section 2.1. In certain embodiments, the dimerization domain comprises a leucin zipper domain. The dimerization domain can be any of the dimerization domains disclosed in Section 2.1.1.

In certain embodiments, the soluble polypeptide comprises a dimerization domain and an antigen-binding domain that is capable of binding to an antigen.

In certain embodiments, the soluble polypeptide comprises a dimerization domain and a cytokine or a chemokine. In certain embodiments, the soluble polypeptide further comprises a tag.

In certain embodiments, the leucine zipper domain of the membrane-bound polypeptide and the leucine zipper domain of the soluble polypeptide are a pair of orthogonal zippers, i.e., they are the specific partners for each other to form heterodimers.

In certain embodiments, the soluble polypeptide further comprises a tag. In certain embodiments, the tag comprises an epitope tag, which comprises an epitope recognized by an antibody. In certain embodiments, the epitope tag is selected from the group consisting of a Myc-tag, a HA-tag, a Flag-tag, a V5-tag, a T7 tag, and combinations thereof. In certain embodiments, the tag comprises an affinity tag that binds to a substrate. In certain embodiments, the affinity tag is selected from the group consisting of a His-tag, a Strep-tag, an E-tag, a streptavidin binding protein tag (SBP-tag), and combinations thereof.

Furthermore, the soluble polypeptide can further comprise a mimotope recognized by a second antibody. Binding of the second antibody to the mimotope can mediates depletion of a cell comprising the membrane-bound polypeptide. In certain embodiments, the mimotope is a CD20 mimotope recognized by an anti-CD20 antibody. In certain embodiments, the anti-CD20 antibody is Rituximab. In certain embodiments, the CD20 mimotope is a circular CD20 mimotope.

In certain embodiments, the CD20 mimotope comprises or has the amino acid sequence set forth in SEQ ID NO: 80.

### 3. Systems Comprising Membrane-Bound Leucine Zipper Polypeptides, Soluble Leucine Zipper Polypeptides and a Plurality of CARs, Safety Switches, Switch Receptors, and/or Cytokines

3.1 Systems Comprising CARs, Safety Switches, Switch Receptors, and/or Cytokines

The presently disclosed subject matter is directed, in certain embodiments, to leucine zipper-based sorting systems comprising a plurality of nucleic acid constructs, wherein the plurality comprises: a first nucleic acid construct encoding a membrane bound polypeptide comprising: (i) an extracellular domain comprising a first leucine zipper sequence; (ii) a transmembrane domain; and (iii) an intracellular domain; as well as a second nucleic acid construct encoding a soluble polypeptide comprising a second leucine zipper sequence capable of heterodimerizing with the first leucine zipper sequence and a signal peptide sequence; wherein each of the first and second nucleic acid constructs further encode one or more CAR, safety switch, switch receptor, and/or cytokine. The one or more CAR, safety switch, and/or switch receptor encoded by the constructs of the present disclosure can themselves comprise a variety of domains, including, but not limited to extracellular domains, transmembrane domains, intracellular domains, and antigen binding domains.

### 3.1.1 CAR, Safety Switch, and Switch Receptor Extracellular, Transmembrane, and Intracellular Domains

In certain embodiments, the one or more CAR, safety switch, and/or switch receptor comprises an extracellular domain. In certain non-limiting embodiments, the extracellular domain comprises an extracellular domain of an immune checkpoint molecule. Non-limiting examples of immune checkpoint molecule include PD-1, Fas, CD200R1, TIGIT, ICOS, CTLA-4, BTLA, TIM-3, LAG-3, leukocyte associated immunoglobulin like receptor (LAIR1), herpesvirus entry mediator (HVEM), 2B4 (CD244), CD160, Galectin9, VISTA, PSGL-1, fragments thereof or combinations thereof.

In certain embodiments, the immune checkpoint molecule is a dominant negative molecule, e.g., a dominant negative receptor (DNR) or variant thereof. In certain embodiments, the dominant negative molecule is selected from the group consisting of inhibitors of immune checkpoint molecules, tumor necrosis factor receptor superfamily (TNFRSF) members, and TGFβ receptors. Non-limiting examples of DNRs include PD-1-DNR, Fas-DNR, CD200R1-DNR, TIGIT-DNR, ICOS-DNR, CTLA-4-DNR, BTLA-DNR, TIM-3-DNR, LAG-3-DNR, LAIR1-DNR, HVEM-DNR, CD244-DNR, CD160-DNR, VISTA-DNR, PSGL-1-DNR, variants thereof, or combinations thereof. Details of dominant negative (DN) forms of inhibitors of an immune checkpoint molecule are disclosed in WO2017/040945 and WO2017/100428, the contents of each of which are incorporated by reference herein in their entireties. In certain embodiments, the extracellular domain further comprises a dominant negative form of an immune checkpoint inhibitor disclosed in WO2017/040945. In certain embodiments, the extracellular domain further comprises a dominant negative form of an immune checkpoint inhibitor disclosed in WO2017/100428.

In some embodiments, the immune checkpoint molecule is PD-1 having an extracellular domain set forth in SEQ ID NO: 52. In some embodiments, the immune checkpoint molecule is CD200R1 having an extracellular domain set forth in SEQ ID NO: 54. In some embodiments, the immune checkpoint molecule is Fas having an extracellular domain set forth in SEQ ID NO: 55.

In certain embodiments, the dominant negative molecule is a PD-1 dominant negative (i.e., PD-1 DNR) molecule. In certain embodiments, the PD-1 DN comprises (a) at least a fragment of an extracellular domain of PD-1 comprising a ligand binding region, and (b) a transmembrane domain.

In certain embodiments, the PD-1 DN is a mouse PD-1 DN. In certain embodiments, the PD-1 DN comprises or has the amino acid sequence set forth in SEQ ID NO: 52.

In certain embodiments, the dominant negative molecule is a CD200R1 dominant negative receptor (i.e., CD200R1-DNR) molecule. In certain embodiments, the CD200R1-DNR comprises (a) at least a fragment of an extracellular domain of CD200R1 comprising a ligand binding region, and (b) a transmembrane domain.

In certain embodiments, the CD200R1-DNR is a mouse CD200R1-DNR. In certain embodiments, the CD200R1-DNR comprises or has the amino acid sequence set forth in SEQ ID NO: 54.

In certain embodiments, the dominant negative molecule is a Fas dominant negative (i.e., Fas-DNR) molecule. In certain embodiments, the Fas-DNR comprises (a) at least a fragment of an extracellular domain of Fas comprising a ligand binding region, and (b) a transmembrane domain.

In certain embodiments, the Fas-DNR is a mouse Fas-DNR. In certain embodiments, the Fas-DNR comprises or has the amino acid sequence set forth in SEQ ID NO: 55.

In certain non-limiting embodiments, the extracellular domain comprises a sequence of at least one co-stimulatory ligand or a fragment thereof. Non-limiting examples of co-stimulatory ligands include tumor necrosis factor (TNF) family members, immunoglobulin (Ig) superfamily members, and a combination thereof. In certain embodiments, the co-stimulatory ligand is selected from the group consisting of tumor necrosis factor (TNF) family members. In certain embodiments, the TNF family member is selected from the group consisting of OX40, CD40, CD30, 4-1BB, CD27, RANK, GITR, LTBR, HVEM, BAFF-R, TACI, BCMA, TROY, CD70, GITRL, CD40L, and CD30L, fragments thereof, or combinations thereof.

In certain embodiments, the intracellular domain comprises a member of the immunoglobulin superfamily. In a non-limiting embodiment, the intracellular domain comprises an intracellular domain of CD28.

In certain embodiments, the extracellular domain further comprises a tag. In certain embodiments, the tag comprises an epitope tag recognized by a first antibody. Non-limiting examples of epitope tags include Myc-tag, a HA-tag, a Flag-tag, a V5-tag, and a T7-tag.

In certain embodiments, the tag comprises an affinity tag that binds to a substrate. Non-limiting examples of affinity tags include a His-tag, a Strep-tag, an E-tag, and a streptavidin binding protein tag (SBP-tag).

Furthermore, the extracellular domain can further comprise a mimotope recognized by a second antibody. Binding of the second antibody to the mimotope can mediates depletion of a cell comprising the membrane-bound polypeptide. In certain embodiments, the mimotope is a CD20 mimotope recognized by an anti-CD20 antibody. In certain embodiments, the anti-CD20 antibody is Rituximab. In certain embodiments, the CD20 mimotope is a circular CD20 mimotope. In certain embodiments, the CD20 mimotope comprises or has the amino acid sequence set forth in SEQ ID NO: 80.

In certain embodiments, the one or more CAR, safety switch, and/or switch receptor comprises a transmembrane domain. In certain embodiments, the transmembrane domain can comprise a CD8 polypeptide (e.g., the transmembrane domain of CD8 or a fragment thereof), a CD28 polypeptide (e.g., the transmembrane domain of CD28 or a fragment thereof), a CD3ζ polypeptide (e.g., the transmembrane domain of CD3ζ or a fragment thereof), a CD4 polypeptide (e.g., the transmembrane domain of CD4 or a fragment thereof), a 4-1BB polypeptide (e.g., the transmembrane domain of 4-1BB or a fragment thereof), an OX40 polypeptide (e.g., the transmembrane domain of OX40 or a fragment thereof), an ICOS polypeptide (e.g., the transmembrane domain of ICOS or a fragment thereof), a CD2 polypeptide (e.g., the transmembrane domain of CD2 or a fragment thereof), a synthetic peptide (not based on a protein associated with the immune response), or a combination thereof.

In certain embodiments, the transmembrane domain comprises a CD8 polypeptide (e.g., the transmembrane domain of CD8 or a fragment thereof). In certain embodiments, the CD8 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001139345.1 (SEQ ID NO: 90) or a fragment thereof, and/or may optionally comprise up to one or up to two or up to three conservative amino acid substitutions. In certain embodiments, the CD8 polypeptide comprises or has an amino acid sequence that is a consecutive fragment of SEQ ID NO: 90, which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 150 to 200, 183 to 203, or 200 to 235 of SEQ ID NO: 90. In certain embodiments, the transmembrane domain of the membrane-bound polypeptide comprises a CD8 polypeptide comprising or having an amino acid sequence of amino acids 183 to 203 of SEQ ID NO: 90.

In certain embodiments, the CD8 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: AAA92533.1 (SEQ ID NO: 91) or a fragment thereof, and/or may optionally comprise up to one or up to two or up to three conservative amino acid substitutions. In certain embodiments, the CD8 polypeptide comprises or has an amino acid sequence that is a consecutive fragment of SEQ ID NO: 91 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, or 200 to 247 of SEQ ID NO: 91.

In certain embodiments, the CD8 polypeptide comprises or has the amino acid sequence set forth in SEQ ID NO: 58.

In certain embodiments, the CD8 polypeptide comprises or has the amino acid sequence set forth in SEQ ID NO: 59.

In certain embodiments, the transmembrane domain comprises a CD28 polypeptide (e.g., the transmembrane domain of CD28 or a fragment thereof). The CD28 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P10747 or NP_006130 (SEQ ID No: 92), or a fragment thereof, and/or may optionally comprise up to one or up to two or up to three conservative amino acid substitutions. In certain embodiments, the CD28 polypeptide comprises or has an amino acid sequence that is a consecutive fragment of SEQ ID NO: 92 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, 153 to 179, or 200 to 220 of SEQ ID NO: 92. In certain embodiments, the transmembrane domain of a presently disclosed membrane-bound polypeptide comprises a CD28 polypeptide comprising or having an amino acid sequence of amino acids 153 to 179 of SEQ ID NO: 92.

In certain embodiments, the transmembrane domain comprises a CD28 polypeptide comprising or having the amino acid sequence set forth in SEQ ID NO: 56.

In certain embodiments, the transmembrane domain comprises a CD28 polypeptide comprising or having the amino acid sequence set forth in SEQ ID NO: 57.

In certain embodiments, the transmembrane domain comprises a CD4 polypeptide (e.g., the transmembrane domain of CD4 or a fragment thereof). The CD4 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_038516.1 (SEQ ID NO: 93) or a fragment thereof, and/or may optionally comprise up to one or up to two or up to three conservative amino acid substitutions. In certain embodiments, the CD4 polypeptide comprises or has an amino acid sequence that is a consecutive fragment of SEQ ID NO: 93 which is at least 20, or at least 30, or at least 40, or at least 50, and up to 457 amino acids in length. Alternatively or additionally, in non-limiting various embodiments, the CD4 polypeptide comprises or has an amino acid sequence of amino acids 1 to 457, 1 to 50, 50 to 100, 100 to 150, 150 to 200, 200 to 250, 250 to 300, 300 to 350, 350 to 400, 395 to 417, or 400 to 457 of SEQ ID NO: 93. In certain embodiments, the transmembrane domain comprises a CD4 polypeptide comprising or having amino acids 395 to 417 of SEQ ID NO: 93. In certain embodiments, the transmembrane domain comprises a CD4 polypeptide (e.g., the transmembrane domain of CD4 or a fragment thereof). The CD4 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_000607.1 (SEQ ID No: 94) or a fragment thereof, and/or may optionally comprise up to one or up to two or up to three conservative amino acid substitutions. In certain embodiments, the CD4 polypeptide comprises or has an amino acid sequence that is a consecutive fragment of SEQ ID NO: 94 which is at least 20, or at least 30, or at least 40, or at least 50, and up to 458 amino acids in length. Alternatively or additionally, in non-limiting various embodiments, the CD4 polypeptide comprises or has an amino acid sequence of amino acids 1 to 457, 1 to 50, 50 to 100, 100 to 150, 150 to 200, 200 to 250, 250 to 300, 300 to 350, 350 to 400, 397 to 418, or 400 to 457 of SEQ ID NO: 94. In certain embodiments, the transmembrane domain comprises a CD4 polypeptide comprising or having amino acids 397 to 418 of SEQ ID NO: 94

In certain embodiments, the one or more CAR, safety switch, and/or switch receptor comprises an intracellular domain. In certain non-limiting embodiments, the intracellular domain provides an activation signal to a cell (e.g., a cell of the lymphoid lineage, e.g., a T cell). In certain embodiments, the intracellular domain comprises an immune activating molecule. In certain embodiments, the immune activating molecule is a CD3ζ polypeptide.

In certain non-limiting embodiments, the intracellular domain comprises a CD3ζ polypeptide or a fragment thereof. CD3ζ can activate or stimulate a cell. CD3ζ comprises 3 ITAMs and transmits an activation signal to the cell (e.g., a cell of the lymphoid lineage, e.g., a T cell) after antigen is bound. The intracellular signaling domain of the CD3ζ-chain is the primary transmitter of signals from endogenous TCRs. In certain embodiments, the CD3ζ polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_932170 (SEQ ID No: 95), or a fragment thereof, and/or may optionally comprise up to one or up to two or up to three conservative amino acid substitutions. In certain non-limiting embodiments, the CD3ζ polypeptide comprises or has an amino acid sequence that is a consecutive fragment of SEQ ID NO: 95, which is at least 20, or at least 30, or at least 40, or at least 50, and up to 164 amino acids in length. Alternatively or additionally, in non-limiting various embodiments, the CD3ζ polypeptide comprises or has an amino acid sequence of amino acids 1 to 164, 1 to 50, 50 to 100, 52 to 164, 100 to 950, or 950 to 164 of SEQ ID NO: 95. In certain embodiments, the CD3ζ polypeptide comprises or has an amino acid sequence of amino acids 52 to 164 of SEQ ID NO: 95.

In certain embodiments, the CD3ζ polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001106864.2 (SEQ ID No: 96) or a fragment thereof, and/or may optionally comprise up to one or up to two or up to three conservative amino acid substitutions. In certain non-limiting embodiments, the CD3ζ polypeptide comprises or has an amino acid sequence that is a consecutive fragment of SEQ ID NO: 96, which is at least about 20, or at least about 30, or at least about 40, or at least about 50, or at least about 90, or at least about 100, and up to 188 amino acids in length. Alternatively or additionally, in non-limiting various embodiments, the CD3ζ polypeptide comprises or has an amino acid sequence of amino acids 1 to 164, 1 to 50, 50 to 100, 100 to 150, or 150 to 188 of SEQ ID NO: 96.

In certain embodiments, the CD3ζ polypeptide comprises or has an amino acid sequence set forth in SEQ ID NO: 66.

In certain embodiments, the intracellular domain comprises a murine CD3ζ polypeptide.

In certain embodiments, the intracellular domain comprises a human CD3ζ polypeptide.

In certain non-limiting embodiments, the intracellular domain provides an activation signal and a stimulation signal to a cell. In certain embodiments, the domain comprises at least one costimulatory molecule or a fragment thereof.

In certain embodiments, the at least one co-stimulatory signaling region comprises a CD28 polypeptide (e.g., the intracellular domain of CD28 or a fragment thereof), a 4-1BB polypeptide (e.g., the intracellular domain of 4-1BB or a fragment thereof), an OX40 polypeptide (e.g., the intracellular domain of OX40 or a fragment thereof), an ICOS polypeptide (e.g., the intracellular domain of ICOS or a fragment thereof), a DAP-10 polypeptide (e.g., the intracellular domain of DAP-10 or a fragment thereof), or a combination thereof. In certain embodiments, the at least one co-stimulatory signaling region comprises a CD28 polypeptide. In certain embodiments, the at least one co-stimulatory signaling region comprises an intracellular domain of CD28 or a fragment thereof.

In certain embodiments, the costimulatory molecule is a CD28 polypeptide (e.g., the intracellular domain of CD28 or a fragment thereof). The CD28 polypeptide can comprise or have an amino acid sequence that is at least about 80%, at least about 85%, at least about 90%, at least about 95%, at least about 96%, at least about 97%, at least about 98%, at least about 99% or at least 100% homologous or identical to the sequence having a NCBI Reference No: P10747 or NP_006130 (SEQ ID NO: 14) or a fragment thereof, and/or may optionally comprise up to one or up to two or up to three conservative amino acid substitutions. In certain embodiments, the CD28 polypeptide comprises or has an amino acid sequence that is a consecutive fragment of SEQ ID NO: 92 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 150 to 200, or 200 to 220 of SEQ ID NO: 92. In certain embodiments, the CD28 polypeptide comprises or has an amino acid sequence of amino acids 181 to 220 of SEQ ID NO: 92.

In certain embodiments, the CD28 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_031668.3 (SEQ ID NO: 16) or a fragment thereof, and/or may optionally comprise up to one or up to two or up to three conservative amino acid substitutions. In certain embodiments, the CD28 polypeptide comprises or has an amino acid sequence that is a consecutive fragment of SEQ ID NO: 97 which is at least about 20, or at least about 30, or at least about 40, or at least about 50, and up to 218 amino acids in length. Alternatively or additionally, in non-limiting various embodiments, the CD28 polypeptide comprises or has an amino acid sequence of amino acids 1 to 218, 1 to 50, 50 to 100, 100 to 150, 150 to 200, or 200 to 218 of SEQ ID NO: 97. In certain embodiments, the CD28 polypeptide comprises or has an amino acid sequence of amino acids 178 to 218 of SEQ ID NO: 97.

In certain embodiments, the costimulatory molecule is a mouse CD28 polypeptide. In certain embodiments, the costimulatory molecule is a human CD28 polypeptide.

In certain embodiments, the intracellular domain comprises two costimulatory molecules, e.g., CD28 and 4-1BB or CD28 and OX40.

In certain embodiments, the at least one co-stimulatory signaling region comprises a 4-1BB polypeptide. In certain embodiments, the at least one co-stimulatory signaling region comprises an intracellular domain of 4-1BB or a fragment thereof.

In certain embodiments, the costimulatory molecule is a 4-1BB polypeptide (e.g., the intracellular domain of 4-1BB or a fragment thereof). 4-1BB can act as a tumor necrosis factor (TNF) ligand and have stimulatory activity. The 4-1BB polypeptide can comprise or have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P41273 or NP_001552 (SEQ ID NO: 98) or a fragment thereof, and/or may optionally comprise up to one or up to two or up to three conservative amino acid substitutions.

In accordance with the presently disclosed subject matter, a “4-1BB nucleic acid molecule” refers to a polynucleotide encoding a 4-1BB polypeptide.

In certain embodiments, the intracellular domain comprises a member of the TNFRSF.

In certain embodiments, the intracellular domain comprises an intracellular domain of one or more co-stimulatory signaling domains. Non-limiting examples of co-stimulatory signaling domains include intracellular domain of OX40, CD40, RANK, GITR, LTBR, HVEM, BAFF-R, TACI, BCMA, TROY, CD30, 4-1BB, CD27, RANK, GITR, LTBR, HVEM, BAFF-R, TACI, BCMA, TROY, fragments thereof, or combinations thereof.

In certain embodiments, the intracellular domain comprises a member of the immunoglobulin superfamily. In a non-limiting embodiment, the intracellular domain comprises an intracellular domain of CD28.

In certain embodiments, the system encodes a switch receptor wherein the extracellular domain comprises an inhibitor molecule (e.g., immune checkpoint molecule) or fragment thereof that is attached via a transmembrane domain to an intracellular stimulatory molecule, whereby binding of an inhibitory ligand to the inhibitory molecule on the cell surface switches the signaling into a stimulatory signal inside the cell. Non-limiting examples of such switch receptors include PD-1-OX40, Fas-4-1BB (FasBB), CD200R1-CD27, TIGIT-4-1BB (TIGITBB), ICOS-CD27, variants thereof, or combinations thereof.

In certain embodiments, the costimulatory molecule is an OX40 polypeptide (e.g., the intracellular domain of OX40 or a fragment thereof). The OX40 polypeptide can comprise or have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P43489 or NP_003318 (SEQ ID NO: 99) or a fragment thereof, and/or may optionally comprise up to one or up to two or up to three conservative amino acid substitutions.

In accordance with the presently disclosed subject matter, an “OX40 nucleic acid molecule” refers to a polynucleotide encoding an OX40 polypeptide.

In certain embodiments, the at least one co-stimulatory signaling region comprises an ICOS polypeptide. In certain embodiments, the at least one co-stimulatory signaling region comprises an intracellular domain of ICOS or a fragment thereof.

In certain embodiments, the costimulatory molecule is an ICOS polypeptide (e.g., the intracellular domain of ICOS or a fragment thereof). The ICOS polypeptide can comprise or have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_036224 (SEQ ID NO: 100) or a fragment thereof, and/or may optionally comprise up to one or up to two or up to three conservative amino acid substitutions.

In accordance with the presently disclosed subject matter, an “ICOS nucleic acid molecule” refers to a polynucleotide encoding an ICOS polypeptide.

In certain embodiments, the at least one co-stimulatory signaling region comprises two costimulatory molecules or fragments thereof. In certain embodiments, the at least one co-stimulatory signaling region comprises a CD28 polypeptide (e.g., an intracellular domain of CD28 or a fragment thereof) and a 4-1BB polypeptide (e.g., an intracellular domain of 4-1BB or a fragment thereof).

In certain non-limiting embodiments, the intracellular domain alone does not provide an activation signal to a cell. In certain embodiments, the intracellular domain does not comprise a costimulatory molecule. In certain embodiments, the intracellular domain does not comprise a CD3zeta polypeptide.

In certain embodiments, the intracellular domain comprises a CD3 domain, a costimulatory domain, a suicide gene product, survival gene product, fragments thereof, or combinations thereof.

In certain embodiments, the intracellular domain comprises a suicide gene. Suitable suicide genes include, but are not limited to, Herpes simplex virus thymidine kinase (hsv-tk), and inducible Caspase 9 Suicide gene (iCaspase9 or iCasp9).

In certain embodiments, the iCasp9 comprises a sequence as set forth in SEQ ID NO: 88

In certain embodiments, the intracellular domain comprises a survival gene. In certain embodiments, the survival gene product is a caspase resistant Bcl-2.

In certain embodiments, the caspase resistant Bcl-2 comprises a sequence as set forth in SEQ ID NO: 89

In certain embodiments, the intracellular domain comprises a truncated human epidermal growth factor receptor (EGFRt) polypeptide. A truncated EGFRt polypeptide can enable T cell elimination by administering anti-EGFR monoclonal antibody (e.g., cetuximab).

In certain embodiments, the one or more CAR, safety switch, and/or switch receptor comprises a synNotch module. SynNotch modules are disclosed in U.S. Pat. No. 9,670,281 and Morsut et al, Cell, 164, 780-791, 2016, each of which is incorporated by reference in its entirety

### 3.1.2 Antigen Binding Domain

In certain embodiments, the one or more CAR, safety switch, and/or switch receptor comprise an antigen binding domain. In certain embodiments, the antigen binding domain comprises a single-chain variable fragment (scFv), a soluble ligand, a cytokine, or a non-scFv-based antigen recognition motif, or a combination thereof.

In certain non-limiting embodiments, the antigen binding domain (embodied, for example, an scFv or an analog thereof) binds to an antigen with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹ M or less. In certain non-limiting embodiments, the K_{d} is about 3×10⁻⁹ M or less. In certain non-limiting embodiments, the K_{d} is from about 1×10⁻⁹ M to about 3×10⁻⁷ M. In certain non-limiting embodiments, the K_{d} is from about 1.5×10⁻⁹ M to about 3×10⁻⁷ M.

Binding of the antigen binding domain (for example, in an scFv or an analog thereof) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, the antigen binding domain is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and Ypet).

In certain embodiments, the antigen binding domain specifically binds to an antigen. In certain embodiments, the antigen binding domain is an scFv. In certain embodiments, the scFv is a human scFv. In certain embodiments, the scFv is a humanized scFv. In certain embodiments, the scFv is a murine scFv. In certain embodiments, the antigen-binding domain is a Fab, which is optionally crosslinked. In certain embodiments, the antigen-binding domain is a F(ab)₂. In certain embodiments, any of the foregoing molecules may be comprised in a fusion protein with a heterologous sequence to form the antigen-binding domain. In certain embodiments, the scFv is identified by screening scFv phage library with an antigen-Fc fusion protein. In certain embodiments, the antigen is a tumor antigen. In certain embodiments, the antigen is a pathogen antigen.

### 3.1.3 Antigens

In certain embodiments, the antigen binding domain of the one or more CAR, safety switch, and/or switch receptor binds to a tumor antigen. Any tumor antigen can be used in the tumor-related embodiments described herein. The antigen can be expressed as a peptide or as an intact protein or fragment thereof. The intact protein or a fragment thereof can be native or mutagenized. Non-limiting examples of tumor antigens include CD19, CD70, IL1RAP, ABCG2, AchR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, ZDHHC11, CD2, CD3, CD4, CD5, CD19, VpreB, CD40, CD79a, CD70b, CLL-1/CLEC12A, IL-3R complex, TIM-3, TACI, SLAMF7, CD244, E-cadherin, B7-H4, carbonic anhydrase IX (CalX), carcinoembryonic antigen (CEA), an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AchR), Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), k-light chain, kinase insert domain receptor (KDR), L1 cell adhesion molecule (LiCAM), 1 (MAGE-A1), Proteinase3 (PR1), cancer-testis antigen NY-ESO-1, tumor-associated glycoprotein 72 (TAG-72), and Wilms tumor protein (WT-1), PRAME and ERBB variants thereof, or combinations thereof.

In certain embodiments, the antigen binding domain of the one or more CAR, safety switch, and/or switch receptor binds to a human CD19 polypeptide. In certain embodiments, the antigen binding domain of the one or more CAR, safety switch, and/or switch receptor binds to the extracellular domain of a human CD19 protein.

In certain embodiments, the antigen binding domain of the one or more CAR, safety switch, and/or switch receptor binds to an immune checkpoint molecule. Non-limiting example of immune checkpoint molecules include CD200R1, HVEM, Galectin9, VISTA, PSGL-1, PD-1, CTLA-4, CD200R1, TIM-3, Lag-3 and TIGIT.

In certain embodiments, the antigen binding domain of the one or more CAR, safety switch, and/or switch receptor binds to an activating receptor, wherein the binding of the antigen binding domain to the activating receptor is capable of activating an antigen presenting cell (APC). Non-limiting example of immune checkpoint molecules include CD40, Toll Like Receptors (TLRs), FLT3, RANK, and GM-CSF receptor.

In certain embodiments, the antigen binding domain of the one or more CAR, safety switch, and/or switch receptor binds to a biomarker of a hematopoietic lineage cell. Non-limiting example of immune checkpoint molecules include CD3, CD16, CD33, c-Kit, CD161, CD19, CD20, VpPreB, luteinizing hormone receptor (LHCGR), CD123, IL-3R complex, CLEC12A/CLL-1.

In certain embodiments, the antigen binding domain of the one or more CAR, safety switch, and/or switch receptor binds to a pathogen antigen, e.g., for use in treating and/or preventing a pathogen infection or other infectious disease, for example, in an immunocompromised subject. Non-limiting examples of pathogens include a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria and/or fungi include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Aspergillus species and Actinomyces israelli.

### 3.2. Functional Sorting Zip Construct Comprising a Blocking Spacer

The presently disclosed subject matter provides a system, comprising: a) a membrane-bound polypeptide disclosed herein (e.g., a membrane-bound polypeptide comprising: i) a transmembrane domain, ii) an intracellular domain, and iii) an extracellular domain that comprises a first dimerization domain and a blocking spacer), and b) a soluble polypeptide disclosed herein (e.g., a soluble polypeptide comprising i) a second dimerization domain that is capable of dimerizing with the first dimerization domain, and ii) a tag). In certain embodiments, each of the first dimerization domain and the second dimerization domain comprises a leucine zipper domain, and the blocking spacer prevents dimerization of the membrane-bound polypeptide with the soluble polypeptide when the membrane-bound polypeptide and the soluble polypeptide are not expressed from the same cell.

In certain embodiments, the blocking spacer has a length of no more than about 25 amino acids. In certain embodiments, the blocking spacer has a length of between about 5 amino acids and about 25 amino acids. In certain embodiments, the blocking spacer is a truncated CD28 spacer or an IgG1 hinge.

### 3.3. Functional Sorting Zip Construct Comprising a Self-Blocking Feature

The presently disclosed subject matter provides a system, comprising: a) a membrane-bound polypeptide disclosed herein (e.g., a membrane-bound polypeptide comprising: i) a transmembrane domain, ii) an intracellular domain, and iii) an extracellular domain that comprises a first dimerization domain and a second dimerization domain that is capable of dimerizing with the first dimerization domain), and b) a soluble polypeptide disclosed herein (e.g., a soluble polypeptide comprising i) a third dimerization domain that is capable of dimerizing with the first dimerization domain, and ii) a tag). In certain embodiments, each of the first dimerization domain and the second dimerization domain comprises a leucine zipper domain.

In certain embodiments, the antigen is selected from the group consisting of tumor antigens, pathogen antigens, immune checkpoint molecules, activating receptors, and biomarkers of a hematopoietic lineage cell.

### 3.3.1. Functional Sorting Zip Construct Targeting Tumor Antigens

The presently disclosed subject matter provides systems for targeting tumor antigens comprising any membrane-bound polypeptide and/or any soluble polypeptide disclosed herein, or any system disclosed herein. In certain embodiments, the antigen of the antigen binding domain of the soluble polypeptide disclosed herein, optionally comprised in any system disclosed herein, is a tumor antigen. In certain embodiments, the tumor antigen is selected from the group consisting of CD2, CD3, CD4, CD5, CD7, CD8, CD19, CD20, CD22, VpreB, CD79a, CD79b, CD30, CD33, CD38, CD40, CD44v6, CD70, CLL-1/CLEC12A, CD123, IL-3R complex, TIM-3, BCMA, TACI, SLAMF7, CD244, Epcam, E-cadherin, and fragments or combinations thereof.

### 3.3.2. Functional Sorting Zip Construct for Activating APCs

The presently disclosed subject matter provides systems for activating APCs comprising any membrane-bound polypeptide and/or any soluble polypeptide disclosed herein, or any system disclosed herein. In certain embodiments, the antigen of the antigen binding domain of the soluble polypeptide, optionally comprised in any system disclosed herein, is an activating receptor. In certain embodiments, the binding of the antigen binding domain to the activating receptor is capable of activating an antigen presenting cell (APC). In certain embodiments, the APC is a professional APC. In certain embodiments, the professional APC is selected from the group consisting of dendritic cells, macrophages, B cells, and combinations thereof. In certain embodiments, the APC is a non-professional APC. In certain embodiments, the APC is a cell of the myeloid lineage. In certain embodiments, the cell of myeloid lineage is selected from the group consisting of dendritic cells, macrophages, monocytes and combinations thereof. In certain embodiments, the activating receptor is selected from the group consisting of CD40, Toll Like Receptors (TLRs), FLT3, RANK, GM-CSF receptor, and fragments or combinations thereof.

### 3.3.3. Functional Sorting Zip Construct Targeting Immune Checkpoint Blockers

The presently disclosed subject matter provides systems for targeting immune checkpoint blockers comprising any membrane-bound polypeptide and/or any soluble polypeptide disclosed herein, or any system disclosed herein. In certain embodiments, the antigen of the antigen binding domain of the soluble polypeptide disclosed herein, optionally comprised in any system disclosed herein, is an immune checkpoint molecule. In certain embodiments, binding of the antigen binding domain to the immune checkpoint molecule is capable of blocking an immune checkpoint signal in an immunoresponsive cell. In certain embodiments, the immune checkpoint molecule is selected from the group consisting of CD200R1, HVEM, Galectin9, PD-1, CTLA-4, CD200R1, TIM-3, Lag-3, TIGIT, VISTA, PSGL-1, and fragments or combinations thereof.

### 3.3.4. Functional Sorting Zip Construct for Conditioning Regimen for Hematopoietic Stem Cell Transplantation

The presently disclosed subject matter provides systems comprising any membrane-bound polypeptide and/or any soluble polypeptide disclosed herein, or any system disclosed herein. In certain embodiments, the antigen of the antigen binding domain of the soluble polypeptide disclosed herein, optionally comprised in any system disclosed herein, is a biomarker of a hematopoietic lineage cell. In certain embodiments, the biomarker of a hematopoietic lineage cell is selected from the group consisting of CD3, CD16, CD33, c-Kit, CD161, CD19, CD20, vPreB/CD179a (preB cell receptor), luteinizing hormone receptor (LHCGR), CD123, IL-3R complex, CLEC12A/CLL-1, and combinations thereof. In certain embodiments, the system comprises at least four soluble polypeptides, wherein the antigen binding domain of each soluble polypeptide binds to a different biomarker of a hematopoietic lineage cell. In certain embodiments, each of the at least four soluble polypeptides comprises a dimerization domain that comprises a leucine zipper domain. In certain embodiments, the system comprises a first soluble polypeptide that binds to CD3, a second soluble polypeptide that binds to CD19, a third soluble polypeptide that binds to CD161 and a fourth soluble polypeptide that binds to c-Kit.

In certain embodiments, the dimerization domain that interacts with the membrane-bound polypeptide (also referred to as “capture leucine zipper” or “transmembrane capture leucine zipper”) is the appended heterodimerizing leucine zipper of the soluble polypeptide (also referred to as “secreted leucine zippers” or “secreted molecules”), which also comprise an affinity tag. The membrane-bound polypeptide and soluble polypeptides may have the same heterodimerizing leucine zipper domain (e.g., RR12EE345L) if interacting with the same heterodimerizing leucine zipper domain (e.g., EE12RR345L). In certain embodiments, the leucine zipper of the multiple soluble polypeptides (e.g., four soluble polypeptides) and the leucine zipper of the membrane-bound polypeptide are non-orthogonal zippers, i.e., degenerate zippers, which allow all the antigens to be targeted by a membrane-bound polypeptide (a CD3ζ comprising ZipR-CAR), which can result in killing of that component of the hematopoietic system by the effector cells.

### 3.3.5. Functional Sorting Zip Construct Comprising AND-CAR

The presently disclosed subject matter provides systems comprising any membrane-bound polypeptide and/or any soluble polypeptide disclosed herein, or any system disclosed herein. In certain embodiments, the system further comprises c) a chimeric antigen receptor (CAR) comprising a second antigen binding domain (e.g., one that binds to a second antigen), a transmembrane domain, and an intracellular activating domain. The CAR can activate an immunoresponsive cell, e.g., a T cell. In certain embodiments, the system further comprises an inhibitory receptor comprising a leucine zipper domain, wherein the inhibitory receptor binds to a third antigen, e.g., the inhibitory receptor comprising a third antigen binding domain that binds to a third antigen. The inhibitory receptor can be membrane-bound. The inhibitory receptor can be a tyrosine phosphatase-based inhibitory receptor. In certain embodiments, the tyrosine phosphatase is selected from the group consisting of PTPRJ, PTPRC, PTPN22, and PTPN6. In the absence of the third antigen, the inhibitory receptor constitutively inhibits and/or deactivates the CAR, e.g., by dephosphorylation. Binding of the inhibitory receptor to the third antigen prevents the inhibition and/or deactivation of the CAR by the inhibitory receptor, e.g., upon binding of the inhibitory receptor, the inhibitory receptor does not inhibit the CAR. In summary, the inhibitory receptor is constitutively inhibitory, but can be “turned off” in the presence of the third antigen (e.g., the CAR inhibitory capacity of the inhibitory receptor is turned off).

3.4. Zip Construct Comprising Membrane Bound Cytokines and/or Chemokines

The presently disclosed subject matter provides a system comprising: a) a membrane-bound polypeptide disclosed herein (e.g., a membrane-bound polypeptide comprising a transmembrane domain, an intracellular domain and an extracellular domain that comprises a first dimerization domain), and b) a soluble polypeptide (e.g., a soluble polypeptide comprising a second dimerization domain that is capable of dimerizing with the first dimerization domain, and a cytokine or a chemokine). In certain embodiments, each of the first dimerization domain and the second dimerization domain comprises a leucine zipper domain.

### 3.5. Exemplary Systems

In certain embodiments, the membrane-bound polypeptide is RR12EE345L linker EE12RR345L Thy1.1 P2A CD20 CD8H CD8TM CD28z E2A. In certain embodiments, the membrane-bound polypeptide comprises SEQ ID NO: 105.

In certain embodiments, the membrane-bound polypeptide is RR12EE345L linker EE12RR345L Thy1.1 P2A CD20 CD28H CD28TM CD28z E2A. In certain embodiments, the membrane-bound polypeptide comprises SEQ ID NO: 106.

In certain embodiments, the membrane-bound polypeptide is Q2-RR12EE345L P2A iC9 F2A 3×FLAG BCL2 D34A E2A CD19 CD8H CD8TM CD28z. In certain embodiments, the membrane-bound polypeptide comprises SEQ ID NO: 107.

In certain embodiments, the membrane-bound polypeptide is Q2-RR12EE345L P2A iC9 F2A CD19 CD8H CD8TM CD28z. In certain embodiments, the membrane-bound polypeptide comprises SEQ ID NO: 108.

In certain embodiments, the membrane-bound polypeptide is R2 3N PD-1 DNR P2A CD20 CD28H CD28TM CD28z E2A Fas-DNR. In certain embodiments, the membrane-bound polypeptide comprises SEQ ID NO: 109.

In certain embodiments, the membrane-bound polypeptide is 3N Thy1.1 P2A CD20 CD28H CD28TM CD28z E2A. In certain embodiments, the membrane-bound polypeptide comprises SEQ ID NO: 110.

In certain embodiments, the membrane-bound polypeptide is Q2-RR12EE345L P2A iC9 F2A. In certain embodiments, the membrane-bound polypeptide comprises SEQ ID NO: 111.

In certain embodiments, the membrane-bound polypeptide is 3N PD-1 CD28TM CD28z (PD-1 CAR). In certain embodiments, the membrane-bound polypeptide comprises SEQ ID NO: 112.

In certain embodiments, the membrane-bound polypeptide is R2 3N PD-1 DNR P2A CD20 CD28H CD28TM CD28z E2A FasBB. In certain embodiments, the membrane-bound polypeptide comprises SEQ ID NO: 113.

In certain embodiments, the membrane-bound polypeptide is 3N Thy1.1 P2A CD20 CD28H CD28TM CD28z E2A FasBB. In certain embodiments, the membrane-bound polypeptide comprises SEQ ID NO: 114.

In certain embodiments, the membrane-bound polypeptide is Q2-RR12EE345L P2A CD8SP R2 PD-1H delta F2A CD200R1 CD27 E2A CD19 CD8H CD8TM CD28z. In certain embodiments, the membrane-bound polypeptide comprises SEQ ID NO: 115.

In certain embodiments, the membrane-bound polypeptide is R2 3N PD-1 OX40 P2A CD20 CD28H CD28TM CD28z E2A FasBB. In certain embodiments, the membrane-bound polypeptide comprises SEQ ID NO: 116 In certain embodiments, the membrane-bound polypeptide is Q2-RR12EE345L P2A R2 BAFF-R R2 CD28H CD28TM CD28 1XX E2A CD79b FLAG PD-1H CD8TM CD28 1XX. In certain embodiments, the membrane-bound polypeptide comprises SEQ ID NO: 117.

In certain embodiments, the membrane-bound polypeptide is 3N Thy1.1 P2A CD20 CD8H CD8TM CD28 1XX. In certain embodiments, the membrane-bound polypeptide comprises SEQ ID NO: 118.

In certain embodiments, the membrane-bound polypeptide is R2 3N PD-1 OX40 P2A CD20 CD8H CD8TM CD28 1XX E2A FasBB. In certain embodiments, the membrane-bound polypeptide comprises SEQ ID NO: 119.

In certain embodiments, the membrane-bound polypeptide is 334354 hCD19 VHH myc CD8H CS CD8TM CD28 1XX E2A 2MC57 VHH CD20 ST CD8H CD8TM CD28 1XX P2A V5 3N PD-1H delta. In certain embodiments, the membrane-bound polypeptide comprises SEQ ID NO: 120.

In certain embodiments, the membrane-bound polypeptide is 334354 hCD19 VHH myc CD8H CS CD8TM CD28 1XX E2A 2MC57 VHH CD20 ST CD8H CD8TM CD28 1XX P2A R2 3N CD200R1 CD27. In certain embodiments, the membrane-bound polypeptide comprises SEQ ID NO: 121.

In certain embodiments, the membrane-bound polypeptide is IL-2SP PD-1 OX40 P2A FasBB. In certain embodiments, the membrane-bound polypeptide comprises SEQ ID NO: 122.

### 4. Cells

The presently disclosed subject matter provides cells comprising a membrane-bound polypeptide, a soluble polypeptide and/or a system disclosed herein. In certain embodiments, the polypeptides and/or the system are capable of activating or inhibiting an immunoresponsive cell. In certain embodiments, the polypeptides and/or the system are capable of promoting an anti-tumor effect of an immunoresponsive cell. The cells can be transduced with the polypeptides and/or the systems such that the cells co-express the polypeptides and/or the system. In certain embodiments, the cell is an immunoresponsive cell. The cell can be a cell of the lymphoid lineage or a myeloid lineage.

Cells of the lymphoid lineage can produce antibodies, regulate the cellular immune system, detect foreign agents in the blood, and detect cells foreign to the host, and the like. Non-limiting examples of cells of the lymphoid lineage include T cells, B cells, dendric cells, Natural Killer (NK) cells, cells from which lymphoid cells may be differentiated. In certain embodiments, the stem cell is a pluripotent stem cell. In certain embodiments, the pluripotent stem cell is an embryonic stem cell or an induced pluripotent stem cell.

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), Natural killer T cells, Mucosal associated invariant T cells, and T6 T cells. Cytotoxic T cells (CTL or killer T cells) are a subset of T lymphocytes capable of inducing the death of infected somatic or tumor cells. A patient's own T cells may be genetically modified to target specific antigens through the introduction of any polypeptide or system disclosed herein. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a Natural killer cell. Natural killer (NK) cells can be lymphocytes that are part of cell-mediated immunity and act during the innate immune response. NK cells do not require prior activation in order to perform their cytotoxic effect on target cells.

In certain embodiments, the cells are human lymphocytes. In certain embodiments, the human lymphocytes comprise without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and 3 heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The cells (e.g., T cells) can be autologous, non-autologous (e.g., allogeneic), or derived in vitro from engineered progenitor or stem cells.

In certain embodiments, the cells of are cells of the myeloid lineage. In certain embodiments, the cells of the myeloid lineage comprise, without limitation, monocytes, macrophages, basophils, neutrophils, eosinophils, mast cell, erythrocyte, and thrombocytes.

The presently disclosed cells are capable of modulating the tumor microenvironment. Tumors have a microenvironment that suppresses the host immune response using any of a series of mechanisms by malignant cells to protect themselves from immune surveillance, recognition and elimination. Immune suppressive factors include but are not limited to infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred modified T cells (e.g., CAR T cells) upon encounter with targeted tumor cells.

In certain embodiments, the presently disclosed cells have increased cell persistence. In certain embodiments, the presently disclosed cells have decreased apoptosis and/or anergy.

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. Monoclonal antibodies (mAbs) are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In certain embodiments, at least about 80%, usually at least 70% of the total hematopoietic cells are removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be distinguished from dead cells, by employing dyes associated with dead cells such as propidium iodide (PI). In certain embodiments, the cells are collected in a medium comprising 2% fetal calf serum (FCS) or 0.2% bovine serum albumin (BSA) or any other suitable, e.g., sterile, isotonic medium.

The presently disclosed subject matter encompasses, in certain embodiments, engineered immune cells comprising a system as disclosed herein. In certain embodiments, the engineered immune cell is a T cell.

### 5. Nucleic Acid Compositions and Vectors

Genetic modification of a cell (e.g., a T cell) can be accomplished by transducing a substantially homogeneous cell composition with a recombinant DNA construct. In certain embodiments, a retroviral vector is employed for the introduction of the DNA construct into the cell. For example, a polynucleotide encoding any polypeptide or system disclosed herein can be cloned into a retroviral vector and expression can be driven from its endogenous promoter, from the retroviral long terminal repeat, or from a promoter specific for a target cell type of interest. In certain embodiments, the retroviral vector is a gammaretroviral vector. In certain embodiments, the retroviral vector is a lentiviral vector. Non-viral vectors may be used as well.

For initial genetic modification of a cell to include a polypeptide and/or a system disclosed herein, a retroviral vector is generally employed for transduction, however any other suitable viral vector or non-viral delivery system can be used. The polypeptides and/or the system can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins are functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits a high efficiency of infection, stable integration into the host cell genome, and durable expression of the recombinant gene product(s) (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALENs nucleases, CRISPR). Transient expression may be obtained by RNA electroporation. In certain embodiments, recombinant receptors can be introduced by a transposon-based vector. In certain embodiments, the transposon-based vector comprises a transposon (a.k.a. a transposable element). In certain embodiments, the transposon can be recognized by a transposase. In certain embodiments, the transposase is a Sleeping Beauty transposase.

The resulting cells can be grown under conditions similar to those for unmodified cells, whereby the modified cells can be expanded and used for a variety of purposes.

### 6. Polypeptides and Analogs

The presently disclosed subject matter provides methods for improving the activity of an amino acid sequence or nucleic acid sequence by introducing an alteration in the sequence. Such alterations may include certain mutations, deletions, insertions, or post-translational modifications. The presently disclosed subject matter further comprises, in certain embodiments, analogs of any naturally occurring polypeptide disclosed herein (including, but not limited to, CD8, CD28, CD80, 4-1BBL, and CD3z). Analogs can differ from a naturally occurring polypeptide disclosed herein by amino acid sequence differences, by post-translational modifications, or by both. Analogs can exhibit at least about 80%, at least about 85%, at least about 90%, at least about 95%, at least about 96%, at least about 97%, at least about 98%, at least about 99% or at least about 100% homologous to all or part of a naturally occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs can have a chemical structure designed to mimic the functional activity of a protein/peptide disclosed herein. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in a cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 7. Administration

Compositions comprising the presently disclosed cells can be provided systemically or directly to a subject for inducing and/or enhancing an immune response to an antigen and/or treating and/or preventing a neoplasia, pathogen infection, or infectious disease. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells, NK cells, or CTL cells in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus). At least about 1×10⁵ cells are administered, eventually reaching about 1×10¹⁰ or more. The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed cells and a pharmaceutically acceptable carrier. Administration can be autologous or heterologous. For example, cells can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 8. Formulations

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that achieves the selected viscosity. The choice of suitable carriers and other additives depend on the exact route of administration and the nature of the particular dosage form, e.g., liquid dosage form (e.g., whether the composition is to be formulated into a solution, a suspension, gel, or another liquid form, such as a time release form or liquid-filled form).

The quantity of cells to be administered varies for the subject being treated. In a one embodiment, between about 10⁴ and about 10¹⁰, between about 10⁵ and about 10⁹, or between about 10⁶ and about 10⁸ of the presently disclosed cells are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein and, the time for sequential administrations can be ascertained without undue experimentation.

### 9. Methods of Treatment

The presently disclosed subject matter provides methods for treating a disease comprising providing to a subject in need thereof a population of modified cells comprising the system disclosed herein; or a cell modified according to the method disclosed herein; or an enriched population of cells according to the method disclosed herein.

In certain embodiments, the method further comprises culturing the T cells with a small molecular weight inhibitor before administering to the subject. In certain embodiments, the small molecular weight inhibitor is a Src kinase inhibitor. In certain embodiments, the Src inhibitor is Dasatinib.

In certain embodiments, the subject is a human subject.

In certain embodiments, the disease is a cancer, an autoimmune disease, an inflammatory disease, or a graft versus-host disease.

In certain embodiments, the disease is a cancer, an autoimmune disease, an inflammatory disease, or a graft versus-host disease. Non-limiting examples of cancer include cancer is leukemia, lymphoma, myeloma, ovarian cancer, breast cancer, bladder cancer, brain cancer, colon cancer, intestinal cancer, liver cancer, lung cancer, pancreatic cancer, prostate cancer, testicular cancer, anal cancer, skin cancer, stomach cancer, glioblastoma, throat cancer, melanoma, neuroblastoma, adenocarcinoma, glioma, or soft tissue sarcoma.

In certain embodiments, the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia.

In certain embodiments, the lymphoma is Hodgkin's lymphoma or non-Hodgkin's lymphoma.

In certain embodiments, the non-Hodgkin's lymphoma is B-cell non-Hodgkin's lymphoma or T-cell non-Hodgkin's lymphoma.

In certain embodiments, the cancer comprises cells expressing BAFF-R, CD79, CD70, CD19, CD20, PD-1, Tim-3, variants thereof or combinations thereof.

In certain embodiments, the cancer comprises cells expressing at least one antigen selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, and ZDHHC11.

The method comprises administering to a subject an effective amount of the cells disclosed herein or a pharmaceutical composition comprising such cells. The presently disclosed cells and compositions comprising thereof can be used for treating and/or preventing neoplasia in a subject. The presently disclosed cells and compositions comprising thereof can be used for prolonging the survival of a subject suffering from a neoplasm. The presently disclosed cells and compositions comprising thereof can also be used for treating and/or preventing a pathogen infection or other infectious disease in a subject, such as an immunocompromised human subject. Such methods comprise administering an amount effective the presently disclosed cells or a composition (e.g., a pharmaceutical composition) comprising such cells to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

For adoptive immunotherapy using antigen-specific T cells, cell doses in the range of about 10⁶-10¹⁰ (e.g., about 10⁹) are typically infused. Upon administration of the presently disclosed cells into the host and subsequent differentiation, T cells are induced that are specifically directed against the specific antigen. The modified cells can be administered by any method known in the art including, but not limited to, intravenous, subcutaneous, intranodal, intratumoral, intrathecal, intrapleural, intraperitoneal and directly to the thymus.

The presently disclosed subject matter provides methods for treating and/or preventing a neoplasia in a subject. The method can comprise administering an effective amount of the presently disclosed cells or a composition (e.g., a pharmaceutical composition) comprising such cells to a subject having a neoplasia.

Non-limiting examples of neoplasia include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, testicular cancer, anal cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Other carcinomas that may be treated with cells comprising the systems disclosed herein include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasia is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells and compositions comprising thereof can be used for treating and/or preventing blood cancers (e.g., leukemias, lymphomas, and myelomas) or ovarian cancer, which are not amenable to conventional therapeutic interventions. In certain embodiments, the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia. In certain embodiments, the lymphoma is Hodgkin's lymphoma or non-Hodgkin's lymphoma. In certain embodiments, the non-Hodgkin's lymphoma is B-cell non-Hodgkin's lymphoma or T-cell non-Hodgkin's lymphoma

The subjects can have an advanced form of disease, in which case the treatment objective can include mitigation or reversal of disease progression, and/or amelioration of side effects. The subjects can have a history of the condition, for which they have already been treated, in which case the therapeutic objective typically includes a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasia but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts and are suitably defined for each different neoplasia. Features typical of high-risk subgroups are those in which the tumor has invaded neighboring tissues, or who show involvement of lymph nodes.

Another group have a genetic predisposition to neoplasia but have not yet evidenced clinical signs of neoplasia. For instance, women testing positive for a genetic mutation associated with breast cancer, but still of childbearing age, can wish to receive one or more of the cells described herein in treatment prophylactically to prevent the occurrence of neoplasia until it is suitable to perform preventive surgery.

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed cells or a composition (e.g., a pharmaceutical composition) comprising such cells to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

The presently disclosed subject matter further provides methods for increasing an immune activity of an immunoresponsive cell. In certain embodiments, the method comprises introducing to the immunoresponsive cell a system disclosed herein to the immunoresponsive cell.

The presently disclosed subject matter provides methods for activating an antigen presenting cell (APC) in a subject. In certain embodiments, the method comprises administering to the subject an effective amount of the cells or a composition (e.g., a pharmaceutical composition) comprising such cells.

The presently disclosed subject matter provides methods for conditioning a subject for bone marrow transplant. In certain embodiments, the method comprises administering to the subject an effective amount of the cells or a composition (e.g., a pharmaceutical composition) comprising such cells.

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), and inducible Caspase 9 Suicide gene (iCasp-9). In certain embodiments, the cells include a truncated human epidermal growth factor receptor (EGFRt) polypeptide. The EGFRt polypeptide can enable T cell elimination by administering anti-EGFR monoclonal antibody (e.g., cetuximab). EGFRt can be covalently joined to the upstream of any polypeptide disclosed herein. The suicide gene can be included within the vector comprising nucleic acids encoding any polypeptide disclosed herein. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated T cells comprising any polypeptide or system disclosed herein. The incorporation of a suicide gene or EDFRt into the presently disclosed polypeptide or system gives an added level of safety with the ability to eliminate the majority of the engineered T cells within a very short time period. A presently disclosed cell (e.g., a T cell) incorporated with a suicide gene can be pre-emptively eliminated at a given timepoint post engineered T cell infusion or eradicated at the earliest signs of toxicity.

### 10. Examples

### 10.1 Materials and Methods

### 10.1.1 Vector and DNA Construct Design

Constructs were expressed from retroviruses or transposons. Tables 1-3 show the sequences for exemplary construct and construct elements described herein. Vector construct maps are shown in the figures. CAR and other receptor constructs used murine protein sequences except for Fas 4-1BB, which utilized the human 4-1BB costimulatory domain. DNA constructs were generated via standard molecular biology techniques including overlap extension PCR and restriction site-based cloning and ligated directly into pENTR1a no ccdB (Addgene 17398) or the MMLV-based retroviral vector LZRS-Rfa (Addgene 31601). SnapGene 6.2 (SnapGene) was used to design vectors. DNA templates were obtained as gBlocks or gene syntheses from Integrated DNA Technologies (IDT), or purchased from Origene, Addgene, and Sino Biological. pENTR1a-based constructs were transferred via Gateway cloning (Gateway LR Clonase II Enzyme mix, Invitrogen, 11791020) to LZRS-Rfa, piggybac transposons, or piggybac transposon ITR-flanked retroviral vectors containing attR recombination sites, designed in this study. These vectors were designed for stable high-level expression of retroviral vectors integrated into packaging lines using piggybac transposition. The vectors, named PB-MMLV-puro, PB-MPSV-puro, and PB-SIN-puro include the backbone and ITR and insulator domains of the piggybac transposon vector PB-EF1α-MCS-IRES-GFP (System Biosciences PB530A-2) and contain a 5′ compound SV40 enhancer with hybrid RSV-MMSV LTR (based on SERS11 design), leader sequence containing a modified MESV packaging signal (from MP71), attR sites flanking ccdB and chloramphenicol resistance genes, woodchuck hepatitis virus post-transcriptional regulatory element (WPRE), and followed by 3′ LTR regions from LZRS (MMLV), MP71 (MPSV), or inactivated MMLV LTR from pSIN (SIN). These vectors also contain an hPGK-promoter driving a Thy1.2-T2A-puroR selection cassette. PB-SFG5.3-BlastR contains the full 5′ LTR and packaging signal from SFG, attR sites flanking ccdB and chloramphenicol resistance genes, WPRE, 3′ SFG LTR, and hPGK-promoter driving a Thy1.2-T2A-BlastR selection cassette. The transposon PB-EF-1α-intron was generated by replacing the EF-1α core promoter in PB-EF1α-MCS-IRES-GFP with the full EF-1α intron-containing promoter. PB-EF-1α-intron-WPRE contains an added 3′ WPRE. gLuc-PD-1H-CD24-GPI-P2A-EGFP encodes a surface-expressed Gaussia luciferase with PD-1 hinge and membrane anchor based on mouse CD24 GPI transfer signal to localize BLI signal to gLuc-expressing cells and also encodes an EGFP reporter. CBR-P2A-hCD8-T2A-puroR is a construct containing click beetle red luciferase (CBR) along with a human CD8 surface reporter and encoding puromycin resistance. PB-SIN-puro BFP-SV40-NFAT-dEGFP, PB-SIN-puro BFP-SV40-AP-1-dEGFP, and PB-SIN-puro BFP-SV40-NFkB-dEGFP are transcription factor reporter vectors, and PB-EF-1α-intron hCD8-T2A-integrin-alpha-V and PB-EF-1α-intron mCD4-P2A-integrin-beta-3 encode components of human vitronectin receptor and hCD8 and mCD4 reporters, respectively.

### 10.1.2 Engineering a Leucine-Zipper Based Sorting System

To develop a platform to simplify purification of dual-vector-transduced cells by enabling single-step magnetic-bead cell sorting system, a heterodimerizing leucine zipper pair encoded by two vectors was utilized: (1) a secreted affinity-tagged zipper and (2), a membrane-bound capture-zipper designed to pair uniquely in double-transduced cells. This leucine zipper pair, termed RR12EE345L and EE12RR345L, was engineered for high-affinity heterodimerization via charge-based attraction of arginine (R) and glutamate (E) residues (Table 1).

### 10.1.3 Retrovirus Production

Retroviruses were produced in Phoenix-Eco or Phoenix-Eco alpha-V beta-3, designed to enhance adhesion of Phoenix-Eco to culture flasks. Phoenix-Eco or Phoenix-Eco alpha-V beta-3 were transfected with LZRS-vector constructs using Effectene® transfection reagent (Qiagen, 301425) as follows: complexes were generated using 2 pg vector DNA in 16 μL of enhancer, 20 μL of Effectene® and used to transfect Phoenix cells plated on a 10 cm dish at 2.5×10⁶ cells one day prior to transfection. On the day following transfection, cells were removed from dishes and assessed by flow cytometry for construct expression and selected in puromycin 2 μg/mL (Santa Cruz Biotech, sc-108071B). Phoenix-Eco or Phoenix-Eco alpha-V beta-3 were transfected with PB-vector constructs as above, with addition of 1 μg of the hyperactive piggyBac transposase vector pCMV-hyPBase⁷⁰ (Sanger Institute, UK) for stable integration. Transfected cells were selected one day after transfection with puromycin 2 μg/mL (Santa Cruz Biotech, sc-108071B) or blasticidin 10 μg/mL (Santa Cruz Biotechnology, sc-204655). Retroviral supernatant was collected from fully selected stable packaging lines grown from T175 flasks. Supernatants were filtered through 50 mL syringes fitted with Millex-HV Syringe Filters (Millipore, 0.45 μm, PVDF, SLHVR33RS) and polyethylene glycol solution concentrate was added (5× concentrate: PEG 8000 MW Promega, V3011, 40% weight/volume containing 2.4% NaCl weight/volume), and virus was precipitated over 1-2 days at 4 C. Precipitated virus was centrifuged at 3000×g for 15 minutes at 4 C and pellets were resuspended in 500 μL of T cell media and frozen at −80 C or used directly.

### 10.1.4 Retroviral Transduction

Tumor cell lines were transduced with retroviral supernatant containing polybrene 8 μg/mL (Sigma, H9268-10G) in 24-well plates by spinfection at 1500 RPM at 32 C for 1 hour. Cells were transferred to T25 flasks on the following day for expansion. For primary mouse T cell transduction, splenic T cells were enriched by negative selection of splenocytes with anti-CD19 microbeads to deplete B cells (Miltenyi, 130-121-301) and were stimulated on plate-bound anti-CD3/CD28 for 1 day (2 pg/mL each, clones 145-2C11 and 37.51, respectively, BioXCell). Activated T cells were transduced on day 1 after stimulation using combinations of PEG-precipitated retroviral concentrates encoding different constructs adsorbed onto non-tissue culture treated 6-well plates coated with anti-CD3/CD28 2 μg/mL each and retronectin 20 μg/mL (Takara, T100B) at 1-2×10⁶ cells/well. On day 2, T cells were either re-transduced, or transferred to 6-well plates coated with anti-CD3/CD28. Cells were removed on day 3, Zip-sorted, and expanded in 50 IU/mL rhIL-2 (Proleukin).

### 10.1.5 Target Cell Line Construction

BM185-CD19 was constructed by transducing BM185 cells with LZRS ffluc-Thy1.1-Neo and CD38 was deleted for use in concurrent studies using Cas9-NLS (UC Berkeley MacroLab), CD38 gRNA UAAAUUCAUAGUUAGCCAUU (SEQ ID NO: 123, Synthego), and Lonza SF buffer kit (Lonza, V4XC-2032) with a 4D Nucleofector using code DN100. Subclones were identified that were CD19⁺ CD38⁻. BM185-CD20 was similarly generated as a clone uniformly expressing the LZRS CD20 and LZRS ffluc-Thy1.1-Neo transgenes. CD19 was deleted with CD19 gRNA UGAUUCAAACUGCUCCCCCG (SEQ ID NO: 124) and CD38 deleted with the CD38 gRNA, after which cells were negatively selected for CD19 (CD19 microbeads, Miltenyi, 130-121-301) and CD38 (anti-CD38 PE, anti-PE microbeads, Miltenyi, 130-048-801). BM185-CD19-CD20 expresses LZRS ffluc-Thy1.1-Neo. BM185-CD19-FasL was generated from a BM185-CD19 ffluc-Thy1.1-Neo clone generated for concurrent studies with deletion of CD38 and CD22 (CD22 gRNA UGUCAUUGGCACGUAUCGGG, SEQ ID NO: 125, Synthego) and transduced with LZRS FasL and subcloned. BM185-CD20-PD-L1 and BM185-CD20-CD200 were similarly generated from CD38, CD22-deleted clones modified to express PD-L1 (LZRS PD-L1) or CD200 (PB-EF-1α-intron CD200R1, pCMV-hyPBase, SF buffer kit V4XC-2032, Lonza 4D Nucleofector, code DN100). Transposition reactions used 1-2 pg of vector DNA and 0.5-1.0 pg of pCMV-hyPBase transposase DNA. These cell lines were then subcloned. C1498-CD19 and C1498-CD20 versions were generated by transduction with SFG CD19 and LZRS CD20 and LZRS ffluc-Thy1.1-Neo and immunomagnetically sorted for high transgene expression. However, long-term retroviral expression in C1498 was unstable, so a stable C1498 CBR-hCD8-puro clone was generated using PB-EF-1α-intron transposon and generated subcloned versions individually transposed with the PB-EF-1α-intron transposon vectors encoding CD19, hCD19, CD20, CD79bΔ, or BAFF-R (transposon, pCMV-hyPBase, SF buffer kit V4XC-2032, Lonza 4D Nucleofector, code DA100). CD79bΔ refers to a chimeric protein comprising mouse CD79b extracellular domain with a CD28TM and truncated CD3ζΔ to promote surface expression in the absence of CD79a. For Incucyte® live-cell microscopy analysis, target cells were transduced with PB-MSPV-puro iRFP713-P2A-hygro-E2A-TAA-WPRE and selected in hygromycin B (Santa Cruz Biotechnology, sc-29067) at 0.8 mg/mL to enable near-infrared imaging.

### 10.1.6 Zip-Sorting

Transduced C1498 or T cells were incubated with anti-FLAG (Miltenyi, 130-101-591) or anti-CD34 (Miltenyi, 130-046-702) beads at 30 μL of beads per 10⁷ cells for 30 minutes at 4 C in PBS 2 mM EDTA+0.5% BSA, washed in PBS 2 mM EDTA+0.5% BSA, centrifuged 1200 RPM×5 minutes and resuspended in 500 pL PBS 2 mM EDTA+0.5% BSA. Cells were sorted on LS columns (Miltenyi, 130-042-401) by washing 3× (1 mL, 2 mL, 3 mL) with PBS 2 mM EDTA+0.5% BSA and eluting with 5 mL T cell media. Zip-sorted T cells were used for in vitro experiments on days 4-6 post-stimulation and injected into mice for in vivo experiments on day 5 post-stimulation. Sort yield is defined as 100*(dual-transduced cells recovered/dual-transduced cells present pre-sort).

### 10.1.7 Incucyte® Imaging

T cells were transduced with either PB-SFG5.3-BlastR EGFP (constitutive EGFP) or NFKB reporter vectors (inducible, destabilized EGFP reporter), added at 2×10⁴ cells/well to 96-well flat-bottom plates, and co-cultured with iRFP713-expressing target lines at varying effector:target (E:T) ratios in T cell media without rhIL-2. T cell and tumor cell line fluorescence was imaged simultaneously every 3 hours with 10× objective, 4 images per well, in an Incucyte® SX5 (Sartorius). For stress-test experiments, targets were added back to the plate at 1:1 initial E:T ratio at 24 and 48 hours after initial assay setup. Data were analyzed using Incucyte® Software v2021A.

### 10.1.8 Continuous Live-Cell Microscopy

T cells were transduced with either PB-SFG5.3-BlastR EGFP (constitutive EGFP) or NFAT, AP-1, or NFKB reporter vectors (inducible, destabilized EGFP reporter), added at 2×10⁴ cells/well to 96-well flat-bottom plates, and co-cultured with iRFP713-expressing target lines at varying E:T ratios in T cell media without rhIL-2. T cell and tumor cell line fluorescence was imaged simultaneously every 3 hours with 10× objective, 4 images per well, in an Incucyte® SX5 (Sartorius). For stress-test experiments, targets were added back to the plate at 1:1 initial E:T ratio at 24 and 48 hours after initial assay setup. For T cell cluster analysis, green fluorescence object threshold was set to minimum of 5000 μm², edge split off, eccentricity maximum 1.0, hole fill 0 μm². Data were analyzed using Incucyte® Software v2021A.

### 10.1.9 Bioluminescence-Based Target Lysis Assay

T cells were incubated in triplicate in 96-well U bottom plates for 24 hours at varying E:T ratios with 0.5-1×10⁴ luciferase-expressing targets in T cell media lacking rhIL-2. A no-T cell row was added to obtain relative maximum luciferase activity. For experimental readout, luciferin (Gold Bio LUCK-2G) was added to wells to achieve final 200 μg/mL concentration and luciferase activity was analyzed on a Tecan SPARK luminometer/fluorimeter (Tecan). Target relative percent viable values were calculated as 100*(experimental well activity units/maximum activity units).

### 10.1.10 Flow Cytometry

Flow cytometry analysis acquisition was performed on an LSR-II or FACSymphony™ X50 using FACSDiva™ software (BD Biosciences). Analysis was performed using FlowJo software (BD Biosciences, version 10.8.1). Cell viability was assessed with DAPI (Calbiochem, 5087410001). MFI refers to geometric mean fluorescence intensity. Intracellular flow cytometry analysis was performed by first antibody staining cells for surface markers and live/dead status using LIVE/DEAD Fixable Blue Dead Cell Stain Kit (Invitrogen, L23105), followed by permeabilization using the Foxp3/Transcription Factor Staining Buffer Set (Invitrogen, 00-5523-00) and antibody stained for intracellular contents. CAR expression was detected by flow cytometry with antibodies against affinity tags including Myc, Streptavidin tag, FLAG, hCD20 mimotope (Rituximab-APC), and the hCD34 tandem epitope with their respective antibodies. Streptavidin tag was also detected using Streptactin-PE for some experiments (Iba Biosciences, 6-5000-001). Rituximab was obtained from the MSKCC pharmacy and APC conjugated (APC Conjugation Kit—Lightning-Link, Abcam, ab201807). Anti-streptavidin tag purified antibody (Genscript) was also APC conjugated. The BAFF-R CAR was stained with 1 μg of hBAFF-R hFc (Sino Biological, 16079-H02H), followed by anti-hFc antibody. Similarly, PD-1-DNR interaction with PD-L1 was assessed by staining with 1 μg Recombinant Mouse PD-L1/B7-H1 Fc Chimera Protein (R&D Systems, 1019-B7-100), followed by anti-hFc antibody.

### 10.1.11 Complement Lysis Assay

T cells were incubated at 5×10⁴ cells/well in 96-well U bottom plates with rabbit complement (final concentration 10%, Cedarlane, CL3051)±100 μg/mL anti-Thy1.1 (clone 9E12, BioXCell) for 30 minutes at 37 C. Subsequently, viable cells were enumerated by flow cytometry, measuring DAPI-negative viable cells using CountBright™ Beads (Invitrogen, C36950). Relative T cell survival was calculated as 100*(viable cells: antibody+complement/viable cells: complement only).

10.1.12 iCaspase9 Activation Assay

For dimerizer titration, T cells were incubated at 3×10⁴ cells/well in 96-well U bottom plates for 24 hours in T cell media containing 50 IU/mL rhIL-2 and varying concentrations of AP20187 (B/B homodimerizer, Takara, 635058). After 24 hours, cells were analyzed for DAPI-negative viable cells using CountBright™ beads (Invitrogen, C36950). Relative T cell survival was calculated as 100*(viable cells: dimerizer/viable cells: DMSO).

### 10.1.13 Reactive Oxygen Species Assessment

Unstimulated day 5 T cells were plated at 5×10⁴ cells/well in 96-well U bottom plates, washed once with 200 μL of PBS, resuspended in 100 μL of PBS containing 1 M CM-H₂-DCFDA (Invitrogen, C6827) or 5 M MitoSOX™ Red (Invitrogen, M36008) for 30 minutes at 37° C. Cells were washed twice in 200 μL of PBS+0.5% BSA, stained with anti-CD4/CD8, and analyzed by flow cytometry for DAPI-negative viable cells.

### 10.1.14 Transcription Factor Reporter Assay

T cells were transduced with transcription factor reporter vectors on day 1 post-stimulation (to ensure equivalent reporter transduction among T cells subsequently transduced with different constructs) and on day 2 the cells were transduced with different CAR construct vectors. T cells were subsequently Zip-sorted or left unsorted for further analysis. T cells were assessed for EGFP induction by flow cytometry following culture with or without target cells in BFP-transduction reporter-positive cells.

### 101.1.15 In Vivo Experiments

Female BALB/cJ (Jackson Laboratory, 00651) and B6(Cg)-Tyr^{c-2J}/J albino B6 (Jackson Laboratory, 000058) mice were used in experiments at 7-12 weeks of age. Albino B6 were utilized to enhance BLI sensitivity given absence of fur pigmentation. CD45.1 congenic BALB/c mice (Jackson Laboratory, 006584) were bred at MSKCC. Animal studies were conducted in the MSKCC vivarium under a protocol approved by the MSKCC Institutional Animal Care and Use Committee. Mice were evaluated at least twice daily and euthanized when reaching any of the following humane endpoints: weight loss>25%, labored breathing, moribund status, hind-limb paralysis, development of ascites, tumor>2 cm or interfering with bodily functions.

### 10.1.16 BM185 Pre-B Acute Lymphoblastic Leukemia Mouse Model

BALBc/J mice were sublethally irradiated with 450 cGy of gamma radiation (Gammacell, cesium source), rested for 4 hours, and then injected with varying doses of BM185 cell lines via tail vein in 200 μL of DMEM without additives (day 0). Mice were randomized into groups following leukemia injection. On day 2, Zip-sorted T cells were injected into the retroorbital plexus in 150 μL of DMEM. T cell and BM185 cell doses are depicted in figures above survival or BLI plots. Mice were evaluated daily for evidence of reaching humane endpoints described in the In vivo experiments section. Mice were serially assessed for leukemia progression via firefly luciferase (ffluc)-based BLI. In some experiments, spleens or bone marrow (BM) were obtained at the time of euthanasia for further analysis. Spleens and BM were dissociated through 40-micron filters, red blood cell lysed (Hybri-Max™, Sigma, R7757), and stained with anti-CD38, Thy1.1, CD19, and CD20. Spleens or BM with <0.1% Thy1.1⁺CD3⁻ BM185 cells or <10 events were excluded from analysis of BM185 surface phenotype. In some experiments, mice were bled via retroorbital plexus, blood was red blood cell-lysed, and stained for flow cytometry analysis.

### 10.1.17 C1498 Acute Myeloid Leukemia Mouse Model

Albino B6 mice were sublethally irradiated with 550 cGy of gamma radiation (Gammacell, cesium source), rested for 4 hours, and then injected with varying doses of C1498 cell lines via tail vein in 200 μL of DMEM without additives (day 0). Mice were randomized into groups following leukemia injection. On day 2, Zip-sorted T cells were injected into the retroorbital plexus in 150 μL of DMEM. T cell and C1498 cell doses are depicted in figures above survival or BLI plots. Mice were evaluated daily for evidence of reaching humane endpoints described in the In vivo experiments section. Mice were serially assessed for leukemia progression via CBR luciferase based BLI. In some experiments, BM was obtained at the time of euthanasia for further analysis. BM was dissociated through 40-micron filters, red blood cell lysed (Hybri-Max, Sigma, R7757), and stained for hCD8 and tumor target antigens. BM with <0.1% hCD8+C1498 cells or <10 events was excluded from C1498 surface phenotype analysis. In BM of some CAR T cell treated mice, an amorphous debris was observed that simultaneously stained positively for all flow markers: hCD8, CD19, CD20, CD79b, and BAFF-R. This population was gated out of analyses. The C1498 model was less predictable than BM185, with mice sometimes dying overnight despite looking otherwise healthy on the prior night. Additionally, a subset of CAR T cell treated mice apparently cleared leukemia from the BM with extramedullary progression. Therefore, the number of available BM samples with C1498 to assess for antigen-loss escape was diminished compared with the BM185 model.

### 10.1.18 Bioluminescence Imaging (BLI)

For serial quantitative assessment of leukemia, mice were injected with D-luciferin (Gold Bio, LUCK-2G) at 150 mg/kg dose intraperitoneally. Ten minutes after injection, isoflurane-anesthetized mice were imaged using an IVIS Spectrum CT imaging system (PerkinElmer). For serial quantitative assessment of T cells, mice were injected with 100 g of water-soluble Coelenterazine (NanoLight Technology, 3031) into the retroorbital plexus and imaged immediately. Mice were imaged individually following injection.

### 10.1.19 Statistical Analysis

For BLI curves, groups were compared using area under the curve (AUC) analysis performed on log-transformed BLI values using a Vardi test⁷¹ with the function aucVardiTest in the R package clinfun. False discovery rate (FDR) correction was applied to account for multiple comparisons. Pairwise log-rank tests were performed by the function pairwise_survdiff in the R package survminer followed by FDR correction for multiple tests. All other statistical tests were performed using GraphPad Prism, with test type described in figure legends.

### 10.2 Results

### 10.2.1 Leucine Zipper Sorting System Facilitates Production of T Cells Expressing Multiple Cars and Switch Receptors.

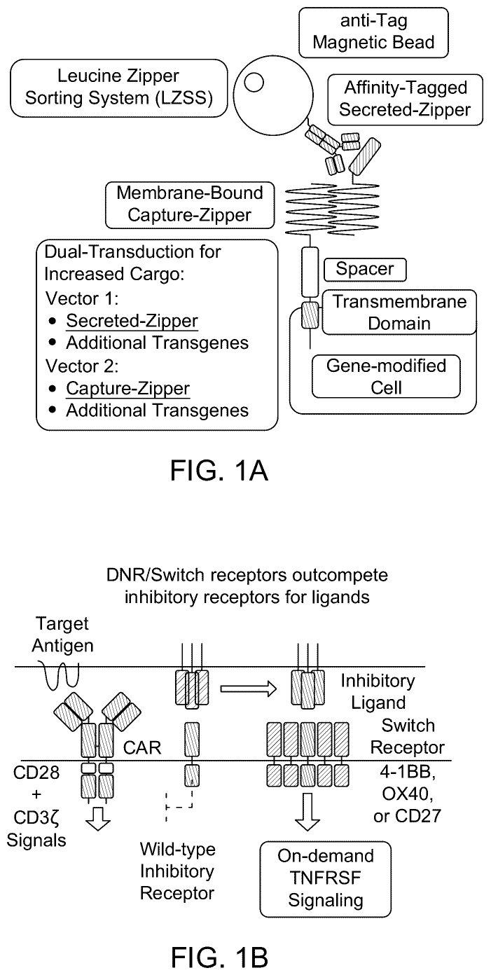

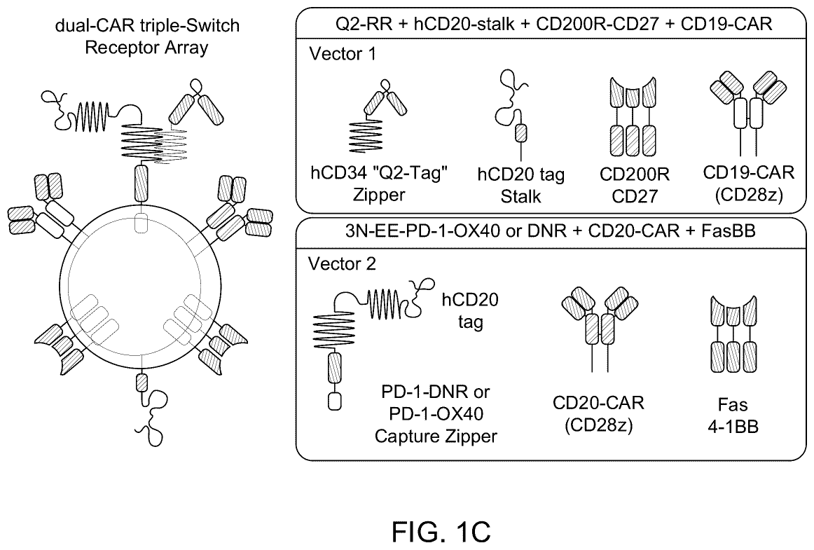

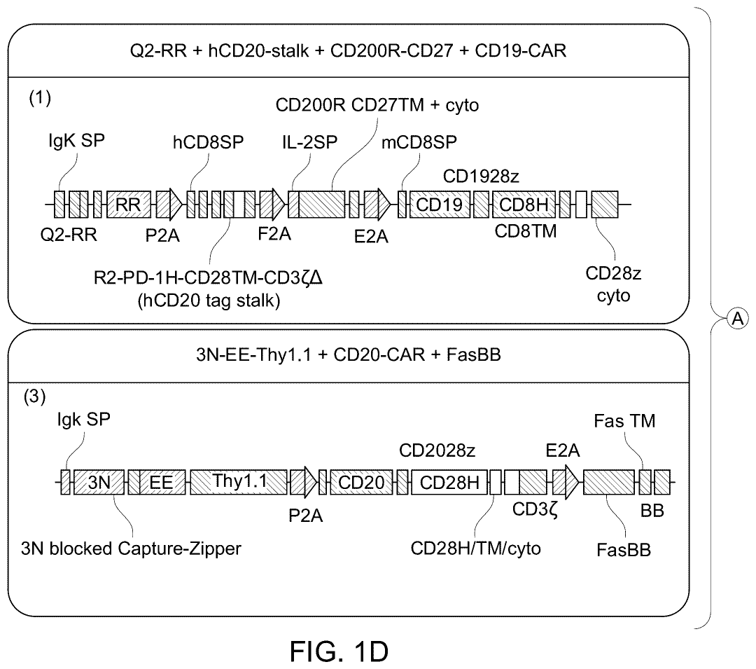

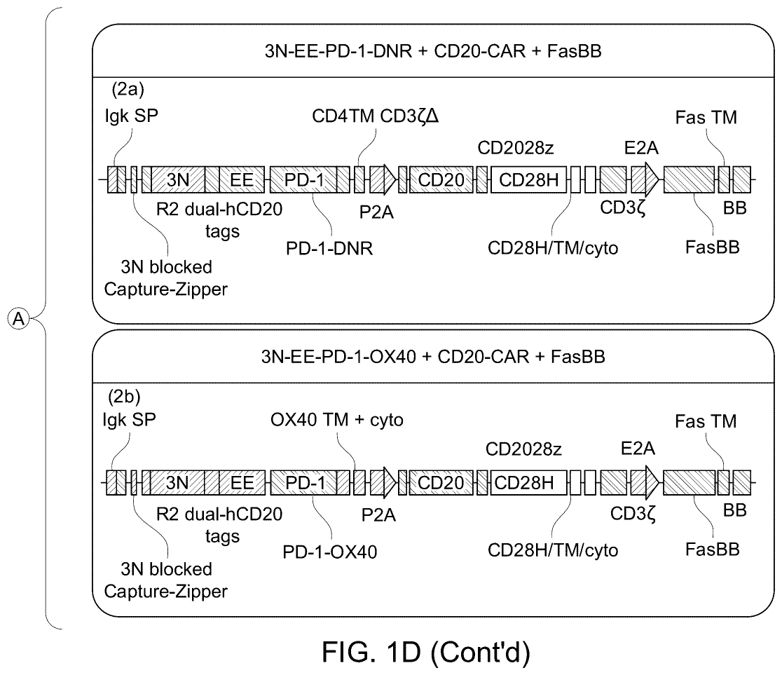

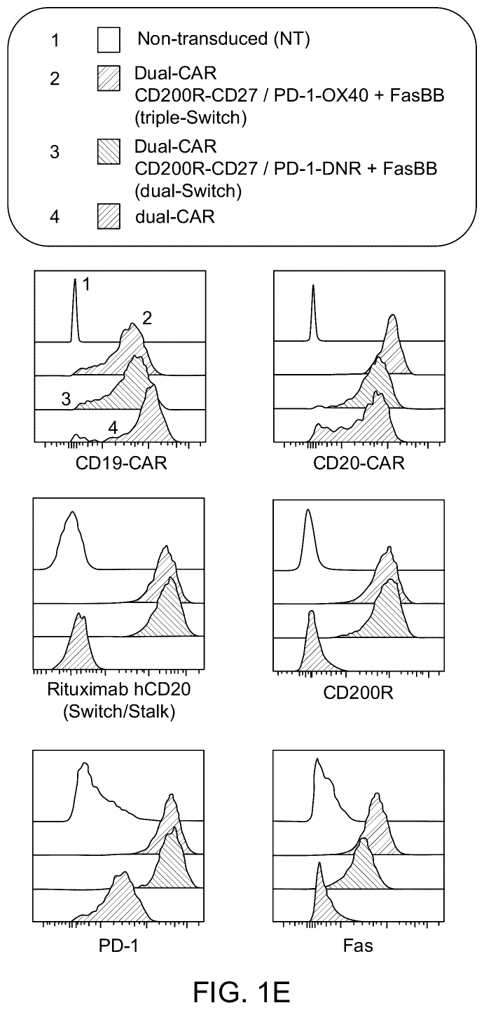

Switch receptors convert inhibitory ligand signals into positive costimulatory signals while simultaneously blocking interactions of these ligands with their native receptors (FIG. 1C). For example, a dual-CAR and multi-Switch CAR T cell can be generated by transducing a T cell with two vectors—vector 1 encoding a secreted leucine zipper having a tandem-CD34 tag (Q2 tag), a Rituximab® binding safety switch stalk, CD200R1-CD27 switch receptor (CD200R1-CD27), and a CD19-CAR; and vector 2 encoding a PD-1-OX40 (or PD-1-Dominant Negative Receptor) switch receptor having a 3N blocked capture-zipper, a Rituximab® binding cyclic CD20 mimotope tag, a CD20-CAR, and Fas-4-1BB switch receptor (FasBB). Vector maps for multi-Switch receptors are shown in FIG. 1D. Following transduction with the 2 vectors, primary T cells were zip sorted using anti-CD34 magnetic beads. The cells were then assessed for expression of the CARs, Rituximab® binding, CD200R1, PD-1 and Fas. FACS analysis high expression of CARs and switch receptors (FIG. 1E).

10.2.2 Capture-Zipper Modification does not Impair Interaction of PD-1 Dominant Negative Receptor with PD-L1.

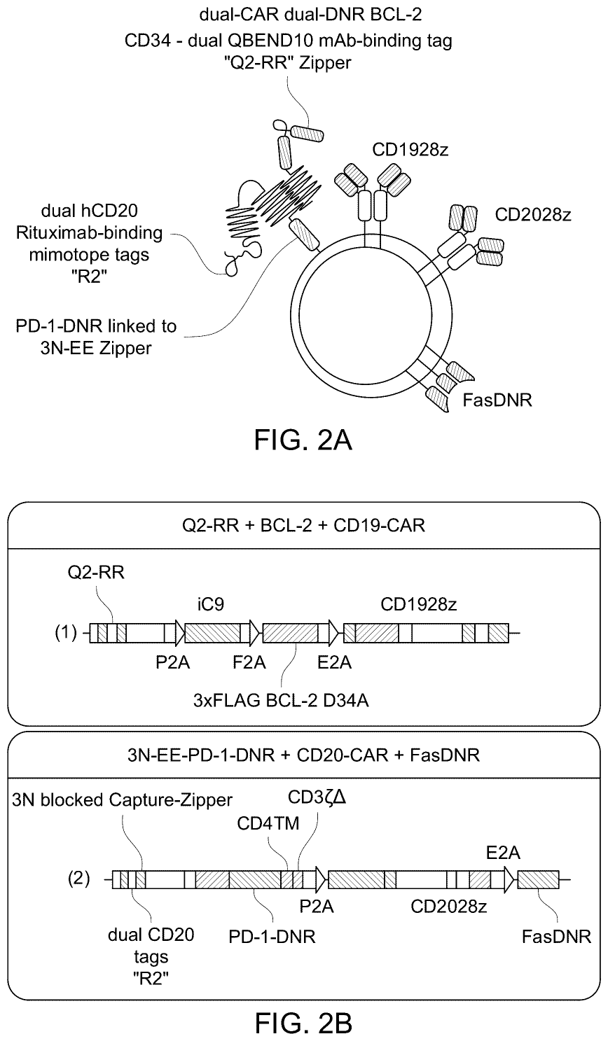

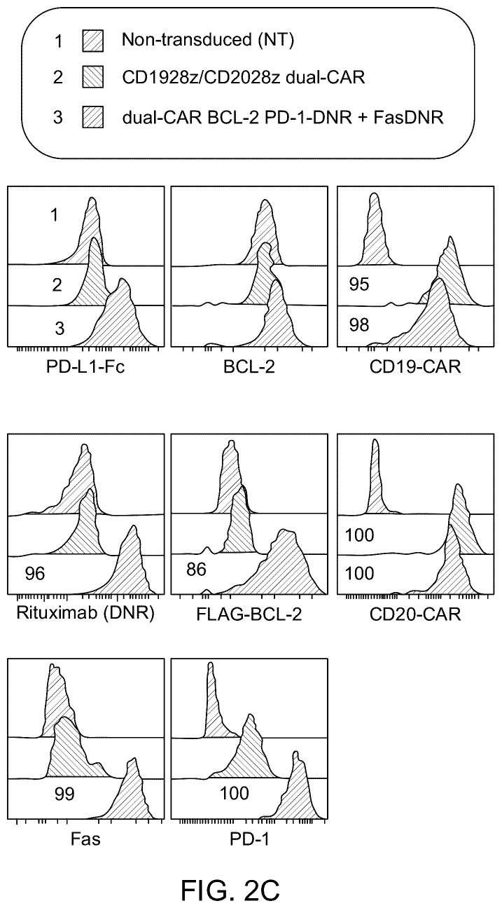

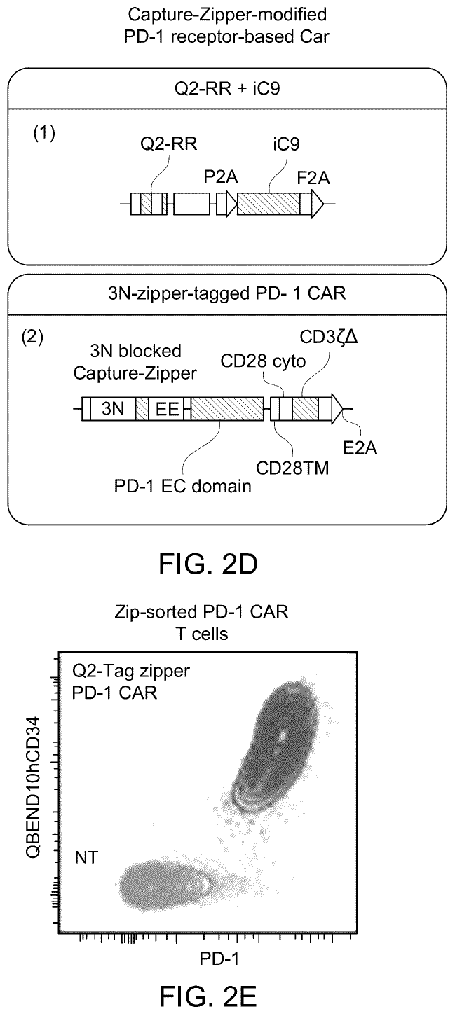

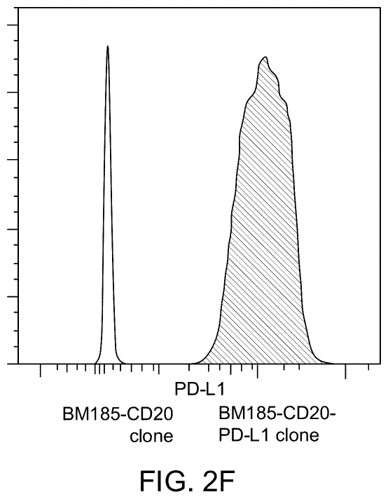

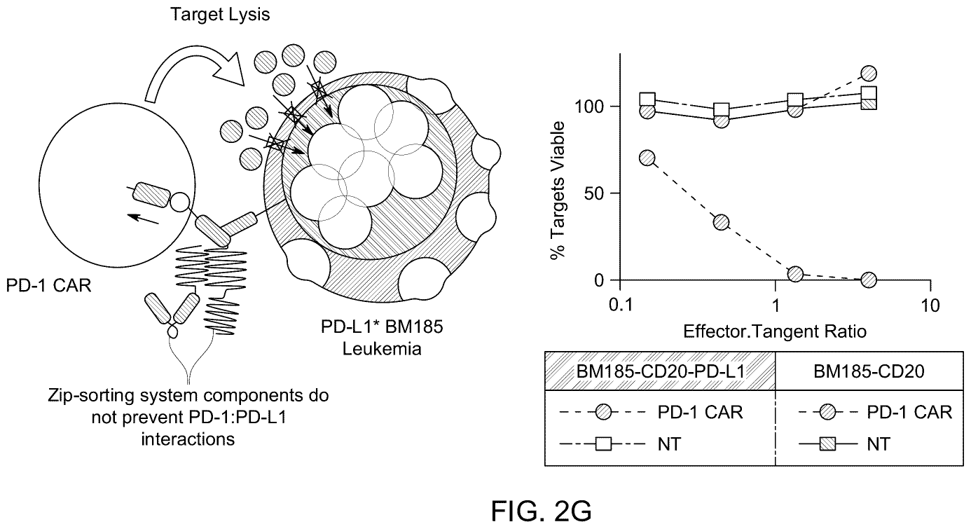

To test whether attachment of PD-1 to the capture zipper impedes PD-1 & PD-L1 interactions, a T cell expressing dual CARs (CD19, CD20), two dominant negative receptors (DNR) PD-1-DNR and Fas-DNR, and overexpressing caspase-cleavage-resistant Bcl2 was generated (FIGS. 2A, 2B). Dual transduced cells were purified by single-step Zip-sorting to obtain T cells showing high expression of all receptors (FIG. 2C). PD-L1 Fc was found to bind 3N capture-zipper modified PD-1 molecule, demonstrating absence of steric hindrance of ligand binding resulting from the capture-zipper (FIG. 2D-2F). Target cell viability studies revealed the efficacy of 3N blocked capture-zipper-based CAR cells in enabling selective sorting of dual-transduced T cells and killing of PD-L1⁺ leukemia cells (FIG. 2G).

### 10.2.3 Multi-Switch Receptor-Expressing CAR T Cells Demonstrate Enhanced NFkB Activity and Expansion Following Target Encounter.

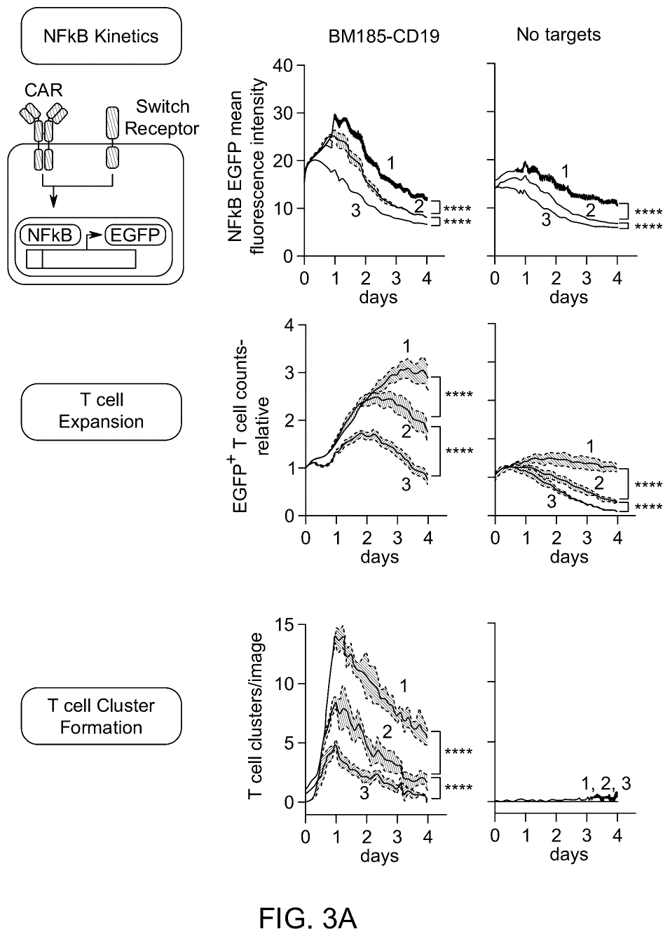

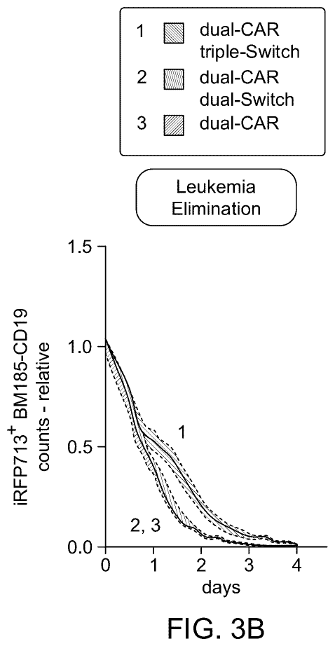

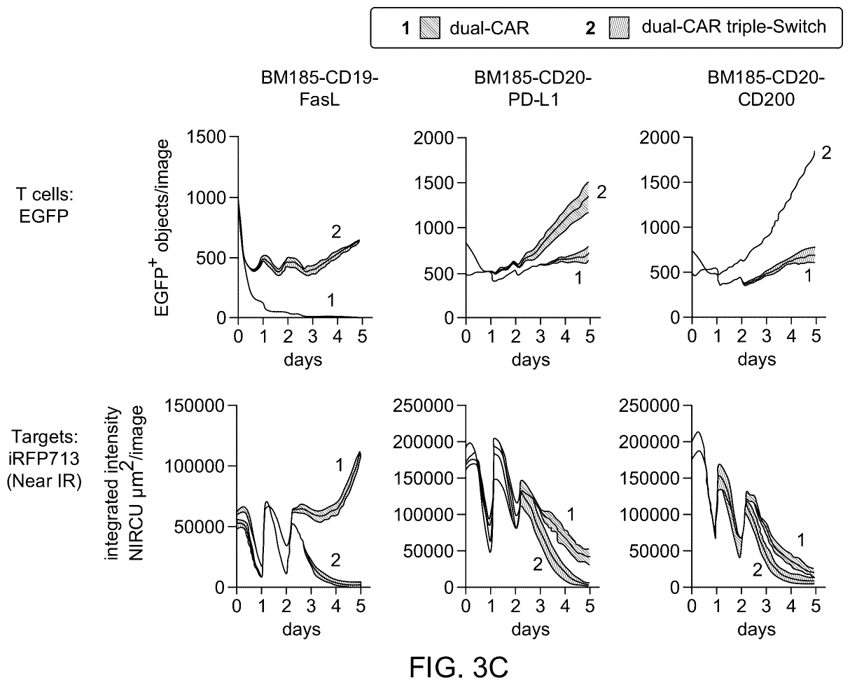

The effects of switch receptor expression on CAR T cell activity was assessed using live cell NFkB assay. FIG. 3A shows target cell dependent enhancement of NFkB reporter intensity that correlated with enhanced T cell expansion and T cell cluster formation in dual CAR triple switch and dual CAR dual switch expressing T cells compared to dual CAR T cells. Multi-Switch receptor-expressing CAR T cells showed similar potency as CAR T cells in eliminating leukemia cell target (FIG. 3B). Additionally, dual CAR triple switch expressing T cells were protected from FasL expression on target cells and proliferated to a greater extent following interaction with leukemia cells expressing PD-L1 and CD200 inhibitory ligands.

### 10.2.4 Expression of Multiple Switch Receptors Inhibits Surface Expression of Inhibitory Ligands.

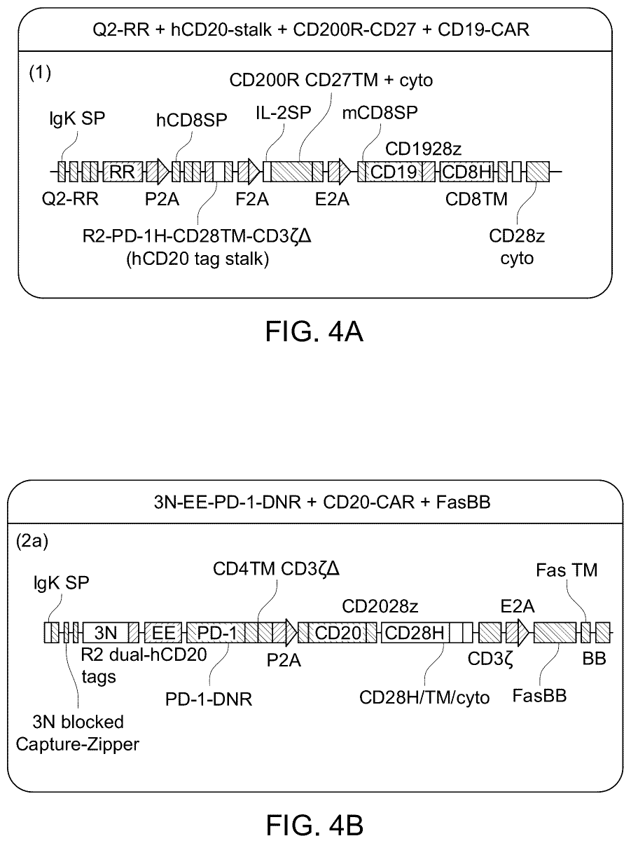

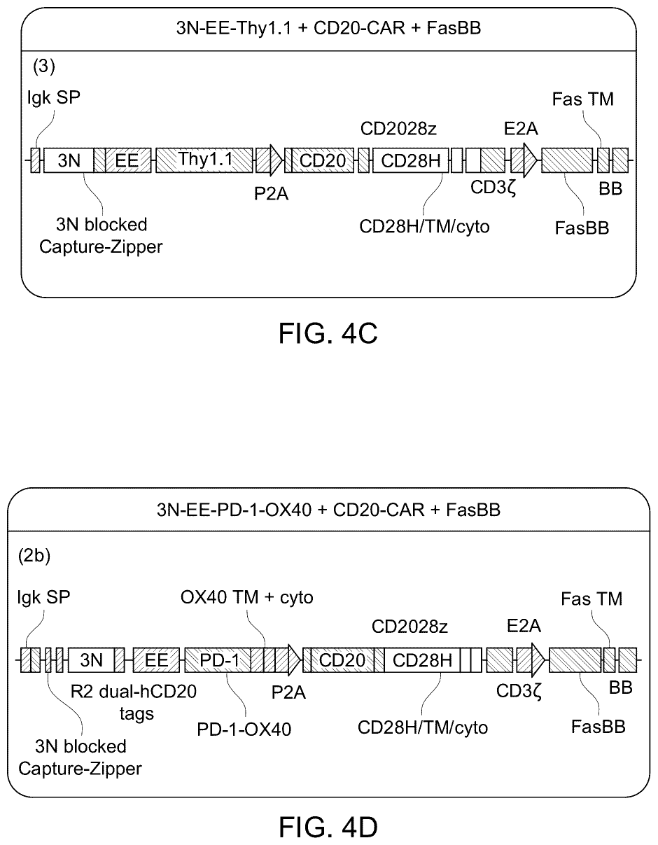

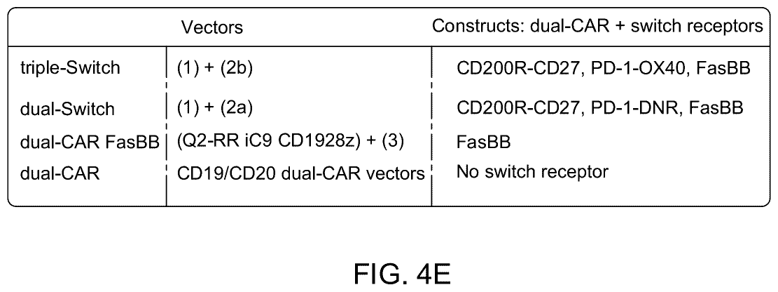

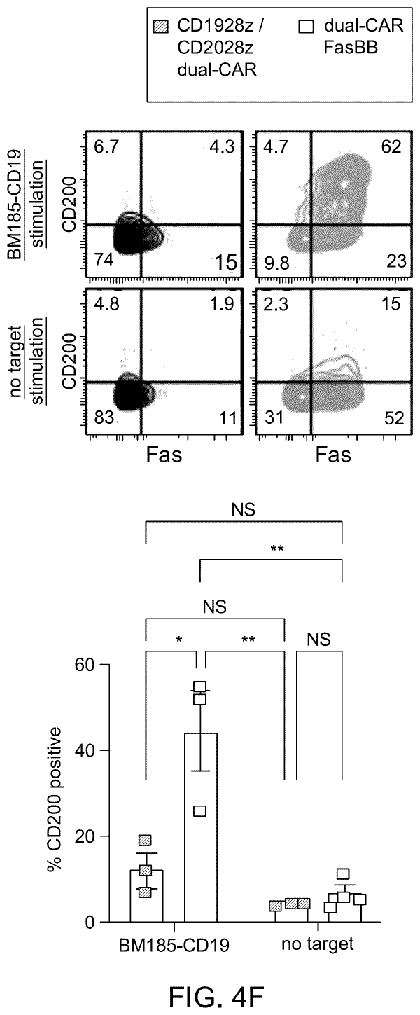

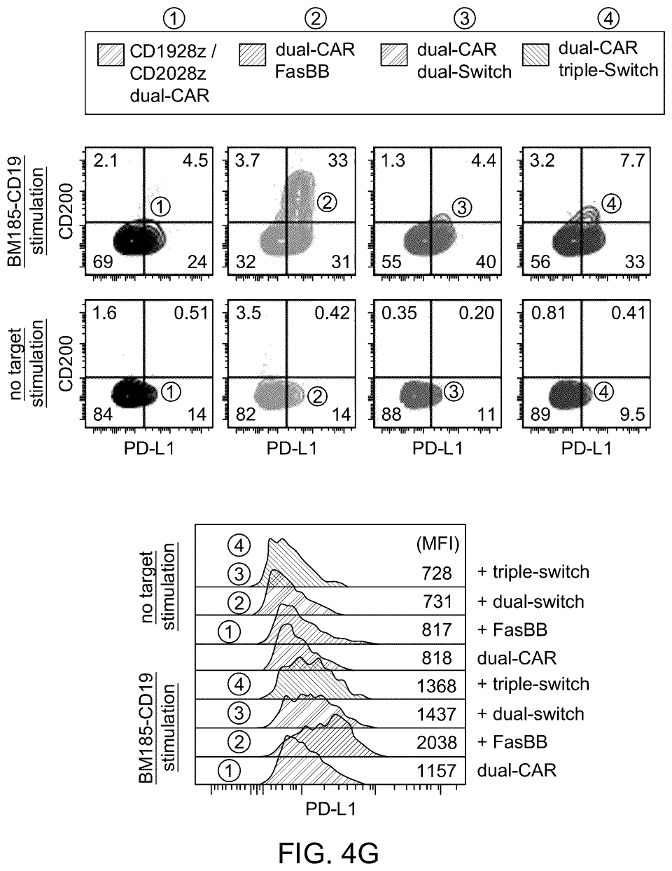

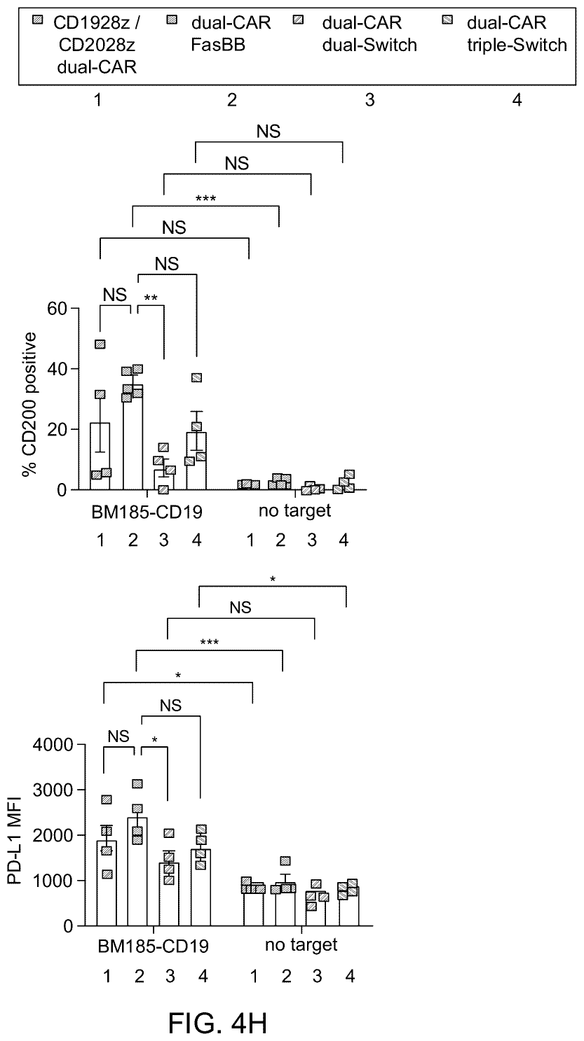

CD19/CD20 dual CAR T cells expressing Fas-4-1BB switch receptor (FasBB) were challenged with CD 19 expressing BM185 leukemia target cells. FACS analysis revealed a target cell dependent upregulation of CD200 in the dual CAR/FasBB T cells (FIG. 4F). Co-expression of PD-1-OX40 and CD200R1-CD27 switch receptors in dual CAR/FasBB T cells (dual CAR, triple switch T cells) inhibited surface expression of inhibitory ligands PD-L1 and CD200 (FIGS. 4G, 4H).

### 10.2.5 Dual-CAR Multi-Switch Receptor T Cells Mediate Enhanced Clearance Of Leukemia.

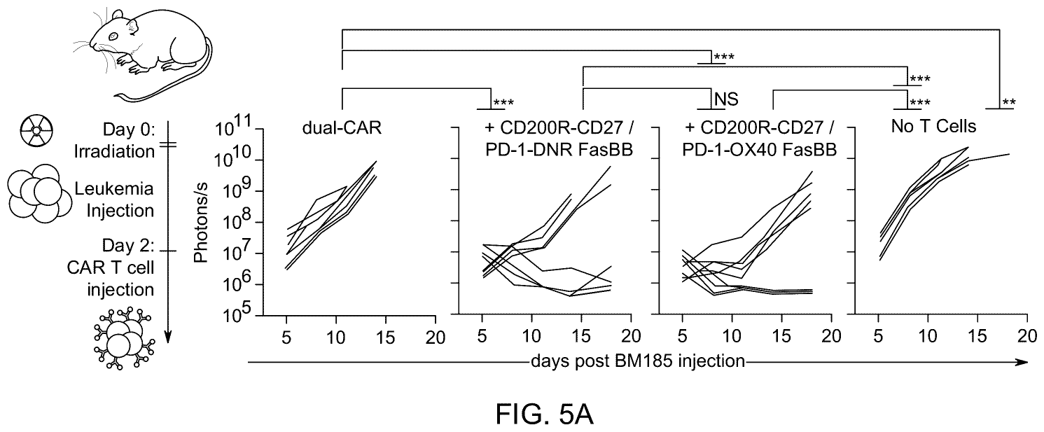

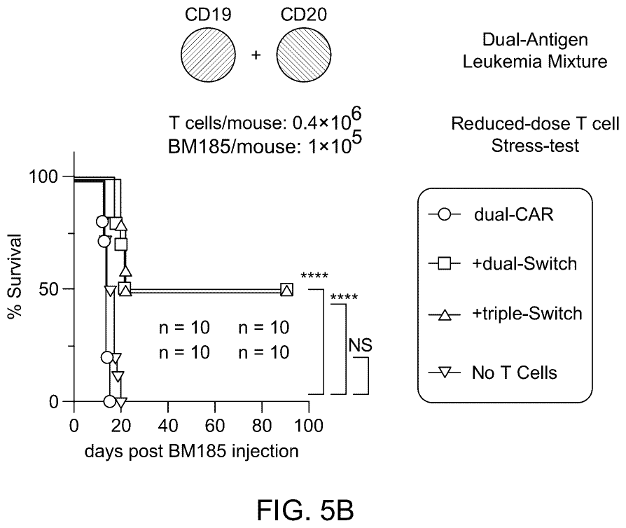

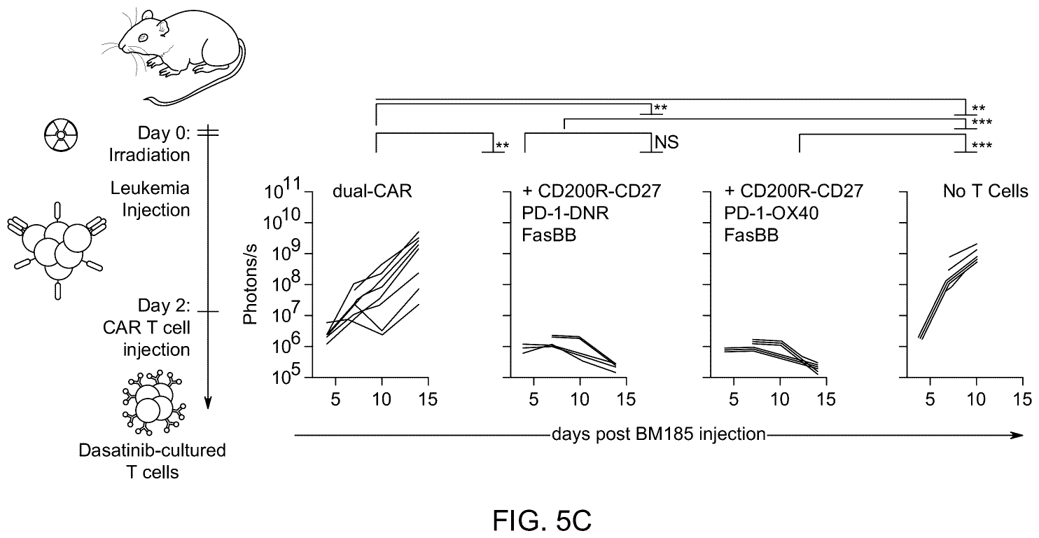

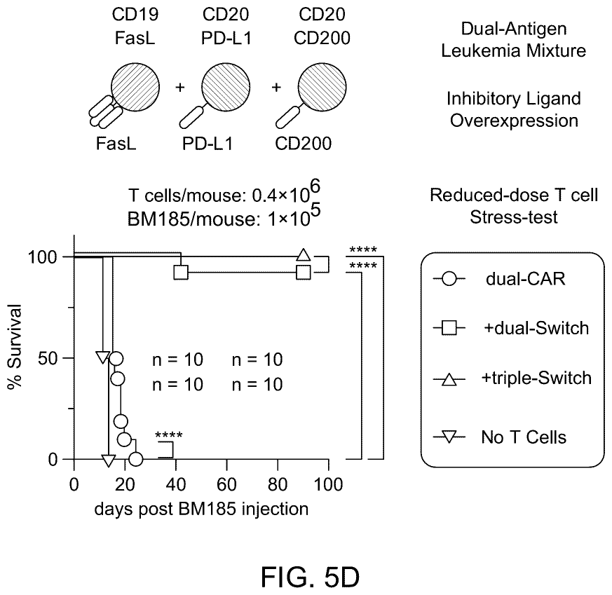

To test the effects of switch receptor expression on the ability of CAR T cells to eliminate cancer cell in vivo, BALB/c mice were injected with a mixture of CD19-BM185 and CD20-BM185 leukemia cells (luciferase expressing) in a 1:1 ratio (antigen-loss escape model), followed by treatment with a stress-test reduced dose of CD19/CD20 dual CAR T cells or CD19/CD20 dual CAR T cells expressing dual switch (CD200R1-CD27, FasBB, PD-1 DNR) or triple switch (CD200R1-CD27, FasBB, PD-1-OX40) receptors. Bioluminescence imaging (BLI) of the animals demonstrated clearance of leukemia in a subset of the tested mice (FIG. 5A), that correlated with increased survival as compared to dual CAR T cells having no switch receptor expression (FIG. 5B). These positive effects on leukemia cell elimination (FIG. 5C) and survival (FIG. 5D) were significantly enhanced in a higher subset of the animals when the T cells were cultured with the Src kinase inhibitor, dasatinib before administration to the leukemia bearing animals. This demonstrates that co-expression of multiple switch receptors can enhance the in vivo activity of dual-CAR T cells.

### 10.2.6 Addition of Dual-Switch Receptor Configuration to Triple CAR T Cells Enhances T Cell Expansion and Leukemia Clearance.

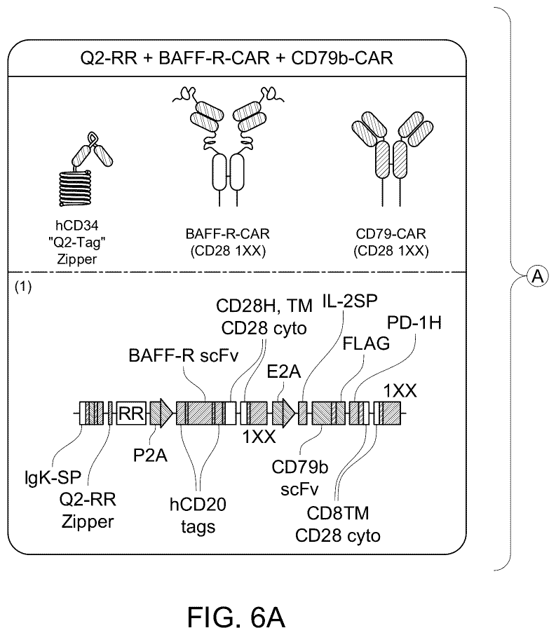

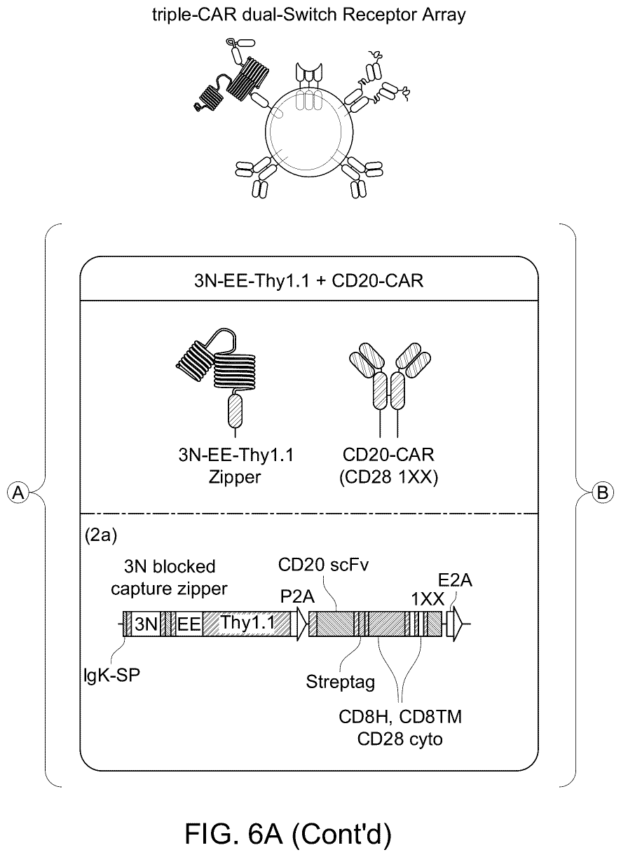

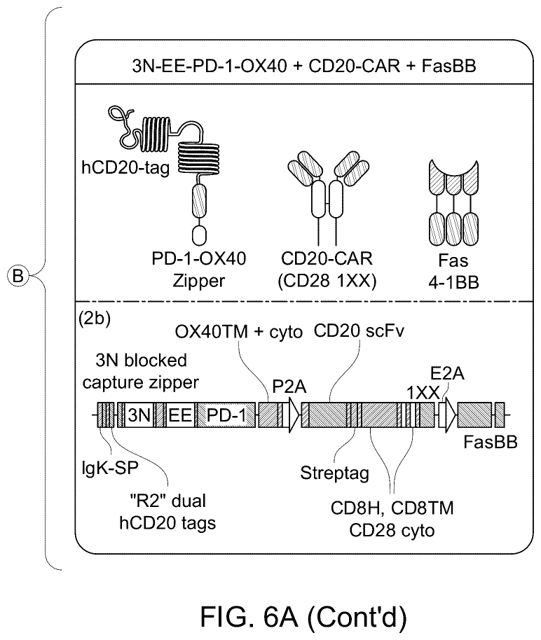

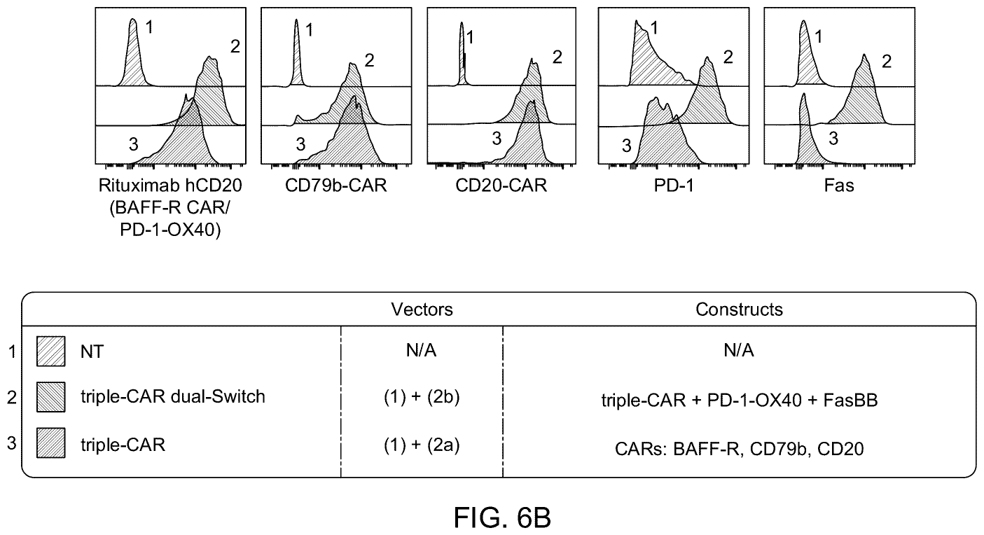

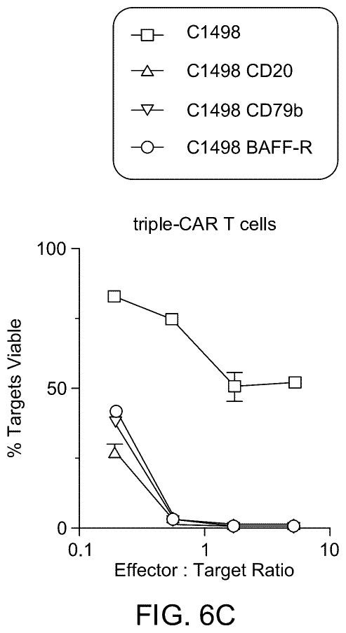

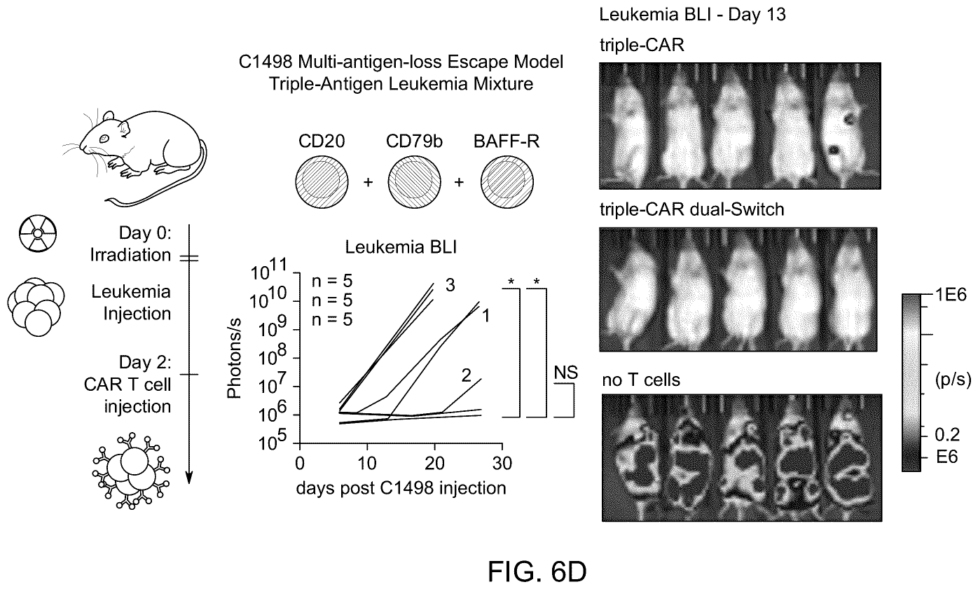

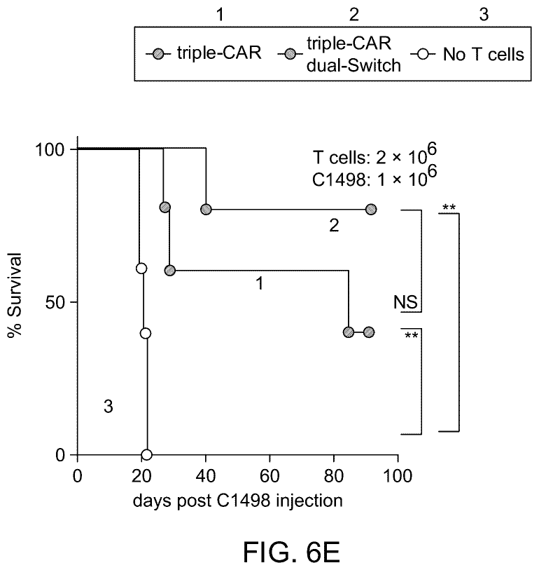

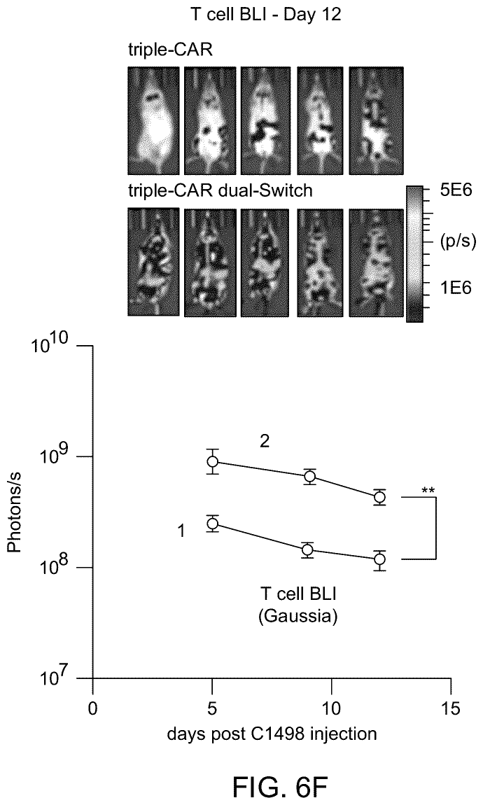

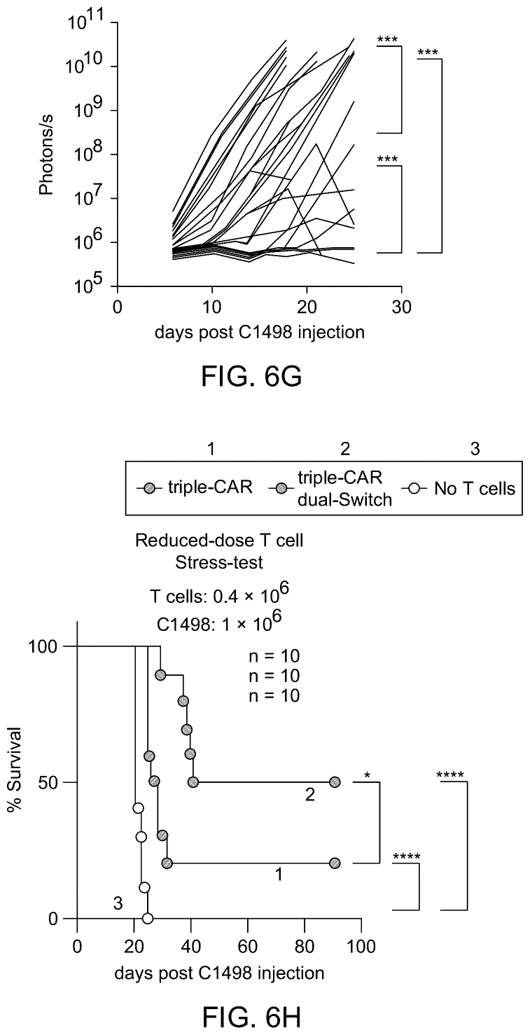

Triple CAR (CD20, CD79b, BAFF-R), or Triple CAR dual switch receptor T cells (FIG. 6A) were zip sorted to obtain highly purified T cells having high surface expression of CARs and switch receptors (FIG. 6B). In vitro target cell viability tests revealed the ability of triple CAR T cells to kill targets expressing either CD20, CD79b, or BAFF-R (FIG. 6C). To test the efficacy of these T cells to eliminate leukemia cancer is vivo, BALB/c mice were injected with a triple antigen C1498 leukemia cell mixture (expressing CD20, CD79b, BAFF-R) followed by administration of triple CAR T cells. Bioluminescence imaging of the animals post injection of the T cells showed that the triple CAR T cells eliminated triple antigen C1498 expressing leukemia cells (FIG. 6D) and enhanced animal survival (FIG. 6E). FIG. 6F shows a greater expansion of triple CAR T cells in vivo as demonstrated by bioluminescence imaging of animals injected with bioluminescent T cells. A comparison of anti-tumor efficacy between Triple CAR and triple CAR dual switch T cells revealed that co-expression of the dual switch receptors improved in vivo anti-tumor activity and survival (FIGS. 6G, 6H).

### 10.2.7 Switch Receptor Arrays Enhance the Anti-Leukemia Activity of Quad-CAR T Cells.

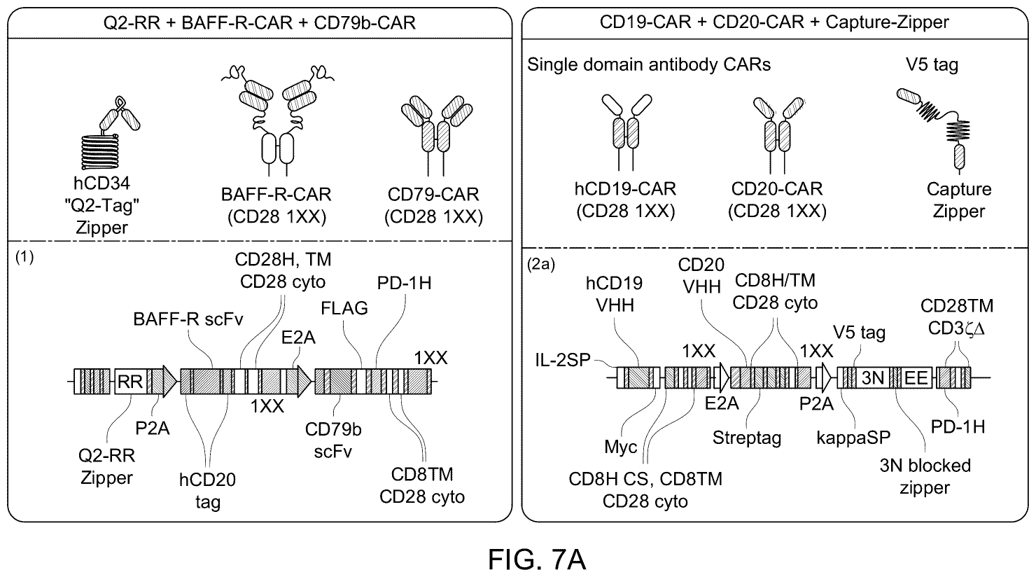

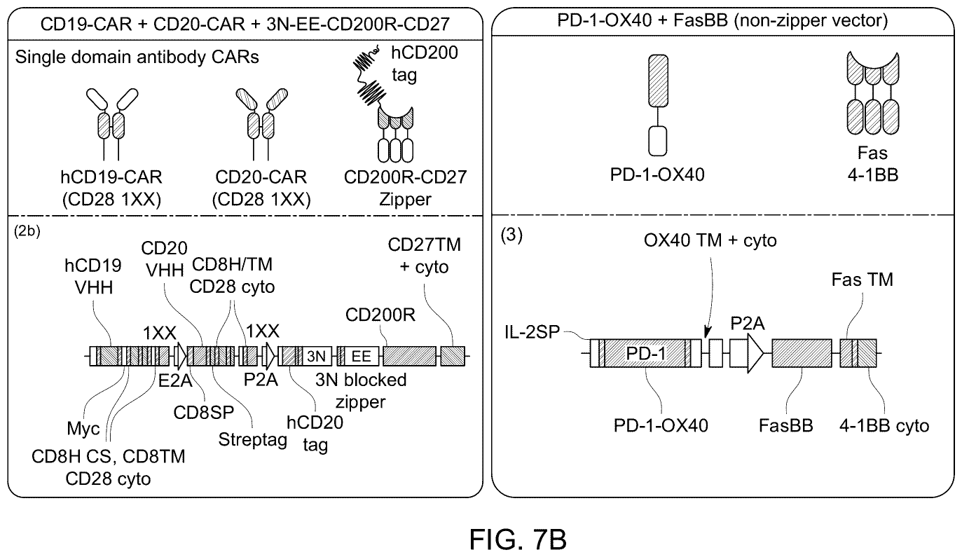

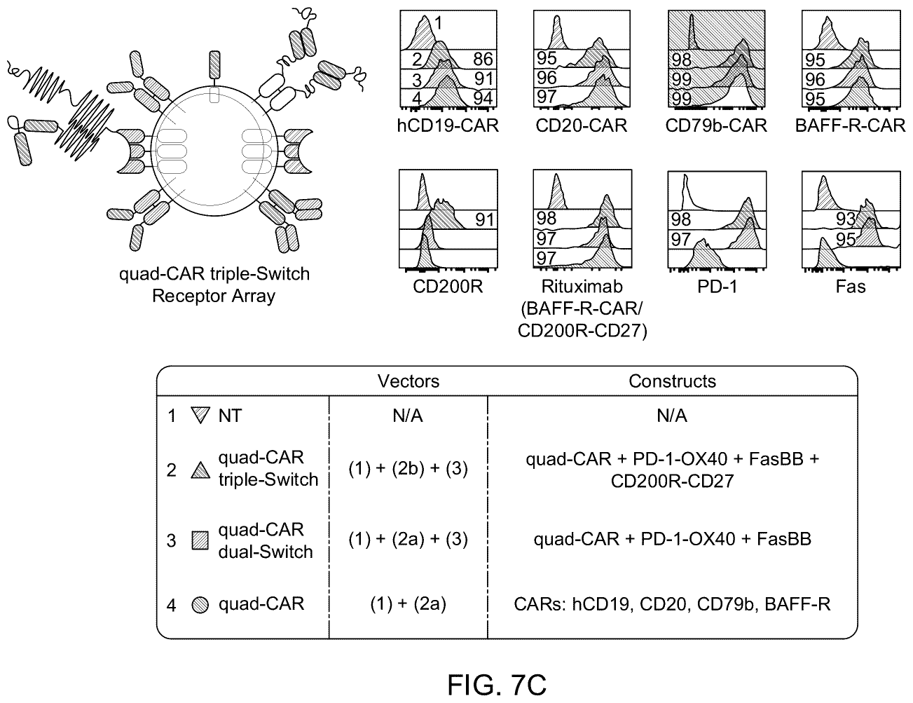

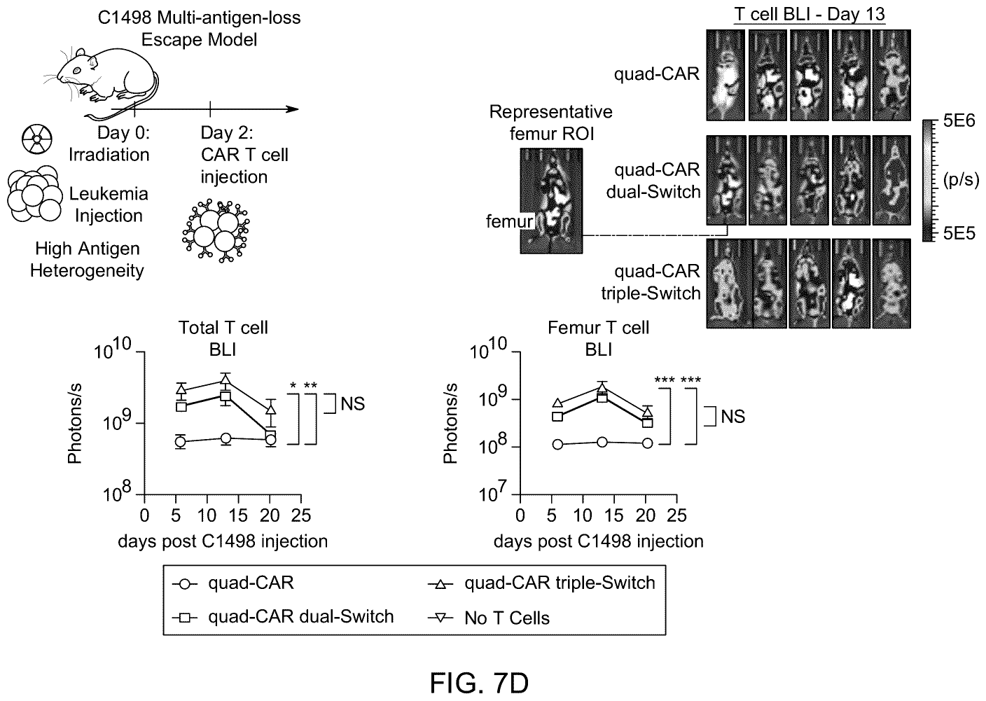

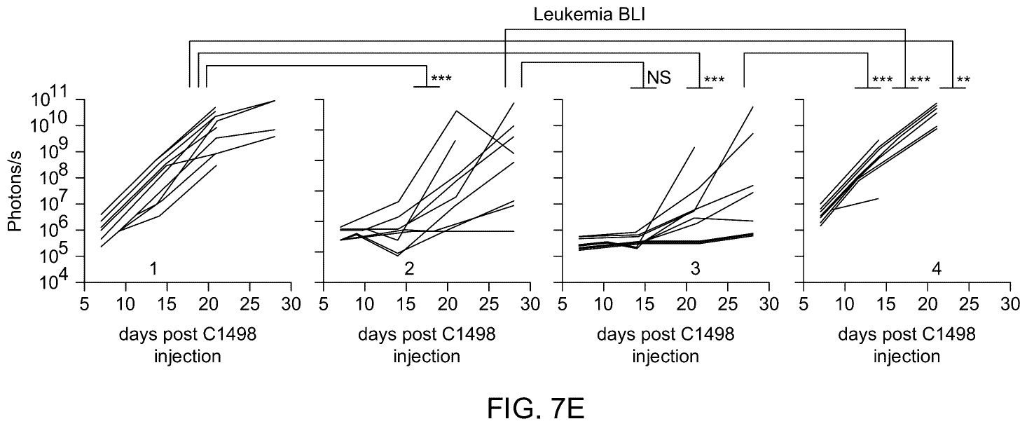

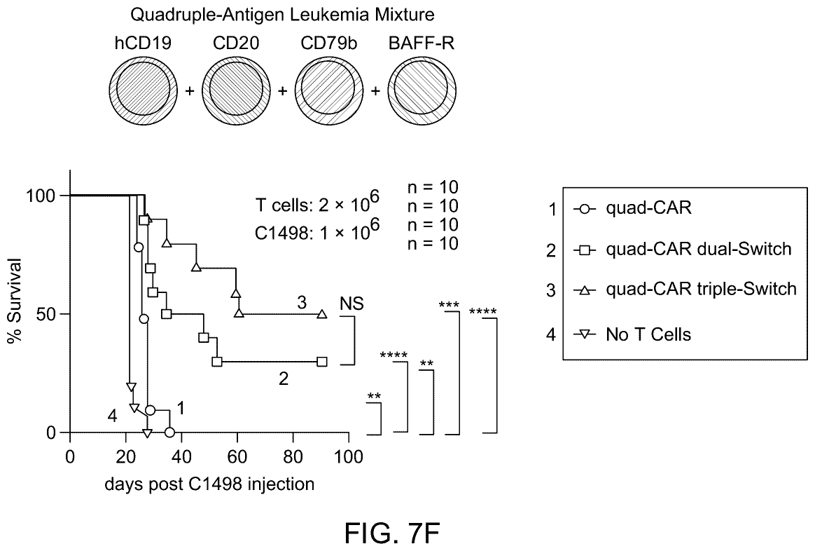

To further test the ability of switch receptors to improve anti-tumor activities of quad-CAR T cells, CAR T cells expressing CD19, CD20, CD79b, and BAFF-R CARs and dual switch (PD-1-OX40, FasBB) or triple switch (PD-1-OX40, FasBB, CD200R1-CD27) receptors were used (FIGS. 7A, 7B). Transduced cells were zip sorted to obtain T cells with high surface expression of the CARs and switch receptors (FIG. 7C). Mice are injected with a mixture of C1498 leukemia expressing CD19, CD20, CD79b, and BAFF-R and treated with Quad-CAR T cells. Bioluminescent imaging of T cells showed enhanced T cell expansion in tumor bearing mice (FIG. 7D). Quad-CAR T cells co-expressing switch receptors showed enhanced anti-tumor activity in a mouse leukemia model using bioluminescent leukemia cells (FIG. 7E). Overall survival of animals was also improved (FIG. 7F).

### 10.2.8 Cognate Switch Receptor: Ligand Interactions Drive Ligand-Specific Expansion of Multi-Switch BCMA-CAR T Cells.

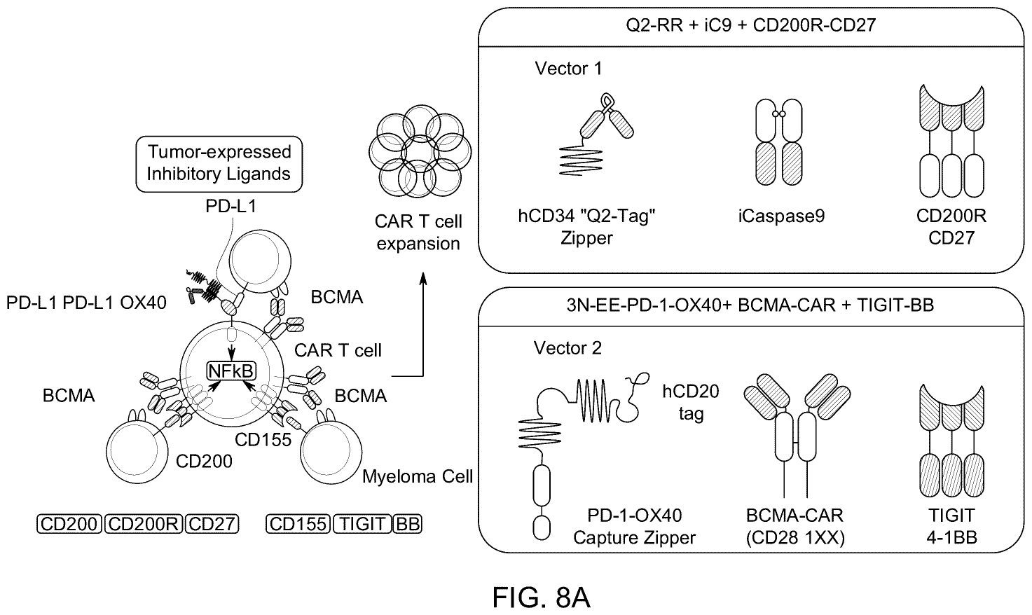

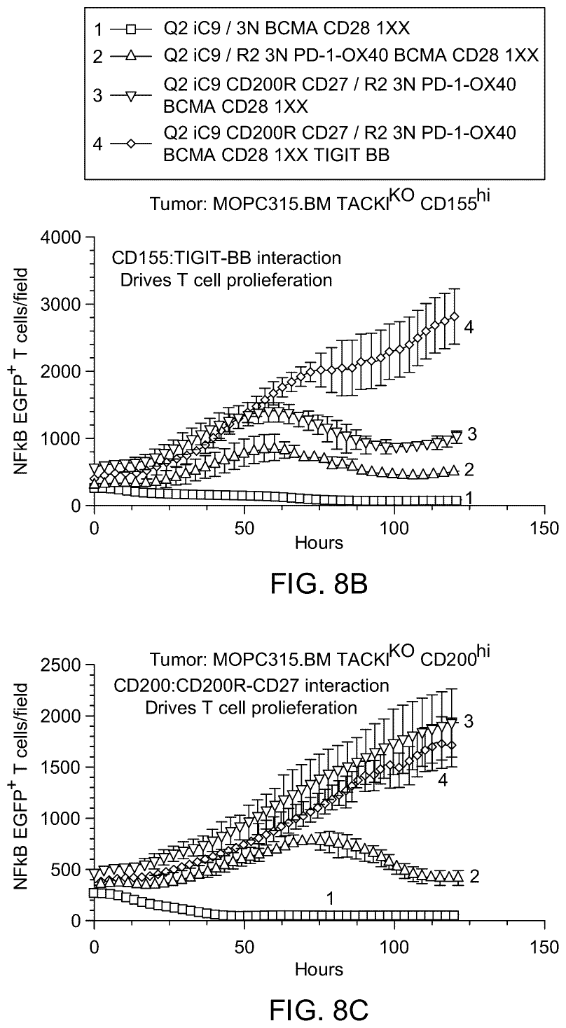

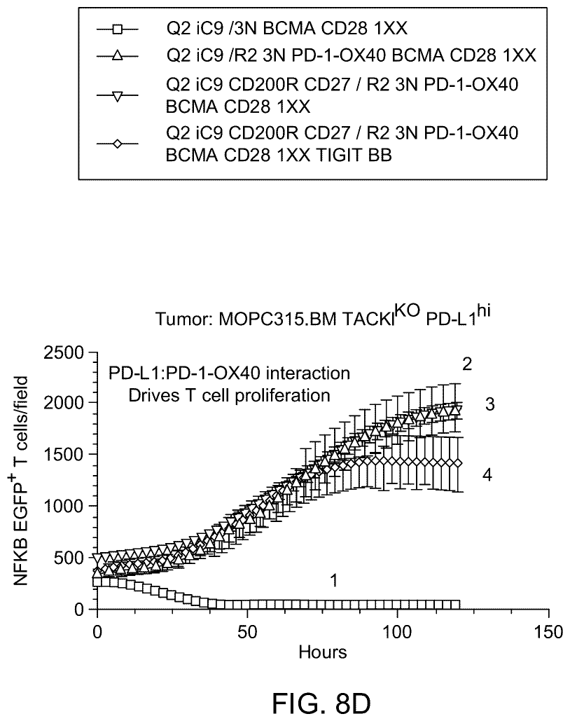

The CAR target BCMA is expressed weakly on the myeloma cell line MOPC315.BM, which impairs expansion of BCMA-CAR T cells, while switch-receptor:ligand interactions strongly promote T cell expansion. Expansion of switch receptor expressing CAR T cells as measured by live cell microscopy imaging of NFkB reporter-expression following encounter with target cells (MOPC315.BM TACI^{KO}) expressing the inhibitory ligands CD155 (FIG. 8B), CD200 (FIG. 8C) or PD-L1 (FIG. 8D). Co-expression of multiple switch receptors was found to enable T cells to proliferate and expand in response to multiple inhibitory ligands simultaneously. This demonstrates that specific switch receptor:inhibitory ligand pair interactions drive CAR T cell proliferation under conditions of insufficient CAR target antigen expression.

### 10.2.9 Dual-Switch Receptor-Expressing BCMA-CAR T Cells Promote Clearance of BCMA Low Multiple Myeloma In Vivo.

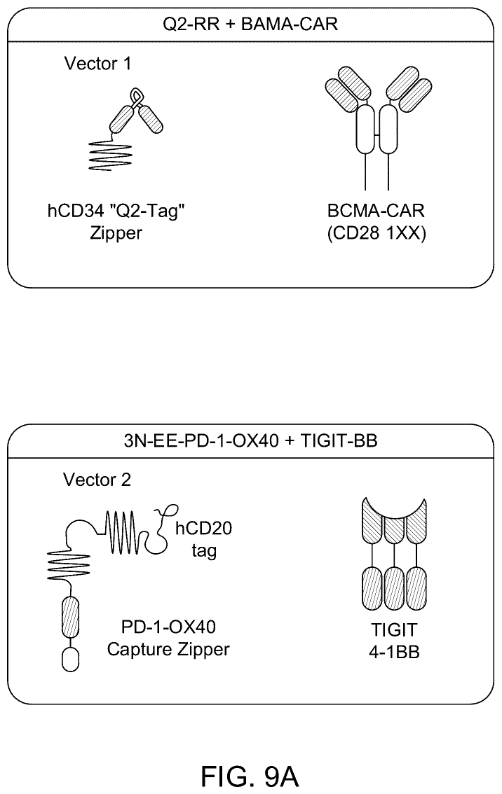

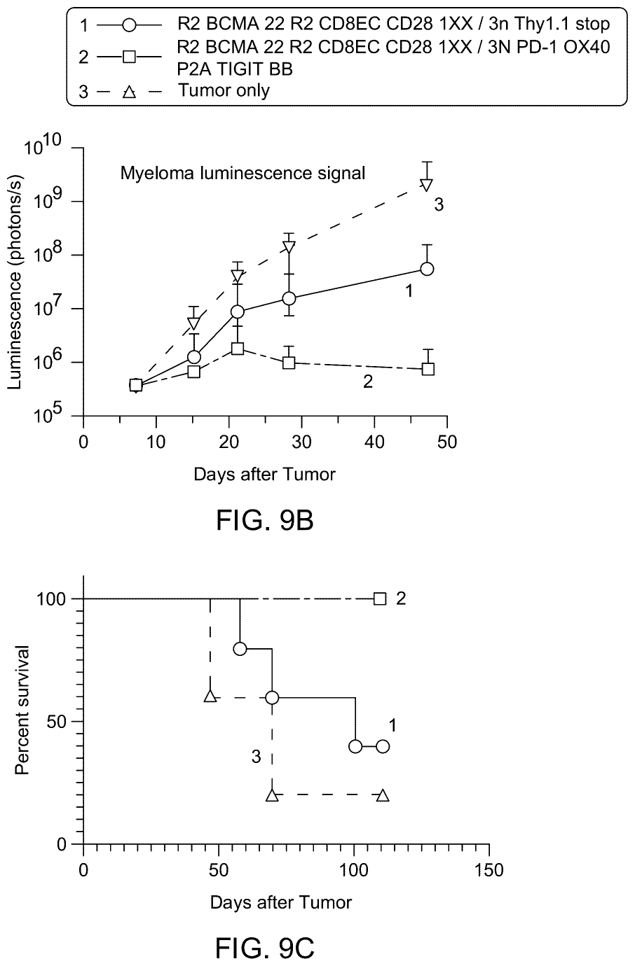

To test the in vivo efficacy of BCMA CAR T cells co-expressing dual switch receptors, BALB/c mice were injected with 4E5 MOPC315.BM TACI^{KO} myeloma tumor cells (BCMA-low model) and treated with a 2E6 dose of BCMA-CAR T cells or BCMA-CAR dual switch receptor (PD-1-OX40, TIGIT-BB) T cells. Bioluminescence imaging (BLI) demonstrated enhanced clearance of the myeloma cells in animals treated with the BCMA CAR dual switch receptor T cells (FIG. 9B). which correlated with increased long-term survival (FIG. 9C).

### 10.2.10 Tim-3, CD70-Targeting Dual-CAR T Cells Co-Expressing Multiple Switch Receptors Demonstrate Increased Antigen-Dependent Proliferation.

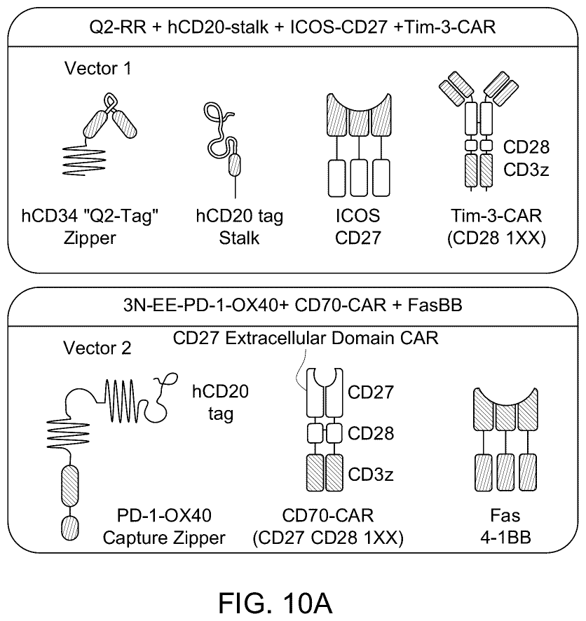

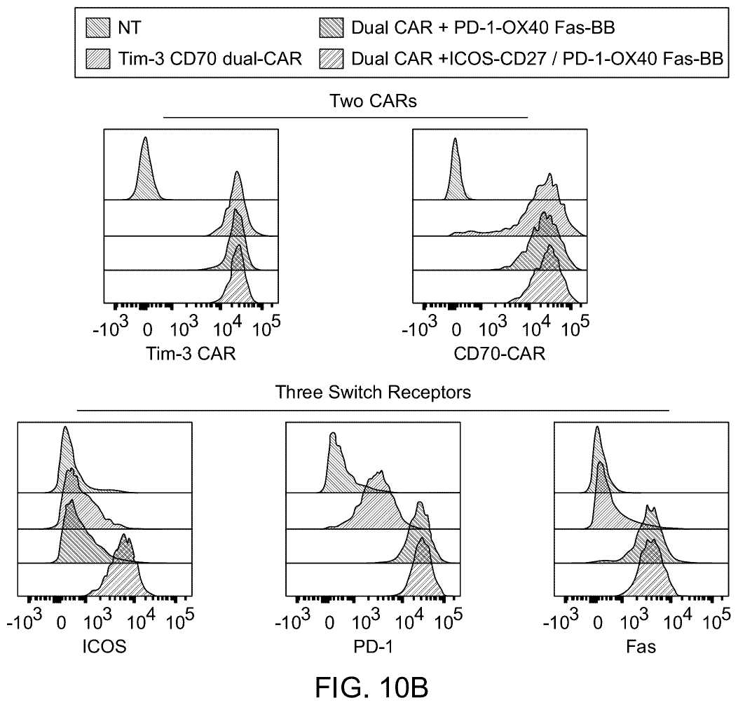

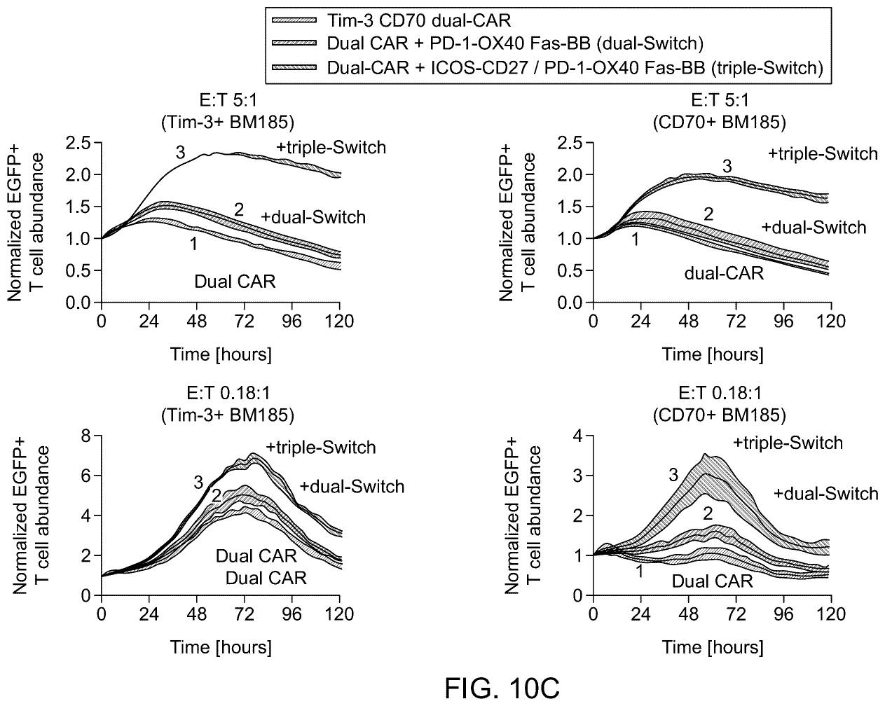

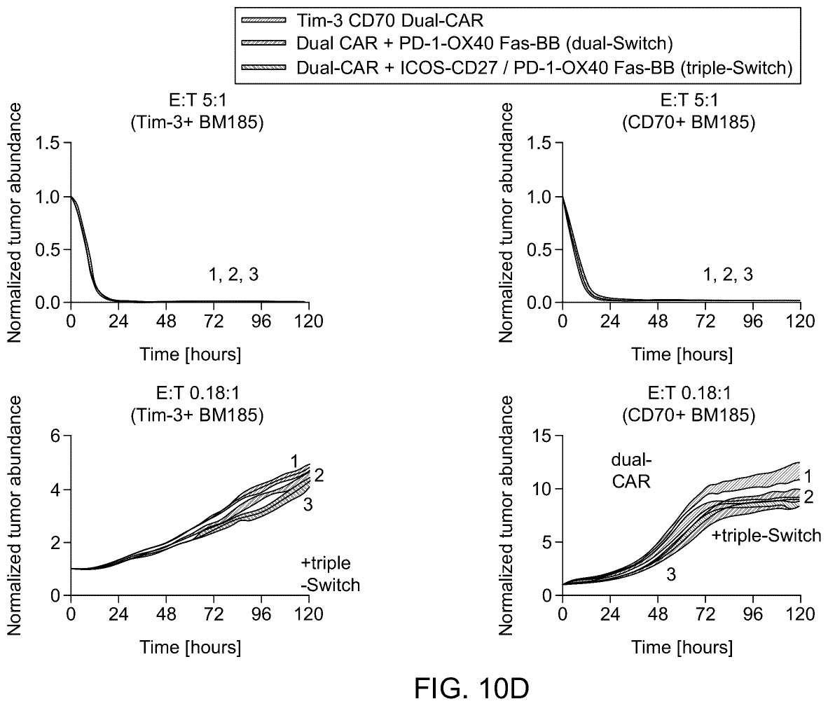

Bulk CD4+CD8 T cells were dual transduced with the vectors depicted in FIG. 10A and zip sorted using anti-CD34 magnetic beads. Flow cytometry demonstrated high purity expression of the Tim-3 CAR (streptavidin tag) and CD70 CAR (V5 tag) CARs and all three switch receptors (FIG. 10B). Tim-3/CD70 dual CAR T cells were co-cultured with Tim-3+ or CD70+BM185 leukemia cells at effector: target ratios (E:T) of 5:1 or 0.18:1. Live-cell imaging analysis (IncuCyte®) was performed to study NFkB reporter-expressing CAR T cells (GFP+) as a measure of T cell expansion. Addition of ICOS-CD27 switch receptor enhanced expansion of dual CAR T cells co-expressing PD-1-OX40 and FasBB switch receptors (FIG. 10C). Tumor abundance studies showed equal efficacy of tumor killing in all dual CAR T cells (FIG. 10D).

### 11.2.11 Co-Expression of Multiple Switch Receptors Enhances Expansion and Anti-Leukemia Activity of CD8+ Tim-3/CD70-Dual CAR T Cells.

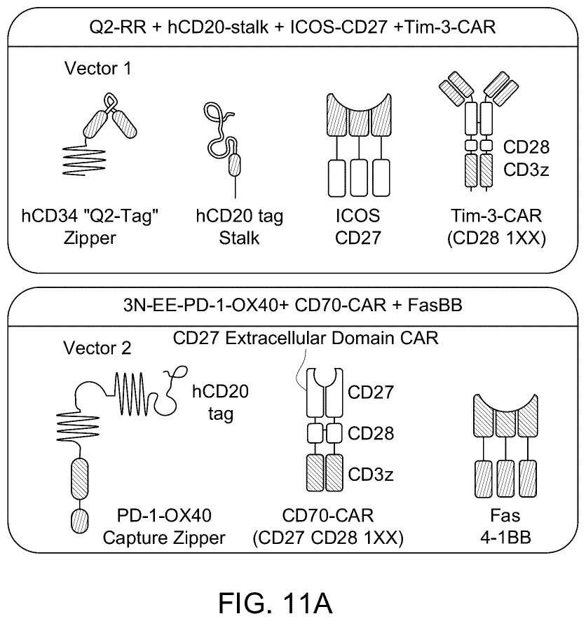

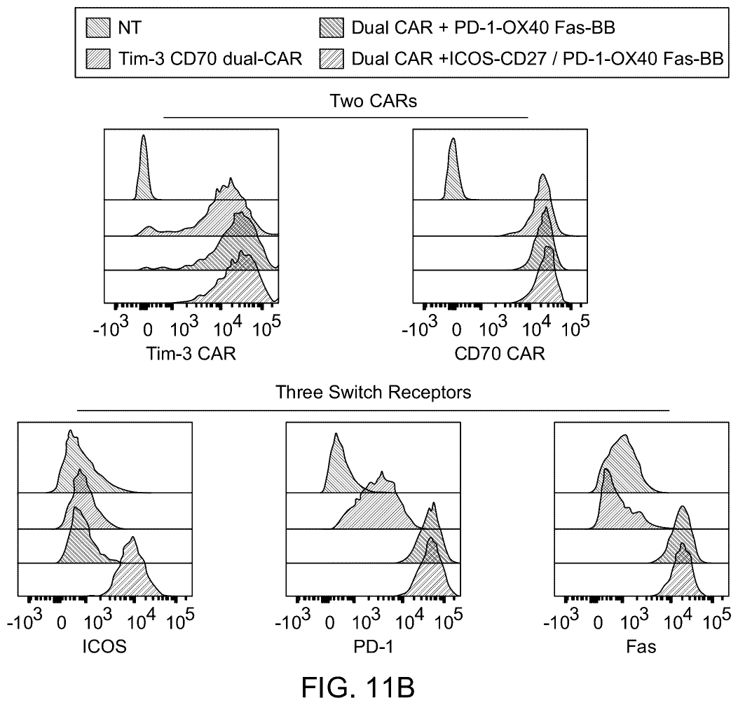

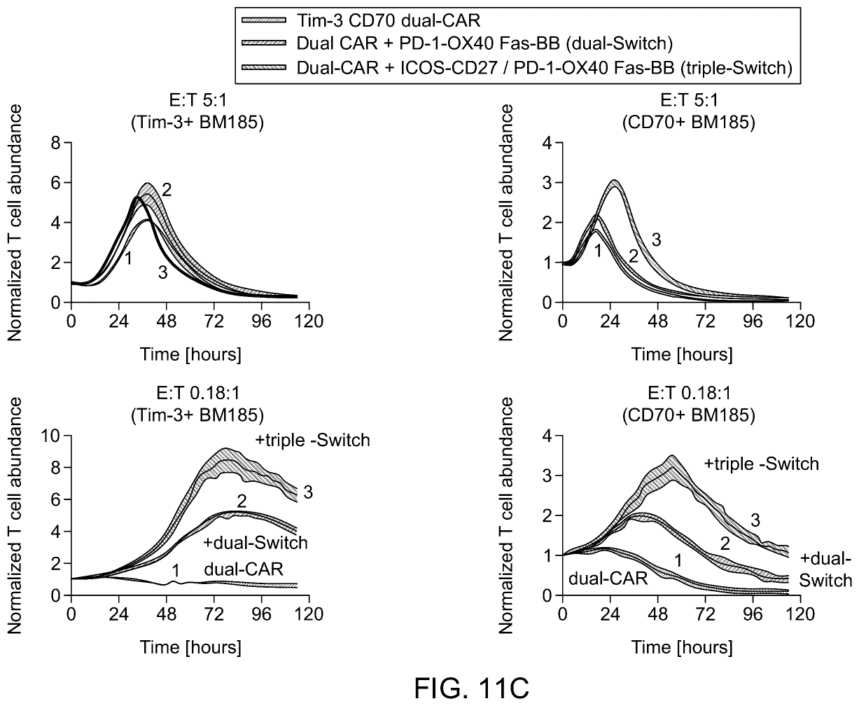

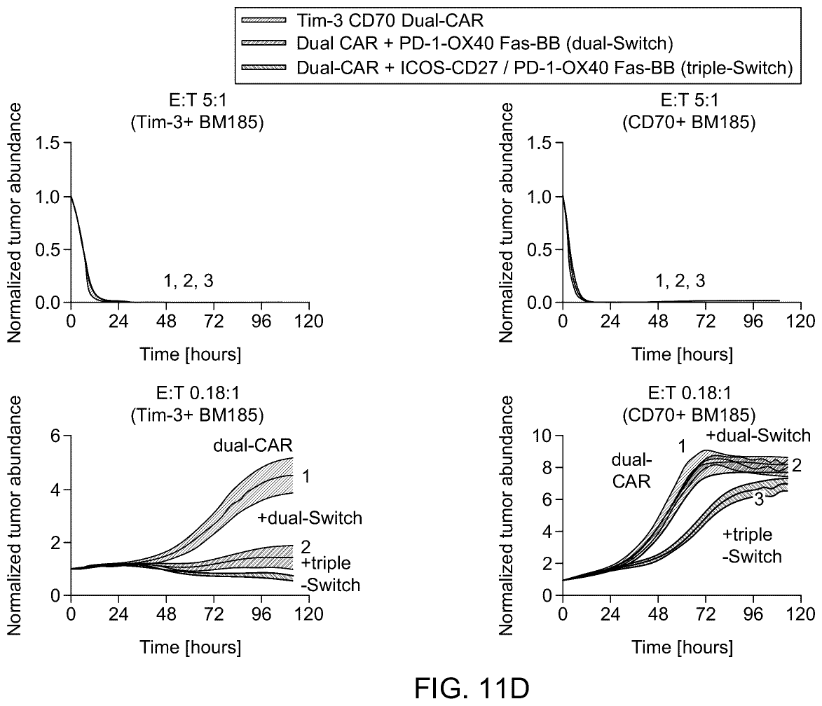

CD8+ T cells were dual transduced with the vectors as depicted in FIG. 11A and zip sorted using anti-CD34 magnetic beads. Flow cytometry demonstrated high purity expression of the Tim-3 CAR (streptavidin tag) and CD70 CAR (V5 tag) CARs and all three switch receptors (FIG. 11B). CD8+ Tim-3/CD70 dual CAR T cells were co-cultured with Tim-3+ or CD70+BM185 leukemia cells at effector: target ratios (E:T) of 5:1 or 0.18:1. Live-cell imaging analysis (IncuCyte®) was performed to study NFkB reporter-expressing CAR T cells (GFP+) as a measure of T cell expansion. Dual CAR T cells proliferated in response to either Tim-3 or CD70⁺ targets, with dual-Switch PD-1-OX40 and FasBB switch receptors enhancing T cell expansion (FIG. 11C). Tumor abundance studies showed enhanced efficacy of iRFP713+ tumor killing at low E:T ratios (bottom row, FIG. 11D).

11.2.12 Co-Expression of Multiple Switch Receptors Enhances Expansion and Anti-Leukemia Activity of CD8+ Tim-3/CD70-Dual CAR T Cells Against Leukemia with Very Low CD70 Expression.

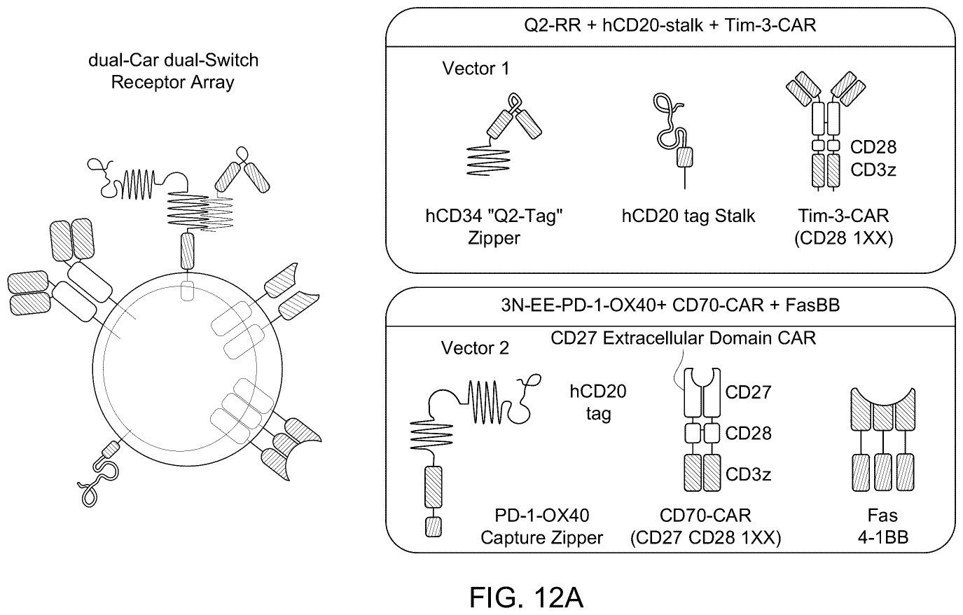

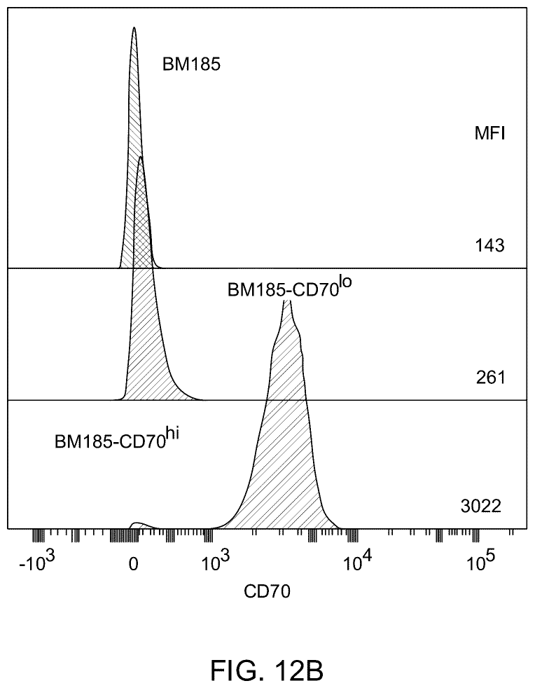

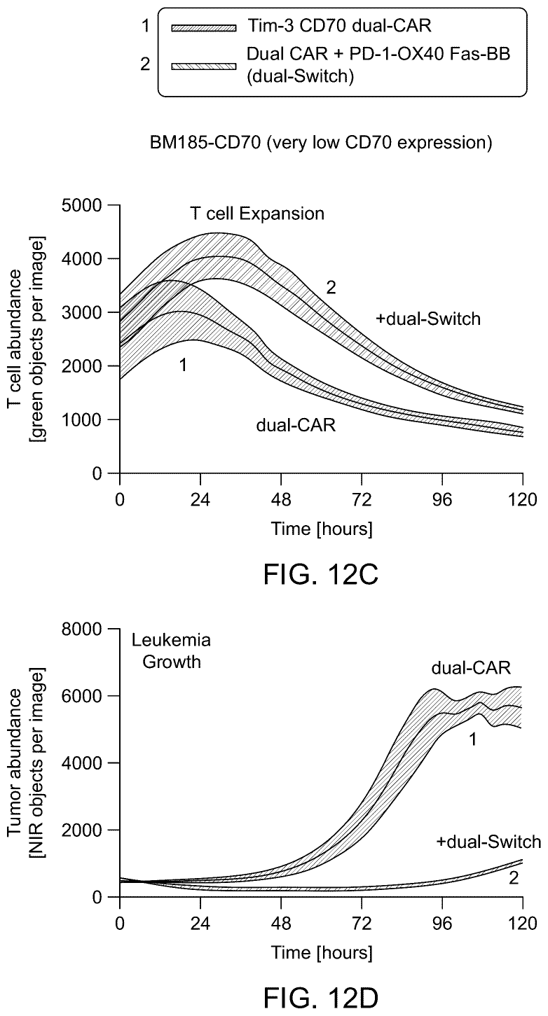

FIG. 12B shows FACS analysis of BM185 cells engineered to express very high CD70 (greater than 20-fold increase in MFI shift) and very low CD70 (less than 2-fold increase in MFI shift) compared with a negative control cell line. Bulk CD4+CD8 T cells were dual transduced with vectors as depicted in FIG. 12A and zip sorted using anti-CD34 magnetic beads. Tim-3/CD70-dual CAR T cells were co-cultured with BM185 leukemia cells with very low CD70 expression (FIG. 12B) at a 5:1 E:T. Live-cell imaging (IncuCyte®) analysis was performed to study expansion of EGFP-tagged CAR T cells and elimination of tumor cells showed that co-expression of PD-1-OX40 and FasBB switch receptors enhanced dual CAR T cell expansion and elimination of antigen-low targets (FIGS. 12C, 12D).

What is claimed is:

1. A system comprising a plurality of nucleic acid constructs, wherein the plurality comprises:
(a) a first nucleic acid construct encoding a membrane bound polypeptide comprising:
(i) an extracellular domain comprising a first leucine zipper sequence;
(ii) a transmembrane domain; and
(iii) an intracellular domain;
(b) a second nucleic acid construct encoding a soluble polypeptide comprising a second leucine zipper sequence capable of heterodimerizing with the first leucine zipper sequence and a signal peptide sequence;
wherein each of the first and second nucleic acid constructs further encode one or more CAR, safety switch, switch receptor, and/or cytokine.

2. The system of claim 1, wherein one or more of the membrane bound polypeptide, the one or more CAR, safety switch, and/or switch receptor comprise an immune checkpoint molecule extracellular, transmembrane, or intracellular domain.

3. The system of claim 2 wherein immune checkpoint molecule is PD-1, Fas, CD200R1, TIGIT, ICOS, CTLA-4, BTLA, TIM-3, LAG-3, LAIR1, HVEM, 2B4 (CD244), CD160, Galectin9, VISTA, or PSGL-1.

4. The system of claim 3, wherein the immune checkpoint molecule:
(a) is PD-1 having an extracellular domain set forth in SEQ ID NO: 52;
(b) is CD200R1 having an extracellular domain set forth in SEQ ID NO: 54; and/or
(c) is Fas having an extracellular domain set forth in SEQ ID NO: 55.

5. The system of any one of claims 1-4, wherein the immune checkpoint molecule is a dominant negative variant thereof.

6. The system of claim 5, wherein the dominant negative receptor selected from PD-1-DNR, Fas-DNR, CD200R1-DNR, TIGIT-DNR, ICOS-DNR, CTLA-4-DNR, BTLA-DNR, TIM-3-DNR, LAG-3-DNR, LAIR1-DNR, HVEM-DNR, CD244-DNR, CD160-DNR, VISTA-DNR, PSGL-1-DNR, variants thereof, or combinations thereof.

7. The system of any one of claims 1-6, wherein the transmembrane domain of one or more of the membrane bound polypeptide, the one or more CAR, safety switch, and/or switch receptor comprises an amino acid sequence set forth in any one of SEQ ID NOs: 56-63.

8. The system of any one of claims 1-6, wherein the intracellular domain of one or more of the membrane bound polypeptide, the one or more CAR, safety switch, and/or switch receptor comprises a CD3 domain, a costimulatory domain, a suicide gene product, survival gene product, fragments thereof, or combinations thereof.

9. The system of claim 8, wherein the suicide gene product is an inducible Caspase 9 polypeptide (iCasp9).

10. The system of claim 9, wherein the iCasp9 has an amino acid sequence set forth in SEQ ID NO: 88.

11. The system of any one of claims 1-10, wherein the survival gene product is a caspase resistant Bcl-2.

12. The system of claim 11, wherein the Bcl-2 has an amino acid sequence set forth in SEQ ID NO: 89.

13. The system of any one of claims 1-11, wherein the intracellular domain of one or more of the membrane bound polypeptide, the one or more CAR, safety switch, and/or switch receptor comprises a member of the TNFR super family.

14. The system of claim 12, wherein the intracellular domain comprises an intracellular domain of OX40, CD40, CD30, 4-1BB, CD27, RANK, GITR, LTBR, HVEM, BAFF-R, TACI, BCMA, TROY, fragments thereof, or combinations thereof.

15. The system of claim 12 or claim 13, wherein the system encodes a switch receptor selected from PD-1-OX40, Fas-4-1BB (FasBB), CD200R1-CD27, TIGIT-4-1BB (TIGITBB), ICOS-CD27, variants thereof, or combinations thereof.

16. The system of any one of claims 1-14, wherein the intracellular domain of one or more of the membrane bound polypeptide, the one or more CAR, safety switch, and/or switch receptor lacks a signaling domain.

17. The system of any one of claims 1-15, wherein the first nucleic acid construct further comprises a rituximab-binding mimotope tag.

18. The system of any one of claims 1-16, wherein the second nucleic acid construct further comprises an enrichment tag selected from the group consisting of CD34 tag (Q2 tag), His tag, Myc-tag, Hemagglutinin (HA)-tag, Flag tag, V5 tag, and T7 tag.

19. The system of any one of claims 1-17, wherein the first nucleic acid construct encodes an amino acid sequence set forth in any one of SEQ ID NOs: 1 or 2.

20. The system of any one of claims 1-18, wherein the first nucleic acid construct encodes a 3N blocked capture zipper.

21. The system of any one of claims 1-20, wherein the first nucleic acid construct encodes an amino acid sequence set forth in any one of SEQ ID NOs: 3 or 4.

22. The system of any one of claims 1-21, wherein the extracellular domain of one or more of the membrane bound polypeptide, the one or more CAR, safety switch, and/or switch receptor further comprises:
a) a linker; and/or
b) a spacer/hinge domain between the extracellular domain and the transmembrane domain.

23. The system of claim 22, wherein:
the linker has an amino acid sequence set forth in any one of SEQ ID NOs: 17-41;
the spacer/hinge domain has an amino acid sequence set forth in any one of SEQ ID NOs: 42, 43-51, or 53.

24. The system of any one of claims 1-23, wherein the transmembrane domain has an amino acid sequence set forth in any one of SEQ ID NOs: 56-63.

25. The system of any one of claims 1-24, wherein each CAR comprises an antigen binding domain, a transmembrane domain, and an intracellular signaling domain.

26. The system of claim 25, wherein each CAR binds to a cell surface antigen selected from, CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, BAFF-R, CD79, PD-1, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, ZDHHC11, variants thereof, or combinations thereof.

27. The system of claim 25, wherein each CAR binds to a cell surface antigen selected from BAFF-R, CD79, CD19, CD20, CD70, PD-1, Tim-3, or variants thereof.

28. The system of any one of claims 1-27, wherein the system comprises one or more nucleic acid constructs encoding a sequence set forth in any one of SEQ ID NO: 105-122.

29. An engineered immune cell comprising the system of any one of claims 1-28.

30. The engineered immune cell of claim 29, wherein the immune cell is a T cell.

31. A method of modifying a cell comprising delivering to the cell, the system of any one of claims 1-28.

32. The method of claim 31, wherein the cell is a mammalian cell.

33. The method of claim 32, wherein the mammalian cell is an immune cell.

34. The method of claim 33, wherein the immune cell is a T cell.

35. A method for enriching a population of modified cells comprising:
(a) delivering to the cell, the system of any one of claims 1-28 to obtain a population of cells comprising modified cells;
(b) culturing the population of cells; and
(c) enriching for the population of modified cells by selecting for expression of the enrichment tag.

36. A method for improving T cell function comprising delivering to the T cell, the system of any one of claims 1-28 to obtain a modified T cell, wherein the modified T cell exhibits at least one characteristic selected from enhanced proliferation, enhanced survival, enhanced persistence, enhanced activation, and reduced exhaustion compared to a control, unmodified T cell.

37. The method of claim 36, wherein the modified population of T cells exhibits attenuated signaling in response to PD-1, Fas, CD200R1, TIGIT, ICOS, CTLA-4, BTLA, TIM-3, VISTA, PSGL-1, LAG-3, LAIR1, HVEM, 2B4 (CD244), CD160, and/or Galectin9 specific ligands, compared to the population of T cells before the delivering step.

38. The method of claim 36, wherein the modified population of T cells exhibits enhanced OX40, CD40, CD30, 4-1BB, CD27, RANK, GITR, LTBR, HVEM, BAFF-R, TACI, BCMA, and/or TROY signaling in response to binding of one or more of PD-1, Fas, CD200R1, TIGIT, ICOS, CTLA-4, BTLA, TIM-3, VISTA, PSGL-1, LAG-3, LAIR1, HVEM, 2B4 (CD244), CD160, and/or Galectin9 specific ligands, compared to the population of T cells before the delivering step.

39. A method of treating a disease comprising administering to a subject in need thereof:
(a) a population of modified cells comprising the system of any one of claims 1-28;
(b) a cell modified according to the method of any one of claims 31-34; and/or
(c) an enriched population of cells according to the method of claim 35.

40. The method of claim 39, further comprising, culturing the T cells with a small molecular weight inhibitor before administering to the subject.

41. The method of claim 40, wherein the small molecular weight inhibitor is a Src kinase inhibitor.

42. The method of claim 41, wherein the Src inhibitor is Dasatinib.

43. The method of any one of claims 39-42, wherein the subject is a human subject.

44. The method of any one of claims 39-42, wherein the disease is a cancer, an autoimmune disease, an inflammatory disease, or a graft versus-host disease.

45. The method of claim 44, wherein the cancer is leukemia, lymphoma, myeloma, ovarian cancer, breast cancer, bladder cancer, brain cancer, colon cancer, intestinal cancer, liver cancer, lung cancer, pancreatic cancer, prostate cancer, testicular cancer, anal cancer, skin cancer, stomach cancer, glioblastoma, throat cancer, melanoma, neuroblastoma, adenocarcinoma, glioma, or soft tissue sarcoma.

46. The method of claim 45, wherein the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia.

47. The method of claim 45, wherein the lymphoma is Hodgkin's lymphoma or non-Hodgkin's lymphoma.

48. The method of claim 47, where the non-Hodgkin's lymphoma is B-cell non-Hodgkin's lymphoma or T-cell non-Hodgkin's lymphoma.

49. The method of any one of claims 39-48, wherein the cancer comprises cells expressing BAFF-R, CD79, CD70, CD19, CD20, PD-1, Tim-3, variants thereof or combinations thereof.

50. The method of any one of claims 39-49, wherein the cancer comprises cells expressing at least one antigen selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, ZDHHC11 variants thereof, or combinations thereof.
